(19) United States
(12) Patent Application Publication
(10) Pub. No.: US 2010/0160305 A1
(43) Pub. Date: Jun. 24, 2010

(54) 3, 4, 5 - Substituted Piperidine Compounds

(75) Inventors: Keiichi Masuya, Bottmingen (CH); Fumiaki Yokokawa, Tsukuba (JP); Osamu Irie, Tsukuba (JP); Atsuko Nihonyanagi, Tsukuba (JP); Takeru Ehara, Tsukuba (JP); Kazuhide Konishi, Tsukuba (JP); Takanori Kanazawa, Tsukuba (JP); Masaki Suzuki, Tsukuba (JP)

Correspondence Address: NOVARTIS INSTITUTES FOR BIOMEDICAL RESEARCH, INC., 220 MASSACHUSETTS AVENUE, CAMBRIDGE, MA, 02139, US

(73) Assignee: NOVARTIS AG, Basel (CH)

(21) Appl. No.: 11/908,182

(22) PCT Filed: Mar. 7, 2006
(86) PCT No.: PCT/EP2006/002083

§ 371 Date: Mar. 4, 2010

(30) Foreign Application Priority Data
Mar. 9, 2005 (GB) 0504850.9

(51) Int. Cl. A61K 31/451; A61K 31/538; A61K 31/454; C07D 413/12; C07D 401/12; C07D 211/60; A61P 43/00

(52) U.S. Cl. 514/230.5; 514/323; 514/327; 514/329; 514/330; 544/105; 546/201; 546/221; 546/224; 546/225

(57) ABSTRACT

3,4,5-substituted piperidine compounds, these compounds for use in the diagnostic and therapeutic treatment of a warm-blooded animal, especially for the treatment of a disease (=disorder) that depends on activity of renin; the use of a compound of that class for the preparation of a pharmaceutical formulation for the treatment of a disease that depends on activity of renin; the use of a compound of that class in the treatment of a disease that depends on activity of renin; pharmaceutical formulations comprising a 3,4,5-substituted piperidine compound, and/or a method of treatment comprising administering a 3,4,5-substituted piperidine compound, a method for the manufacture of a 3,4,5-substituted piperidine compound, and novel intermediates and partial steps for its synthesis.

The compounds are of the formula I,

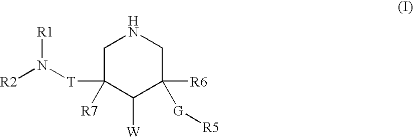

wherein the substitutents are as defined in the specification.

The invention relates to 3,4,5-substituted piperidine compounds, these compounds for use in the diagnostic and therapeutic treatment of a warm-blooded animal, especially for the treatment of a disease (=disorder) that depends on activity of renin; the use of a compound of that class for the preparation of a pharmaceutical formulation for the treatment of a disease that depends on activity of renin; the use of a compound of that class in the treatment of a disease that depends on activity of renin; pharmaceutical formulations comprising a 3,4,5-substituted piperidine compound, and/or a method of treatment comprising administering a 3,4,5-substituted piperidine compound, a method for the manufacture of a 3,4,5-substituted piperidine compound, and novel intermediates and partial steps for its synthesis.

The present invention relates to a compound of the formula I

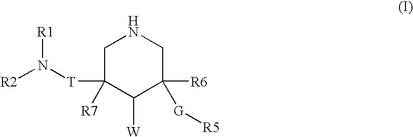

wherein

R1 is hydrogen, unsubstituted or substituted alkyl, unsubstituted or substituted alkenyl, unsubstituted or substituted alkynyl, unsubstituted or substituted aryl, unsubstituted or substituted heterocyclyl or unsubstituted or substituted cycloalkyl;

R2 isunsubstituted or substituted alkyl, unsubstituted or substituted alkenyl, unsubstituted or substituted alkynyl, unsubstituted or substituted aryl, unsubstituted or substituted heterocyclyl, unsubstituted or substituted cycloalkyl, or acyl;

W is a moiety selected from those of the formulae IA, IB and IC,

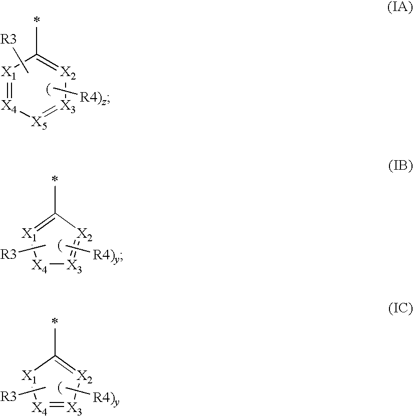

wherein the asterisk (*) denotes the position where the moiety W is bound to the 4-carbon in the piperidine ring in formula I, and wherein

X₁, X₂, X₃, X₄ and X₅ are independently selected from carbon and nitrogen, where X₄ in formula IB and X₁ in formula IC may have one of these meanings or alternatively be selected from S and O, where carbon and nitrogen ring atoms can carry the required number of hydrogen or substituents R₃ or (if present within the limitations given below) R₄ to complete the number of bonds emerging from a ring carbon to four, from a ring nitrogen to three; with the proviso that in formula IA at least 2, preferably at least 3 of X₁ to X₅ are carbon and in formulae IB and IC at least one of X₁ to X₄ is carbon, preferably at least two of X₁ to X₄ are carbon;

y is 0, 1, 2 or 3;

z is 0, 1, 2, 3 or 4

(the obligatory moiety) R3 which can only be bound to any one of X₁, X₂, X₃ and X₄ (instead of a hydrogen and replacing it) is unsubstituted or substituted C₁-C₇-alkyl, unsubstituted or substituted C₂-C₇-alkenyl, unsubstituted or substituted C₂-C₇-alkynyl, unsubstituted or substituted aryl, unsubstituted or substituted heterocyclyl, unsubstituted or substituted cycloalkyl, halo, hydroxy, etherified or esterified hydroxy, unsubstituted or substituted mercapto, unsubstituted or substituted sulfinyl (—S(═O)—), unsubstituted or substituted sulfonyl (—S(═O)₂—), amino, mono- or di-substituted amino, carboxy, esterified or amidated carboxy, unsubstituted or substituted sulfamoyl, nitro or cyano, with the proviso that if R3 is hydrogen then y and z are 0 (zero);

R4 (which is preferably bound to a ring atom other than that to which R₃ is bound) is—if y or z is 2 or more, independently—selected from a group of substituents consisting of unsubstituted or substituted C₁-C₇-alkyl, unsubstituted or substituted C₂-C₇-alkenyl, unsubstituted or substituted C₂-C₇-alkynyl, halo, hydroxy, etherified or esterified hydroxy, unsubstituted or substituted mercapto, unsubstituted or substituted sulfinyl (—S(═O)—), unsubstituted or substituted sulfonyl (—S(═O)₂—), amino, mono- or di-substituted amino, carboxy, esterified or amidated carboxy, unsubstituted or substituted sulfamoyl, nitro and cyano;

G is methylene, oxy (—O—), thio (—S—), imino (—NH—) or substituted imino (—NR8-) wherein R8 is unsubstituted or substituted alkyl; and

R5 is hydrogen, unsubstituted or substituted alkyl, unsubstituted or substituted aryl or acyl or, if G is methylene, can have one of these meanings or alternatively be unsubstituted or substituted alkyloxy;

or G-R5 is halo;

R6 is hydrogen, C₁-C₇-alkyl or halo;

or G-R5 and R6 together are oxo (═O) and/or G-R5 is hydroxy and R6 is hydroxy;

R7 is hydrogen, hydroxy, halo, C₁-C₇-alkyl, cycloalkyl, halo-substituted cycloalkyl, C₁-C₇-alkoxy, halo-C₁-C₇-alkoxy or cyano; and

T is carbonyl (—C(═O)—);

or a (preferably pharmaceutically acceptable) salt thereof.

The compounds of the present invention exhibit inhibitory activity on the natural enzyme renin. Thus, compounds of formula I may be employed for the treatment (this term also including prophylaxis) of one or more disorders or diseases selected from, inter alia, hypertension, atherosclerosis, unstable coronary syndrome, congestive heart failure, cardiac hypertrophy, cardiac fibrosis, cardiomyopathy postinfarction, unstable coronary syndrome, diastolic dysfunction, chronic kidney disease, hepatic fibrosis, complications resulting from diabetes, such as nephropathy, vasculopathy and neuropathy, diseases of the coronary vessels, restenosis following angioplasty, raised intra-ocular pressure, glaucoma, abnormal vascular growth and/or hyperaldosteronism, and/or further cognitive impairment, alzheimers, dementia, anxiety states and cognitive disorders, especially as far as these diseases can be modulated by renin inhibition.

Listed below are definitions of various terms used to describe the compounds of the present invention as well as their use and synthesis, starting materials and intermediates and the like. These definitions, either by replacing one, more than one or all general expressions or symbols used in the present disclosure and thus yielding preferred embodiments of the invention, preferably apply to the terms as they are used throughout the specification unless they are otherwise limited in specific instances either individually or as part of a larger group.

The term “lower” or “C₁-C₇-” defines a moiety with up to and including maximally 7, especially up to and including maximally 4, carbon atoms, said moiety being branched (one or more times) or straight-chained and bound via a terminal or a non-terminal carbon. Lower or C₁-C₇-alkyl, for example, is n-pentyl, n-hexyl or n-heptyl or preferably C₁-C₄-alkyl, especially as methyl, ethyl, n-propyl, sec-propyl, n-butyl, isobutyl, sec-butyl, tert-butyl.

Halo or halogen is preferably fluoro, chloro, bromo or iodo, most preferably fluoro, chloro or bromo; where halo is mentioned, this can mean that one or more (e.g. up to three) halogen atoms are present, e.g. in halo-C₁-C₇-alkyl, such as trifluoromethyl, 2,2-difluoroethyl or 2,2,2-trifluoroethyl.

Unsubstituted or substituted alkyl is preferably C₁-C₂₀-alkyl, more preferably C₁-C₇-alkyl, that is straight-chained or branched (one or, if desired and possible, more times), and which is unsubstituted or substituted by one or more, e.g. up to three moieties selected from unsubstituted or substituted aryl as described below, especially phenyl or naphthyl each of which is unsubstituted or substituted as described below for unsubstituted or substituted aryl, unsubstituted or substituted heterocyclyl as described below, especially pyrrolyl, furanyl, thienyl (=thiophenyl), thiazolyl, pyrazolyl, triazolyl, tetrazolyl, oxetidinyl, 3-(C₁-C₇-alkyl)-oxetidinyl, pyridyl, pyrimidinyl, morpholino, thiomorpholino, piperidinyl, piperazinyl, pyrrolidinyl, tetrahydrofuran-onyl, tetrahydro-pyranyl, indolyl, 1H-indazanyl, benzofuranyl, benzothiophenyl, quinolinyl, isoquinolinyl, 1,2,3,4-tetrahydro-1,4-benzoxazinyl, 2H-1,4-benzoxazin-3(4H)-onyl, 2H,3H-1,4-benzodioxinyl or benzo[1,2,5]oxadiazolyl each of which is unsubstituted or substituted as described below for unsubstituted or substituted heterocyclyl, in the case of R5 especially by one or more, e.g. one to three substitutents independently selected from halo, such as chloro, C₁-C₇-alkyl, such as methyl, and C₁-C₇-alkanoyl, such as acetyl; unsubstituted or substituted cycloalkyl as described below, especially cyclopropyl, cyclobutyl, cyclopentyl or cyclohexyl each of which is unsubstituted or substituted as described below for unsubstituted or substituted cycloalkyl, halo, hydroxy, C₁-C₇-alkoxy, halo-C₁-C₇-alkoxy, such as trifluoromethoxy, hydroxy-C₁-C₇-alkoxy, C₁-C₇-alkoxy-C₁-C₇-alkoxy, phenyl- or naphthyloxy, phenyl- or naphthyl-C₁-C₇-alkyloxy, C₁-C₇-alkanoyloxy, benzoyl- or naphthoyloxy, C₁-C₇-alkylthio, halo-C₁-C₇-alkylthio, such as trifluoromethylthio, C₁-C₇-alkoxy-C₁-C₇-alkylthio, phenyl- or naphthylthio, phenyl- or naphthyl-C₁-C₇-alkylthio, C₁-C₇-alkanoylthio, benzoyl- or naphthoylthio, nitro, amino, mono- or di-(C₁-C₇-alkyl and/or C₁-C₇-alkoxy-C₁-C₇alkyl)-amino, mono- or di-(naphthyl- or phenyl-C₁-C₇-alkyl)-amino, C₁-C₇-alkanoylamino, benzoyl- or naphthoylamino, C₁-C₇-alkylsulfonylamino, phenyl- or naphthylsulfonylamino wherein phenyl or naphthyl is unsubstituted or substituted by one or more, especially one to three, C₁-C₇-alkyl moieties, phenyl- or naphthyl-C₁-C₇-alkylsulfonylamino, carboxyl, C₁-C₇-alkyl-carbonyl, C₁-C₇-alkoxy-carbonyl, phenyl- or naphthyloxycarbonyl, phenyl- or naphthyl-C₁-C₇-alkoxycarbonyl, carbamoyl, N-mono- or N,N-di-(C₁-C₇-alkyl)-aminocarbonyl, N-mono- or N,N-di-(naphthyl- or phenyl-C₁-C₇-alkyl)-aminocarbonyl, cyano, C₁-C₇-alkenylene or -alkynylene, C₁-C₇-alkylenedioxy, sulfenyl (—S—OH), sulfinyl (—S(═O)—OH), C₁-C₇-alkylsulfinyl (C₁-C₇-alkyl-S(═O)—), phenyl- or naphthylsulfinyl wherein phenyl or naphthyl is unsubstituted or substituted by one or more, especially one to three, C₁-C₇-alkyl moieties, phenyl- or naphthyl-C₁-C₇-alkylsulfinyl, sulfonyl (—S(O)₂OH), C₁-C₇-alkylsulfonyl (C₁-C₇-alkyl-SO₂—), phenyl- or naphthylsulfonyl wherein phenyl or naphthyl is unsubstituted or substituted by one or more, especially one to three, C₁-C₇-alkyl moieties, phenyl- or naphthyl-C₁-C₇-alkylsulfonyl, sulfamoyl, N-mono or N,N-di-(C₁-C₇-alkyl, phenyl, naphthyl, phenyl-C₁-C₇-alkyl or naphthyl-C₁-C₇-alkyl)-aminosulfonyl and in position 2 or higher with regard to the binding carbon (as otherwise unsubstituted or substituted alkanoyl would result which falls under acyl) oxo.

Unsubstituted or substituted alkenyl preferably has 2 to 20 carbon atoms and includes one or more double bonds, and is more preferably C₂-C₇-alkenyl that is unsubstituted or substituted as described above for unsubstituted or substituted alkyl. Examples are vinyl or allyl.

Unsubstituted or substituted alkynyl preferably has 2 to 20 carbon atoms and includes one or more triple bonds, and is more preferably C₂-C₇-alkynyl that is unsubstituted or substituted as described above for unsubstituted or substituted alkyl. An example is prop-2-ynyl.

Unsubstituted or substituted aryl preferably is a mono- or bicyclic aryl moiety with 6 to 22 carbon atoms, especially phenyl (very preferred), or naphthyl (very preferred), and is unsubstituted or substituted by one or more, especially one to three, moieties, preferably independently selected from the group consisting of

a substituent of the formula —(C₀-C₇-alkylene)-(X)ᵣ-(C₁-C₇-alkylene)-(Y)ₛ—(C₀-C₇-alkylene)-H where C₀-alkylene means that a bond is present instead of bound alkylene, r and s, each independently of the other, are 0 or 1 and each of X and Y, if present and independently of the others, is —O—, —NV—, —S—, —C(═O)—, —C(═S), —O—CO—, —CO—O—, —NV—CO—; —CO—NV—; —NV—SO₂—, —SO₂—NV; —NV—CO—NV—, —NV—CO—O—, —O—CO—NV—, —NV—SO₂—NV— wherein V is hydrogen or unsubstituted or substituted alkyl as defined below; especially selected from C₁-C₇-alkyl, phenyl, naphthyl, phenyl- or naphthyl-C₁-C₇-alkyl and halo-C₁-C₇-alkyl; e.g. C₁-C₇-alkyl, such as methyl, ethyl, n-propyl, isopropyl, n-butyl, isobutyl, sec-butyl or tert-butyl, hydroxy-C₁-C₇-alkyl, C₁-C₇-alkoxy-C₁-C₇-alkyl, such as 3-methoxypropyl or 2-methoxyethyl, C₁-C₇-alkoxy-C₁-C₇-alkoxy-C₁-C₇-alkyl, C₁-C₇-alkanoyloxy-C₁-C₇-alkyl, C₁-C₇-alkyloxycarbonyl-C₁-C₇-alkyl, amino-C₁-C₇-alkyl, such as aminomethyl, (N—) mono- or (N,N—) di-(C₁-C₇-alkyl)-amino-C₁-C₇-alkyl, C₁-C₇-alkoxy-C₁-C₇-alkylamino-C₁-C₇-alkyl, mono-(naphthyl- or phenyl)-amino-C₁-C₇-alkyl, mono-(naphthyl- or phenyl-C₁-C₇-alkyl)-amino-C₁-C₇-alkyl, C₁-C₇-alkanoylamino-C₁-C₇-alkyl, C₁-C₇-alkyl-O—CO—NH—C₁-C₇-alkyl, C₁-C₇-alkylsulfonylamino-C₁-C₇-alkyl, C₁-C₇-alkyl-NH—CO—NH—C₁-C₇-alkyl, C₁-C₇-alkyl-NH—SO₂—NH—C₁-C₇-alkyl, C₁-C₇-alkoxy, hydroxy-C₁-C₇-alkoxy, C₁-C₇-alkoxy-C₁-C₇-alkoxy, C₁-C₇-alkanoylamino-C₁-C₇-alkyloxy, carboxy-C₁-C₇-alkyloxy, C₁-C₇-alkyloxycarbonyl-C₁-C₇-alkoxy, mono- or di-(C₁-C₇-alkyl)-aminocarbonyl-C₁-C₇-alkyloxy, C₁-C₇-alkanoyloxy, mono- or di-(C₁-C₇-alkyl)-amino, mono-di-(naphthyl- or phenyl-C₁-C₇-alkyl)-amino, N-mono-C₁-C₇-alkoxy-C₁-C₇-alkylamino, C₁-C₇-alkanoylamino, C₁-C₇-alkylsulfonylamino, C₁-C₇-alkyl-carbonyl, halo-C₁-C₇-alkylcarbonyl, hydroxy-C₁-C₇-alkylcarbonyl, C₁-C₇-alkoxy-C₁-C₇-alkylcarbonyl, amino-C₁-C₇-alkylcarbonyl, (N—) mono- or (N,N—) di-(C₁-C₇-alkyl)-amino-C₁-C₇-alkylcarbonyl, C₁-C₇-alkanoylamino-C₁-C₇-alkylcarbonyl, C₁-C₇-alkoxy-carbonyl, hydroxy-C₁-C₇-alkoxycarbonyl, C₁-C₇-alkoxy-C₁-C₇-alkoxycarbonyl, amino-C₁-C₇-alkoxycarbonyl, (N—) mono-(C₁-C₇-alkyl)-amino-C₁-C₇-alkoxycarbonyl, C₁-C₇-alkanoylamino-C₁-C₇-alkoxycarbonyl, N-mono- or N,N-di-(C₁-C₇-alkyl)-aminocarbonyl, N—C₁-C₇-alkoxy-C₁-C₇-alkylcarbamoyl or N-mono- or N,N-di-(C₁-C₇-alkyl)-aminosulfonyl;

from C₂-C₇-alkenyl, C₂-C₇-alkynyl, phenyl, naphthyl, heterocyclyl, especially as defined below for heterocyclyl, preferably selected from pyrrolyl, furanyl, thienyl, thiazolyl, pyrazolyl, pyrazolidinonyl, N—(C₁-C₇-alkyl, phenyl, naphthyl, phenyl-C₁-C₇-alkyl or naphthyl-C₁-C₇-alkyl)-pyrazolidinonyl, triazolyl, tetrazolyl, oxetidinyl, 3-C₁-C₇-alkyl-oxetidinyl, pyridyl, pyrimidinyl, morpholino, piperidinyl, piperazinyl, pyrrolidinyl, tetrahydrofuran-onyl, tetrahydro-pyranyl, indolyl, indazolyl, 1H-indazolyl, benzofuranyl, benzothiophenyl, quinolinyl, isoquinolinyl, 1,2,3,4-tetrahydro-1,4-benzoxazinyl, 2H-1,4-benzoxazin-3(4H)-onyl, benzo[1,2,5]oxadiazolyl or 2H,3H-1,4-benzodioxinyl, phenyl- or naphthyl- or heterocyclyl-C₁-C₇-alkyl or —C₁-C₇-alkyloxy wherein heterocyclyl is as defined below, preferably selected from pyrrolyl, furanyl, thienyl, pyrimidinyl, pyrazolyl, pyrazolidinonyl, N—(C₁-C₇-alkyl, phenyl, naphthyl, phenyl-C₁-C₇-alkyl or naphthyl-C₁-C₇-alkyl)-pyrazolidinonyl, triazolyl, tetrazolyl, oxetidinyl, pyridyl, pyrimidinyl, morpholino, piperidinyl, piperazinyl, tetrahydrofuran-onyl, indolyl, indazolyl, 1H-indazanyl, benzofuranyl, benzothiophenyl, quinolinyl, isoquinolinyl, 1,2,3,4-tetrahydro-1,4-benzoxazinyl, 2H-1,4-benzoxazin-3(4H)-onyl- or benzo[1,2,5]oxadiazolyl; such as benzyl or naphthylmethyl, halo-C₁-C₇-alkyl, such as trifluoromethyl, phenyloxy- or naphthyloxy-C₁-C₇-alkyl, phenyl-C₁-C₇-alkoxy- or naphthyl-C₁-C₇-alkoxy-C₁-C₇-alkyl, di-(naphthyl- or phenyl)-amino-C₁-C₇-alkyl, di-(naphthyl- or phenyl-C₁-C₇-alkyl)-amino-C₁-C₇-alkyl, benzoyl- or naphthoylamino-C₁-C₇-alkyl, phenyl- or naphthylsulfonylamino-C₁-C₇-alkyl wherein phenyl or naphthyl is unsubstituted or substituted by one or more, especially one to three, C₁-C₇-alkyl moieties, phenyl- or naphthyl-C₁-C₇-alkylsulfonylamino-C₁-C₇-alkyl, carboxy-C₁-C₇-alkyl, halo, especially fluoro or chloro, hydroxy, phenyl-C₁-C₇-alkoxy wherein phenyl is unsubstituted or substituted by C₁-C₇-alkoxy and/or halo, halo-C₁-C₇-alkoxy, such as trifluoromethoxy, phenyl- or naphthyloxy, phenyl- or naphthyl-C₁-C₇-alkyloxy, phenyl- or naphthyl-oxy-C₁-C₇-alkyloxy, benzoyl- or naphthoyloxy, halo-C₁-C₇-alkylthio, such as trifluoromethylthio, phenyl- or naphthylthio, phenyl- or naphthyl-C₁-C₇-alkylthio, benzoyl- or naphthoylthio, nitro, amino, di-(naphthyl- or phenyl-C₁-C₇-alkyl)-amino, benzoyl- or naphthoylamino, phenyl- or naphthylsulfonylamino wherein phenyl or naphthyl is unsubstituted or substituted by one or more, especially one to three, C₁-C₇-alkoxy-C₁-C₇-alkyl or C₁-C₇-alkyl moieties, phenyl- or naphthyl-C₁-C₇-alkylsulfonylamino, carboxyl, (N,N—) di-(C₁-C₇-alkyl)-amino-C₁-C₇-alkoxycarbonyl, halo-C₁-C₇-alkoxycarbonyl, phenyl- or naphthyloxycarbonyl, phenyl- or naphthyl-C₁-C₇-alkoxycarbonyl, (N,N—) di-(C₁-C₇-alkyl)-amino-C₁-C₇-alkoxycarbonyl, carbamoyl, N-mono or N,N-di-(naphthyl-, phenyl-, C₁-C₇-alkyloxyphenyl and/or C₁-C₇-alkyloxynapthtyl-)aminocarbonyl, N-mono- or N,N-di-(naphthyl- or phenyl-C₁-C₇-alkyl)-aminocarbonyl, cyano, C₁-C₇-alkylene which is unsubstituted or substituted by up to four C₁-C₇-alkyl substituents and bound to two adjacent ring atoms of the aryl moiety, C₂-C₇-alkenylene or -alkynylene which are bound to two adjacent ring atoms of the aryl moiety, sulfenyl, sulfinyl, C₁-C₇-alkylsulfinyl, phenyl- or naphthylsulfinyl wherein phenyl or naphthyl is unsubstituted or substituted by one or more, especially one to three, C₁-C₇-alkoxy-C₁-C₇-alkyl or C₁-C₇-alkyl moieties, phenyl- or naphthyl-C₁-C₇-alkylsulfinyl, sulfonyl, C₁-C₇-alkylsulfonyl, halo-C₁-C₇-alkylsulfonyl, hydroxy-C₁-C₇-alkylsulfonyl, C₁-C₇-alkoxy-C₁-C₇-alkylsulfonyl, amino-C₁-C₇-alkylsulfonyl, (N,N—) di-(C₁-C₇-alkyl)-amino-C₁-C₇-alkylsulfonyl, C₁-C₇-alkanoylamino-C₁-C₇-alkylsulfonyl, phenyl- or naphthylsulfonyl wherein phenyl or naphthyl is unsubstituted or substituted by one or more, especially one to three, C₁-C₇-alkoxy-C₁-C₇-alkyl or C₁-C₇-alkyl moieties, phenyl- or naphthyl-C₁-C₇-alkylsulfonyl, sulfamoyl and N-mono or N,N-di-(C₁-C₇-alkyl, phenyl-, naphthyl, phenyl-C₁-C₇-alkyl and/or naphthyl-C₁-C₇-alkyl)-aminosulfonyl. Especially preferably aryl is phenyl or naphthyl, each of which is unsubstituted or substituted by one or more, e.g. up to three, substituents independently selected from the group consisting of C₁-C₇-alkyl, hydroxy-C₁-C₇-alkyl, C₁-C₇-alkoxy-C₁-C₇-alkyl, C₁-C₇-alkoxy-C₁-C₇-alkoxy-C₁-C₇-alkyl, amino-C₁-C₇-alkyl, C₁-C₇-alkoxy-C₁-C₇-alkylamino-C₁-C₇-alkyl, carboxy-C₁-C₇-alkyl, C₁-C₇-alkoxycarbonyl-C₁-C₇-alkyl, halo, especially fluoro, chloro or bromo, hydroxy, C₁-C₇-alkoxy, hydroxy-C₁-C₇-alkoxy C₁-C₇-alkoxy-C₁-C₇-alkoxy, amino-C₁-C₇-alkoxy, N—C₁-C₇-alkanoylamino-C₁-C₇-alkoxy, carboxyl-C₁-C₇-alkyloxy, C₁-C₇-alkoxycarbonyl-C₁-C₇-alkyloxy, carbamoyl-C₁-C₇-alkoxy, N-mono- or N,N-di-(C₁-C₇-alkyl)-carbamoyl-C₁-C₇-alkoxy, morpholino-C₁-C₇-alkoxy, pyridyl-C₁-C₇-alkoxy, amino, C₁-C₇-alkanoylamino, C₁-C₇-alkanoyl, C₁-C₇-alkoxy-C₁-C₇-alkanoyl, carboxy, carbamoyl, N—(C₁-C₇-alkoxy-C₁-C₇-alkyl)-carbamoyl, pyrazolyl, pyrazolyl-C₁-C₇-alkoxy, 4-C₁-C₇-alkylpiperidin-1-yl, nitro and cyano.

Unsubstituted or substituted heterocyclyl is preferably a mono- or bicyclic, unsaturated, partially saturated or saturated ring system with preferably 3 to 22 (more preferably 3 to 14) ring atoms and with one or more, preferably one to four, heteroatoms independently selected from nitrogen (═N—, —NH— or substituted —NH—), oxygen, sulfur (—S—, —S(═O)— or —S—(═O)₂—), and is unsubstituted or substituted by one or more, e.g. up to three, substitutents preferably independently selected from the substitutents mentioned above for aryl and from oxo. Preferably, heterocyclyl (which is unsubstituted or substituted as just mentioned) is selected from the following moieties (the asterisk marks the end of the bond binding to the rest of the molecule of formula I):

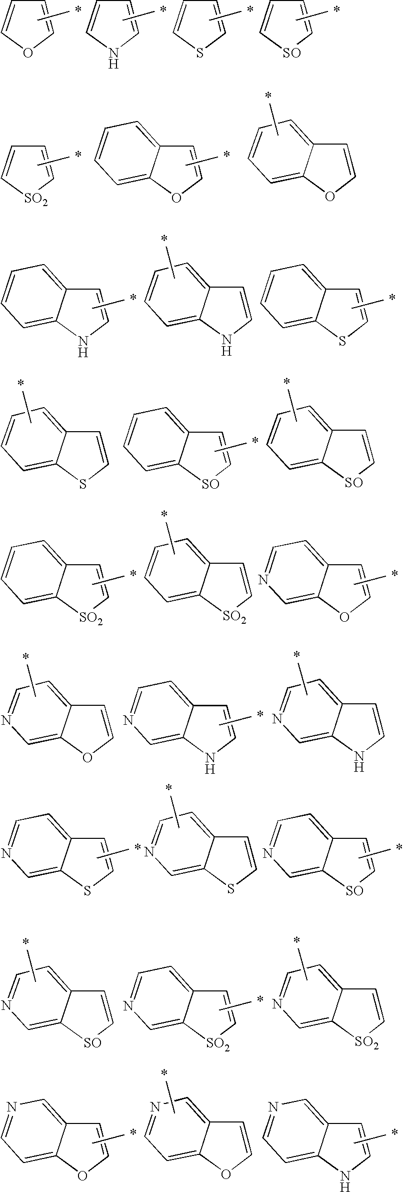

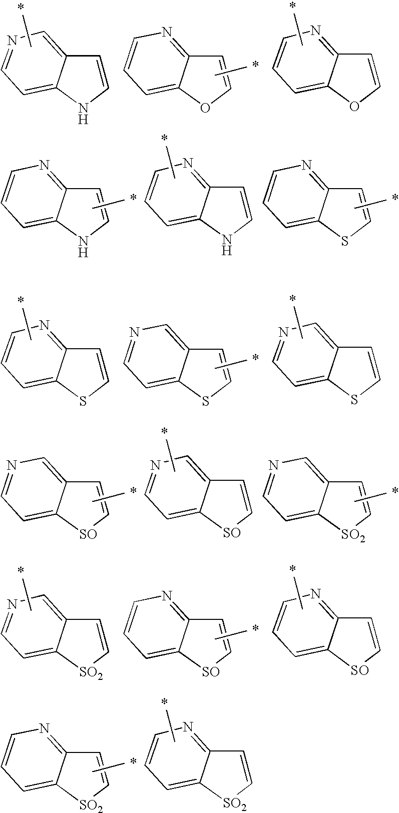

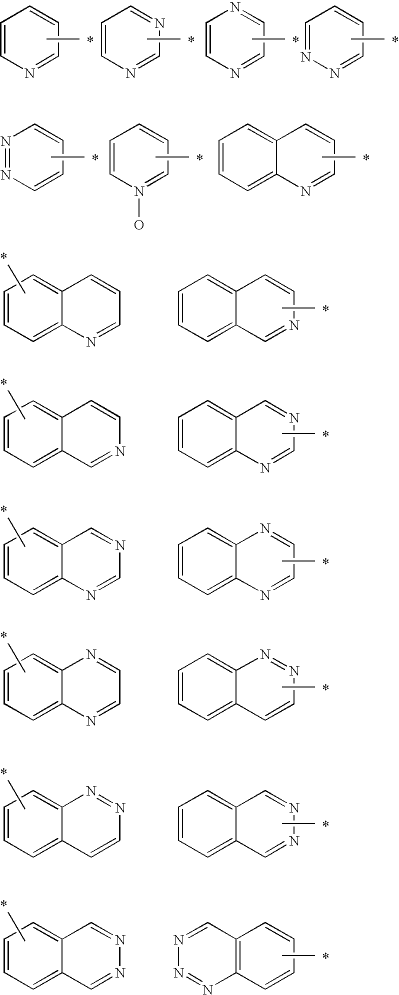

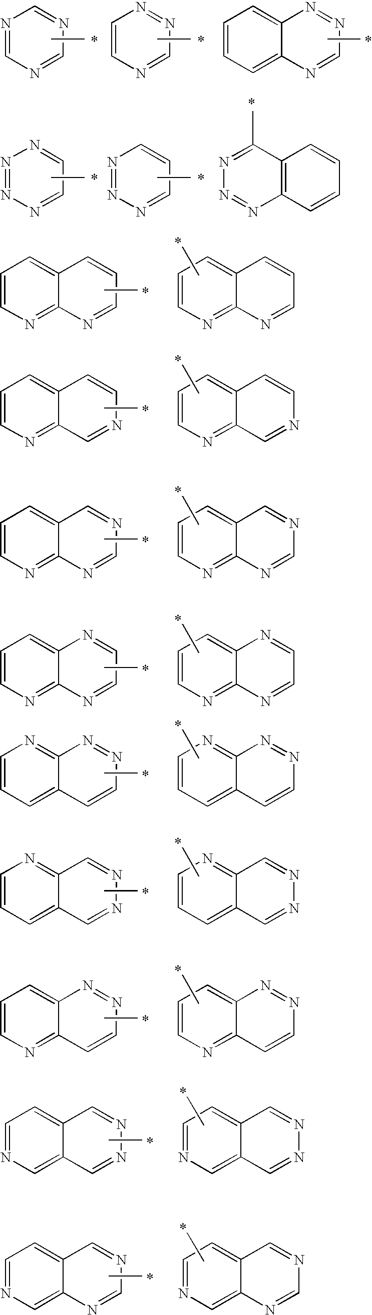

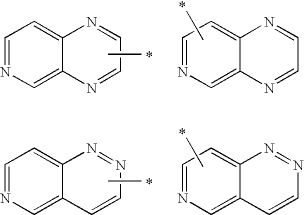

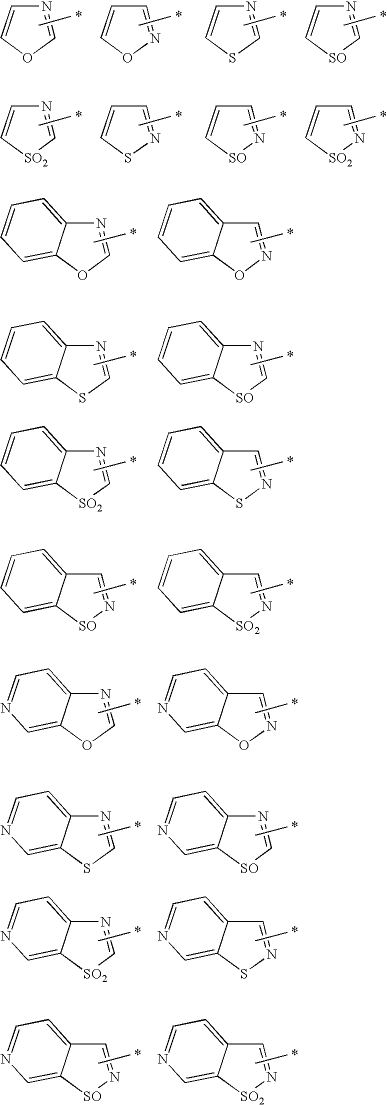

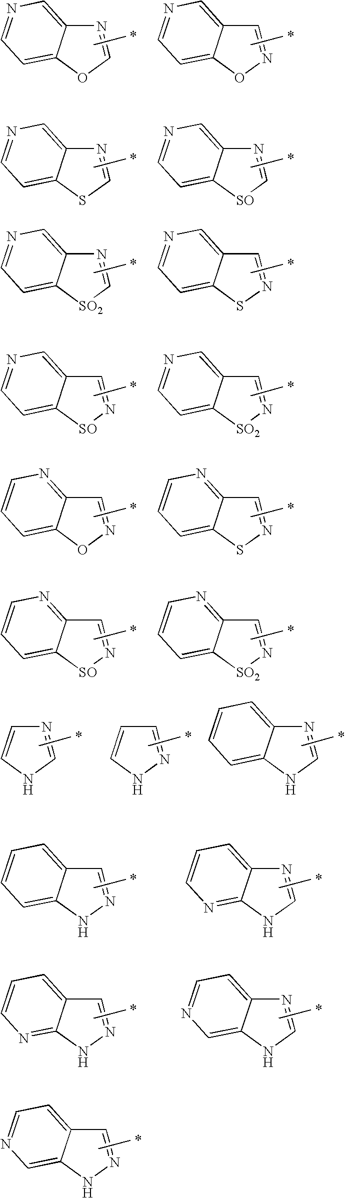

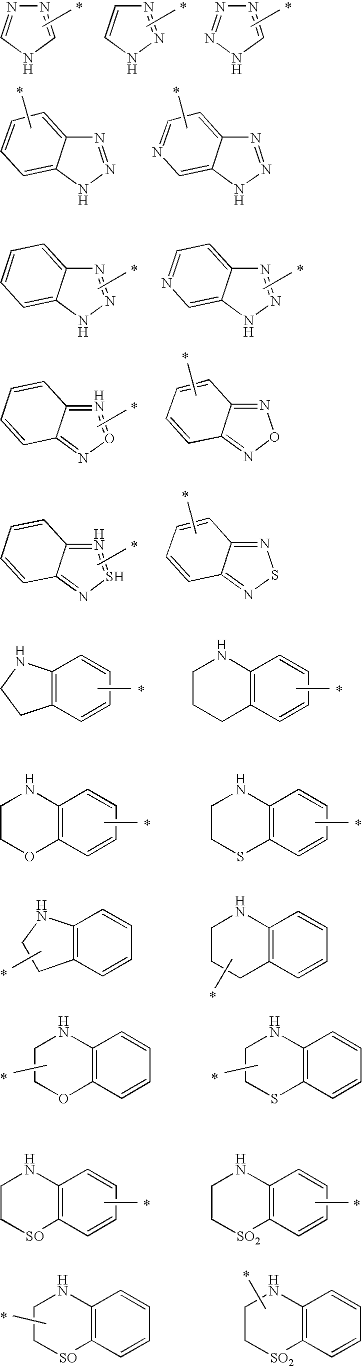

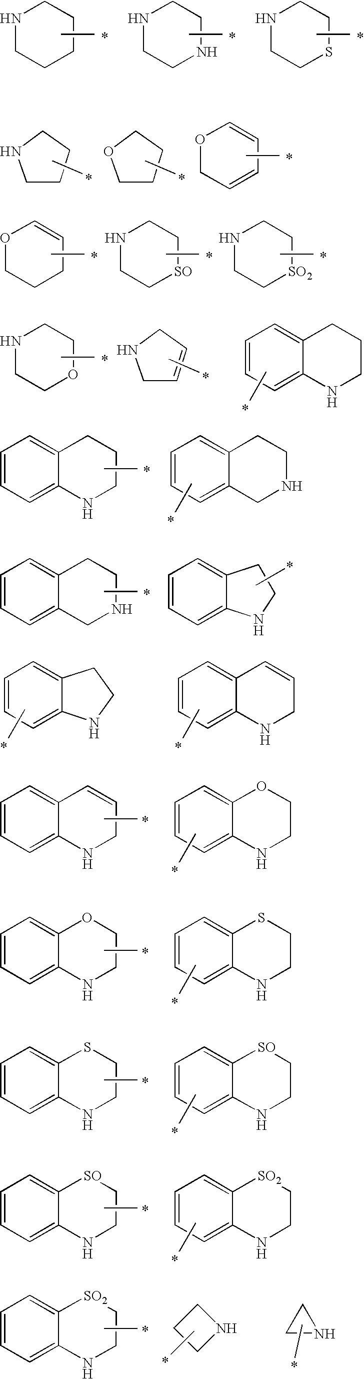

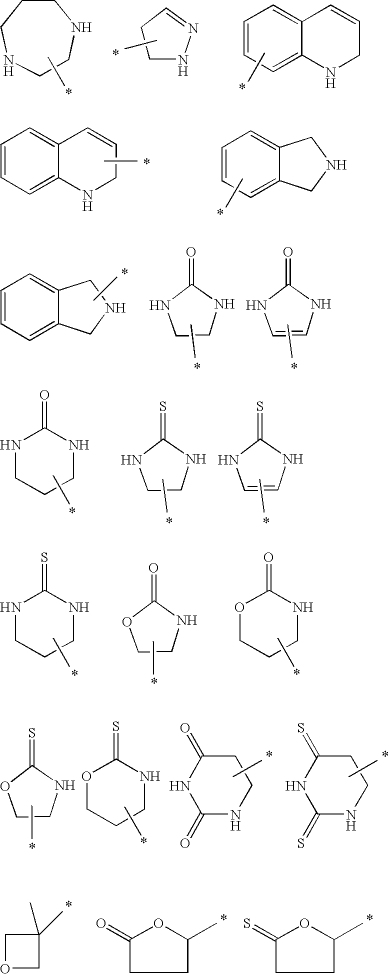

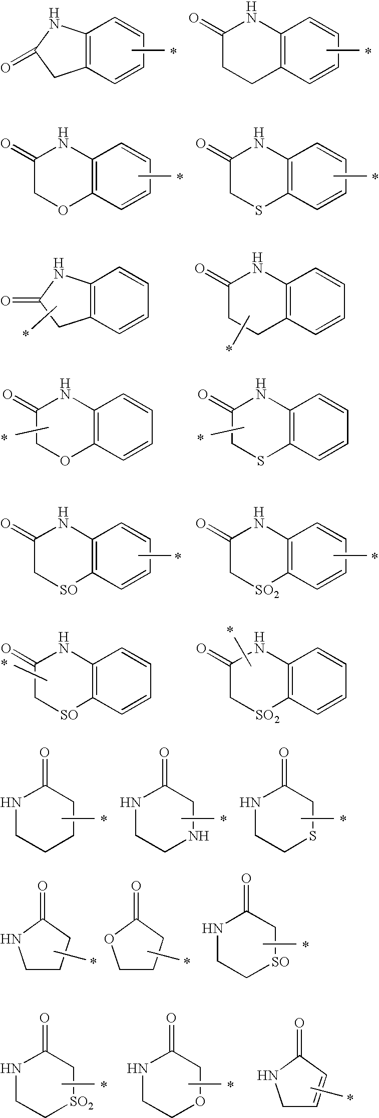

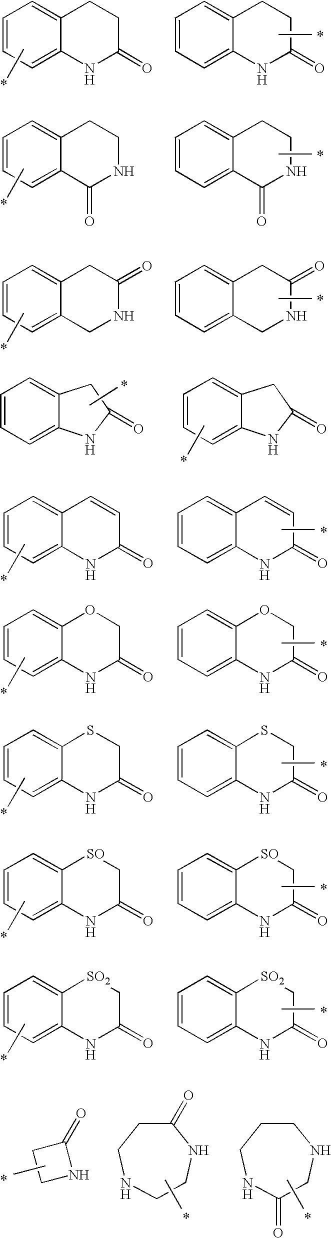

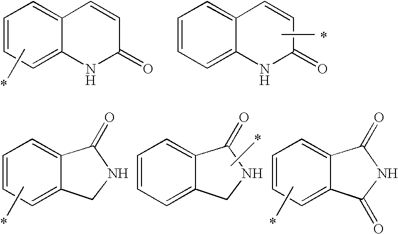

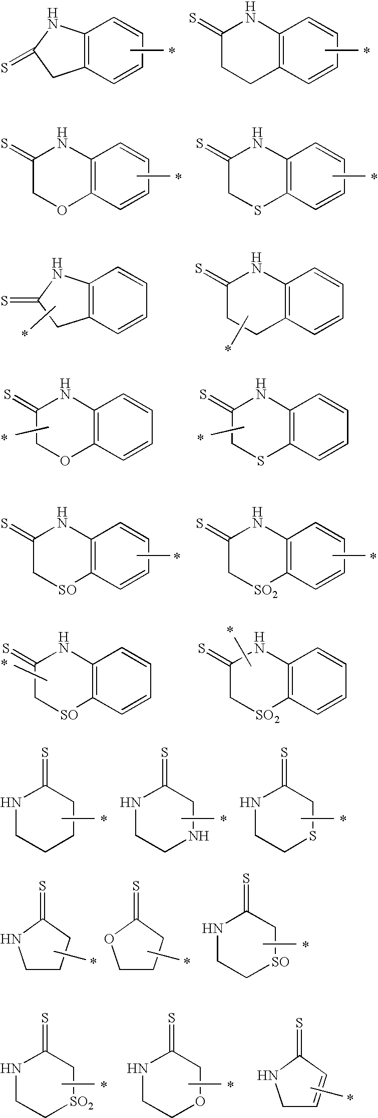

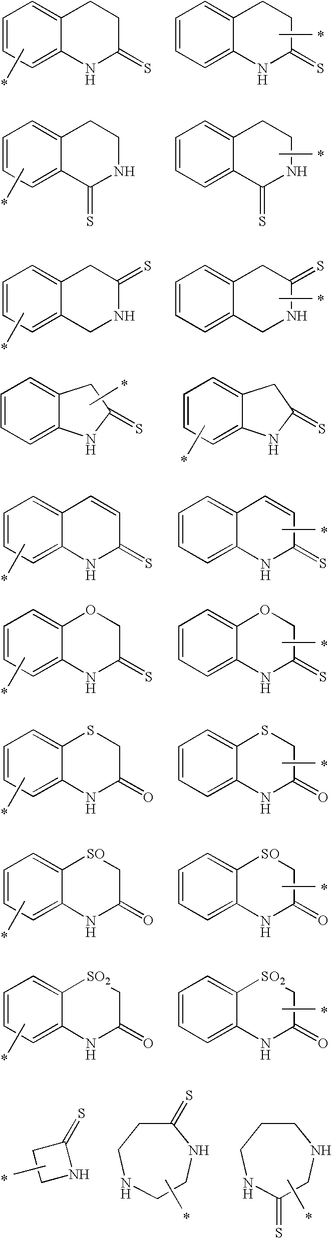

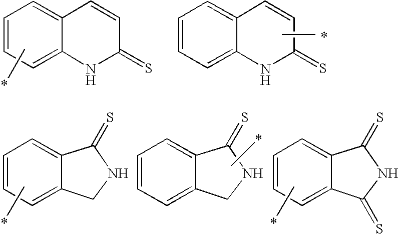

where in each case where an NH is present the bond with the asterisk connecting the respective heterocyclyl moiety to the rest of the molecule the H may be replaced with said bond and/or the H may be replaced by a substituent, preferably as defined above. Especially preferred as heterocyclyl is pyrrolyl, furanyl, thienyl, thiazolyl, pyrimidinyl, pyrazolyl, pyrazolidinonyl (=oxo-pyrazolidinyl), triazolyl, tetrazolyl, 1,3-oxazolyl, oxetidinyl, pyridyl, pyrimidinyl, morpholino, piperidinyl, piperazinyl, pyrrolidinyl, tetrahydrofuran-onyl (=oxo-tetrahydrofuranyl), tetrahydro-pyranyl, indolyl, indazolyl, 1H-indazanyl, benzofuranyl, benzo-thiophenyl, quinolinyl, isoquinolinyl, 1,2,3,4-tetrahydro-1,4-benzoxazinyl, 2H-1,4-benzoxazin-3(4H)-onyl, 2H,3H-1,4-benzodioxinyl, benzo[1,2,5]oxadiazolyl, pyridyl, indolyl, 1H-indazolyl, quinolyl, isoquinolyl or 1-benzothiophenyl; each of which is unsubstituted or substituted by one or more, e.g. up to three, substituents as mentioned above for substituted aryl, preferably independently selected from the group consisting of C₁-C₇-alkyl, hydroxy-C₁-C₇-alkyl, C₁-C₇-alkoxy-C₁-C₇-alkyl, C₁-C₇-alkoxy-C₁-C₇-alkoxy-C₁-C₇-alkyl, amino-C₁-C₇-alkyl, C₁-C₇-alkoxy-C₁-C₇-alkylamino-C₁-C₇-alkyl, carboxy-C₁-C₇-alkyl, C₁-C₇-alkoxy-C₁-C₇-alkyl, halo, hydroxy, C₁-C₇-alkoxy, C₁-C₇-alkoxy-C₁-C₇-alkoxy, amino-C₁-C₇-alkoxy, N—C₁-C₇-alkanoylamino-C₁-C₇-alkoxy, carbamoyl-C₁-C₇-alkoxy, N—C₁-C₇-alkylcarbamoyl-C₁-C₇-alkoxy, C₁-C₇-alkanoyl, C₁-C₇-alkoxy-C₁-C₇-alkanoyl, carboxy, carbamoyl and N—C₁-C₇-alkoxy-C₁-C₇-alkylcarbamoyl. In the case of heterocycles including an NH ring member, the substitutents, as far as bound via a carbon or oxygen atom, are preferably bound at the nitrogen instead of the H.

Unsubstituted or substituted cycloalkyl is preferably mono- or polycyclic, more preferably monocyclic, C₃-C₁₀-cycloalkyl which may include one or more double (e.g. in cycloalkenyl) and/or triple bonds (e.g. in cycloalkynyl), and is unsubstituted or substituted by one or more, e.g. one to three substitutents preferably independently selected from those mentioned above as substituents for aryl. Preferred is cyclopropyl, cyclobutyl, cyclopentyl, cyclohexyl or cycloheptyl.

Acyl is preferably unsubstituted or substituted aryl-carbonyl or -sulfonyl, unsubstituted or substituted heterocyclylcarbonyl or -sulfonyl, unsubstituted or substituted cycloalkylcarbonyl or -sulfonyl, formyl or unsubstituted or substituted alkylcarbonyl or -sulfonyl, or (especially if G is oxy or preferably if it is NR8, especially imino (NH)) in the case of acyl R5) unsubstituted or substituted alkyloxycarbonyl or -oxysulfonyl, unsubstituted or substituted aryl-oxycarbonyl or -oxysulfonyl, unsubstituted or substituted heterocyclyloxycarbonyl or -oxysulfonyl, unsubstituted or substituted cycloalkyloxycarbonyl or -oxysulfonyl or N-mono- or N,N-di-(unsubstituted or substituted aryl, unsubstituted or substituted heterocyclyl, unsubstituted or substituted cycloalkyl or unsubstituted or substituted alkyl)-aminocarbonyl, with the proviso that in cases of -oxycarbonyl bound moieties G is NR8, preferably NH; wherein unsubstituted or substituted aryl, unsubstituted or substituted heterocyclyl, unsubstituted or substituted cycloalkyl and unsubstituted or substituted alkyl are preferably as described above. Preferred is C₁-C₇-alkanoyl, such as acetyl, 3,3-dimethyl-butyryl, 2,2-dimethyl-propionyl or 3,3-dimethyl-butyryl, unsubstituted or mono-, di- or tri-(halo and/or C₁-C₇-alkyl)-substituted benzoyl or naphthoyl, such as 4-methyl-benzoyl, C₃-C₈-cycloalkylcarbonyl, such as cyclobutylcarbonyl, unsubstituted or phenyl-substituted pyrrolidinylcarbonyl, especially phenyl-pyrrolidinocarbonyl, C₁-C₇-alkylsulfonyl, such as methylsulfonyl (=methanesulfonyl), (phenyl- or naphthyl)-C₁-C₇-alkylsulfonyl, such as phenylmethanesulfonyl, or (unsubstituted, or [C₁-C₇-alkyl-, phenyl-, halo-lower alkyl-, halo, oxo-C₁-C₇-alkyl-C₁-C₇-alkyloxy-, phenyl-C₁-C₇-alkoxy-, halo-C₁-C₇-alkyloxy-, phenoxy-, C₁-C₇-alkanoylamino-, cyano-, C₁-C₇-alkanoyl- and/or C₁-C₇-alkylsulfonyl-]substituted) (phenyl-or naphthyl)-sulfonyl, such as phenylsulfonyl (=benzenesulfonyl), naphthalene-1-sulfonyl, naphthalene-2-sulfonyl, toluene-4-sulfonyl, 4-isopropyl-benzenesulfonyl, biphenyl-4-sulfonyl, 2-trifluoromethyl-benzenesulfonyl, 4-chloro-benzenesulfonyl, 3-chloro-benzenesulfonyl, 2-chloro-benzenesulfonyl, 2,4-difluoro-benzenesulfonyl, 2,6-difluoro-benzenesulfonyl, 2,5-dichloro-benzenesulfonyl, 3,4-dichloro-benzenesulfonyl, 3,5-dichloro-benzenesulfonyl, 2,3-dichloro-benzenesulfonyl, 3-methoxy-benzenesulfonyl, 4-methoxy-benzenesulfonyl, 2,5-dimethoxy-benzenesulfonyl, 4-trifluoromethoxy-benzenesulfonyl, 2-benzyloxy-benzenesulfonyl, 3-trifluoromethyl-benzenesulfonyl, 4-phenoxy-benzenesulfonyl, 4-(2-oxo-propyl)-benzenesulfonyl, 4-acetylamino-benzenesulfonyl, 4-cyano-benzenesulfonyl, 2-cyano-benzenesulfonyl, 3-cyano-benzenesulfonyl, 3-acetyl-benzenesulfonyl or 4-methanesulfonyl-benzenesulfonyl, halo-thiophene-2-sulfonyl, such as 5-chloro-thiophene-2-sulfonyl, quinoline-sulfonyl, such as quinoline-8-sulfonyl, (C₁-C₇-alkanoylamino and/or C₁-C₇-alkyl)-substituted thiazol-sulfonyl, such as 2-acetylamino-4-methyl-thiazole-5-sulfonyl, (halo and/or C₁-C₇-alkyl)-substituted pyrazolesulfonyl, such as 5-chloro-1,3-dimethyl-1H-pyrazole-4-sulfonyl, pyridine-sulfonyl, such as pyridine-3-sulfonyl, or N-mono- or N,N-di-(C₁-C₇-alkyl, (unsubstituted or halo-substituted) phenyl or naphthyl, phenyl-C₁-C₇-alkyl, naphthyl-C₁-C₇-alkyl or C₃-C₈-cycloalkyl)-aminocarbonyl, such as N-tert-butyl-aminocarbonyl, (3-chloro-phenyl)-aminocarbonyl, N-benzyl-aminocarbonyl, N-cyclohexyl-aminocarbonyl, C₁-C₇-alkylaminocarbonyl or phenyl-C₁-C₇alkylaminocarbonyl, or (C₁-C₇-alkyl, phenyl, naphthyl, phenyl-C₁-C₇-alkyl and/or napthyl-C₁-C₇-alkyl)-oxycarbonyl, e.g. C₁-C₇-alkoxycarbonyl, such as tert-butyloxycarbonyl or isobutyloxycarbonyl, or phenyl-C₁-C₇-alkyloxycarbonyl.

“-Oxycarbonyl-” means —O—C(═O)—, “aminocarbonyl” means in the case of mono-substitution —NH—C(═O)—, in the case of double substitution also the second hydrogen is replaced by the corresponding moiety. For example, C₁-C₇-alkoxycarbonyl is C₁-C₇-alkyl-O—C(═O)—.

Etherified or esterified hydroxy is especially hydroxy that is esterified with acyl as defined above, especially in C₁-C₇-alkanoyloxy; or preferably etherified with alkyl, alkenyl, alkynyl, aryl, heterocyclyl or cycloalkyl each of which is unsubstituted or substituted and is preferably as described above for the corresponding unsubstituted or substituted moieties. Especially preferred is

unsubstituted or especially substituted C₁-C₇-alkyloxy, especially with a substituent selected from C₁-C₇-alkoxy; phenyl, tetrazolyl, tetrahydrofuran-onyl, oxetidinyl, 3-(C₁-C₇-alkyl)-oxetidinyl, pyridyl or 2H,3H-1,4-benzodioxinyl, each of which is unsubstituted or substituted by one or more, preferably up to three, e.g. 1 or two substituents independently selected from C₁-C₇-alkyl, hydroxy, C₁-C₇-alkoxy, phenyloxy wherein phenyl is unsubstituted or substituted, preferably up to three times, by C₁-C₇-alkoxy and/or halo, phenyl-C₁-C₇-alkoxy wherein phenyl is unsubstituted or substituted, preferably up to three times, by C₁-C₇-alkoxy and/or halo; halo, amino, N-mono- or N,N-di(C₁-C₇-alkyl, phenyl, naphthyl, phenyl-C₁-C₇-alkyl or naphthyl-C₁-C₇-alkyl)amino, C₁-C₇-alkanoylamino, carboxy, N-mono- or N,N-di(C₁-C₇-alkyl, phenyl, naphthyl, phenyl-C₁-C₇-alkyl or naphthyl-C₁-C₇-alkyl)-aminocarbonyl, morpholino, morpholino-C₁-C₇-alkoxy, pyridyl-C₁-C₇-alkoxy, pyrazolyl, 4-C₁-C₇-alkylpiperidin-1-yl and cyano; or selected from morpholino;

or unsubstituted or substituted aryloxy with unsubstituted or substituted aryl as described above, especially phenyloxy with phenyl that is unsubstituted or substituted, preferably up to three times, by C₁-C₇-alkoxy and/or halo; or

unsubstituted or substituted heterocyclyloxy with unsubstituted or substituted heterocyclyl as described above, preferably tetrahydropyranyloxy.

Substituted mercapto can be mercapto that is thioesterified with acyl as defined above, especially with lower alkanoyloxy; or preferably thioetherified with alkyl, alkenyl, alkynyl, aryl, heterocyclyl or cycloalkyl each of which is unsubstituted or substituted and is preferably as described above for the corresponding unsubstituted or substituted moieties. Especially preferred is unsubstituted or especially substituted C₁-C₇-alkylthio or unsubstituted or substituted arylthio with unsubstituted or substituted C₁-C₇-alkyl or aryl as just described for the corresponding moieties under etherified hydroxy.

Substituted sulfinyl or sulfonyl can be substituted with alkyl, alkenyl, alkynyl, aryl, heterocyclyl or cycloalkyl each of which is unsubstituted or substituted and is preferably as described above for the corresponding unsubstituted or substituted moieties. Especially preferred is unsubstituted or especially substituted C₁-C₇-alkylsulfinyl or -sulfonyl or unsubstituted or substituted arylsulfinyl or -sulfonyl with unsubstituted or substituted C₁-C₇-alkyl or aryl as just described for the corresponding moieties under etherified hydroxy.

In mono- or di-substituted amino, amino is preferably substituted by one or more substituents selected from one acyl, especially C₁-C₇-alkanoyl, phenylcarbonyl (=benzoyl), C₁-C₇-alkylsulfonyl or phenylsulfonyl wherein phenyl is unsubstituted or substituted by one to 3 C₁-C₇-alkyl groups, and from one or two moieties selected from alkyl, alkenyl, alkynyl, aryl, heterocyclyl and cycloalkyl each of which is unsubstituted or substituted and is preferably as described above for the corresponding unsubstituted or substituted moieties. Preferred is C₁-C₇-alkanoylamino, mono- or di-(phenyl, naphthyl, C₁-C₇-alkoxy-phenyl, C₁-C₇-alkoxynaphthyl, naphthyl-C₁-C₇-alkyl or phenyl-C₁-C₇-alkyl)-carbonylamino (e.g. 4-methoxybenzoylamino), mono- or di-(C₁-C₇-alkyl and/or C₁-C₇-alkoxy-C₁-C₇-alkyl)-amino or mono- or di-(phenyl, naphthyl, C₁-C₇-alkoxy-phenyl, C₁-C₇-alkoxynaphthyl, phenyl-C₁-C₇-alkyl, naphthyl-C₁-C₇-alkyl, C₁-C₇-alkoxy-naphthyl-C₁-C₇-alkyl or C₁-C₇-alkoxy-phenyl-C₁-C₇-alkyl)-amino.

Esterified carboxy is preferably alkyloxycarbonyl, aryloxycarbonyl, heterocyclyloxycarbonyl or cycloalkyloxycarbonyl, wherein alkyl, aryl, heterocyclyl and cycloalkyl are unsubstituted or substituted and the corresponding moieties and their substituents are preferably as described above. Preferred is C₁-C₇-alkoxycarbonyl, phenyl-C₁-C₇-alkyloxycarbonyl, phenoxycarbonyl, or naphthoxycarbonyl.

In amidated carboxy, the amino part bound to the carbonyl in the amido function (D₂N—C(═O)—) wherein each D is independently of the other hydrogen or an amino substituent) is unsubstituted or substituted as described for substituted amino, but preferably without acyl as amino substituent. Preferred is mono- or di-(C₁-C₇-alkyl and/or C₁-C₇-alkoxy-C₁-C₇alkyl)-aminocarbonyl or mono- or di-(C₁-C₇-alkyloxyphenyl, C₁-C₇-alkyloxynaphthyl, naphthyl-C₁-C₇-alkyl or phenyl-C₁-C₇-alkyl)-aminocarbonyl.

In substituted sulfamoyl, the amino part bound to the sulfonyl in the sulfamoyl function (D₂N—S(═O)₂—) wherein each D is independently of the other hydrogen or an amino substituent) is unsubstituted or substituted as described for substituted amino, but preferably without acyl as amino substituent. Preferred is mono- or di-(C₁-C₇-alkyl and/or C₁-C₇-alkoxy-C₁-C₇alkyl)-aminosulfonyl or mono- or di-(C₁-C₇-alkyloxyphenyl, C₁-C₇-alkyloxynaphthyl, naphthyl-C₁-C₇-alkyl or phenyl-C₁-C₇-alkyl)-aminosulfonyl.

Unsubstituted or substituted C₁-C₇-alkyl, unsubstituted or substituted C₂-C₇-alkenyl and unsubstituted or substituted C₂-C₇-alkynyl and their substituents are defined as above under the corresponding (un)substituted alkyl, (un)substituted alkynyl and (un)substituted alkynyl moieties but with the given number of carbon atoms in the alkyl, alkenyl or alkynyl moieties.

The following preferred embodiments of the moieties and symbols in formula I can be employed independently of each other to replace more general definitions and thus to define specially preferred embodiments of the invention, where the remaining definitions can be kept broad as defined in embodiments of the inventions defined above of below.

Preferred Definitions for G, R5 and R6

As G, oxy, imino (NH) or NR8 wherein R8 is C₁-C₇-alkyl or (unsubstituted or halo)-phenyl-C₁-C₇-alkyl are preferred.

As R5 hydrogen, unsubstituted C₁-C₇-alkyl or C₁-C₇-alkyl substituted with up to three hydroxy groups, or C₁-C₇-alkyl, preferably methyl or ethyl, substituted with one or two, preferably one carboxy or aminocarbonyl group, (unsubstituted or halo)-phenyl-C₁-C₇-alkyl, C₁-C₇-alkanoyl, such as 3,3-dimethyl-butyryl, 2,2-dimethyl-propionyl or 3,3-dimethyl-butyryl, unsubstituted or mono-, di- or tri-(halo and/or C₁-C₇-alkyl)-substituted benzoyl or naphthoyl, such as 4-methyl-benzoyl, C₃-C₈-cycloalkylcarbonyl, such as cyclobutylcarbonyl, unsubstituted or phenyl-substituted pyrrolidinylcarbonyl, especially phenyl-pyrrolidinocarbonyl, C₁-C₇-alkylsulfonyl, such as methylsulfonyl (=methanesulfonyl), (phenyl- or naphthyl)-C₁-C₇-alkylsulfonyl, such as phenylmethanesulfonyl, or (unsubstituted, or [C₁-C₇-alkyl-, phenyl-, halo-lower alkyl-, halo, oxo-C₁-C₇-alkyl-C₁-C₇-alkyloxy-, phenyl-C₁-C₇-alkoxy-, halo-C₁-C₇-alkyloxy-, phenoxy-, C₁-C₇-alkanoylamino-, cyano-, C₁-C₇-alkanoyl- and/or C₁-C₇-alkylsulfonyllsubstituted) (phenyl- or naphthyl)-sulfonyl, such as naphthalene-1-sulfonyl, naphthalene-2-sulfonyl, toluene-4-sulfonyl, 4-isopropyl-benzenesulfonyl, biphenyl-4-sulfonyl, 2-trifluoromethyl-benzenesulfonyl, 4-chloro-benzenesulfonyl, 3-chloro-benzenesulfonyl, 2-chloro-benzenesulfonyl, 2,4-difluoro-benzenesulfonyl, 2,6-difluoro-benzenesulfonyl, 2,5-dichloro-benzenesulfonyl, 3,4-dichloro-benzenesulfonyl, 3,5-dichloro-benzenesulfonyl, 2,3-dichloro-benzenesulfonyl, 3-methoxy-benzenesulfonyl, 4-methoxy-benzenesulfonyl, 2,5-dimethoxy-benzenesulfonyl, 4-trifluoromethoxy-benzenesulfonyl, 2-benzyloxy-benzenesulfonyl, 3-trifluoromethyl-benzenesulfonyl, 4-phenoxy-benzenesulfonyl, 4-(2-oxo-propyl)-benzenesulfonyl, 4-acetylamino-benzenesulfonyl, 4-cyano-benzenesulfonyl, 2-cyano-benzenesulfonyl, 3-cyano-benzenesulfonyl, 3-acetyl-benzenesulfonyl, 4-methanesulfonyl-benzenesulfonyl, halo-thiophene-2-sulfonyl, such as 5-chloro-thiophene-2-sulfonyl, quinoline-sulfonyl, such as quinoline-8-sulfonyl, (C₁-C₇-alkanoylamino and/or C₁-C₇-alkyl)-substituted thiazol-sulfonyl, such as 2-acetylamino-4-methyl-thiazole-5-sulfonyl, (halo and/or C₁-C₇-alkyl)-substituted pyrazolesulfonyl, such as 5-chloro-1,3-dimethyl-1H-pyrazole-4-sulfonyl, pyridine-sulfonyl, such as pyridine-3-sulfonyl, N-mono- or N,N-di-(C₁-C₇-alkyl, (unsubstituted or halo-substituted) phenyl or naphthyl, phenyl-C₁-C₇-alkyl, naphthyl-C₁-C₇-alkyl or C₃-C₈-cycloalkyl)-aminocarbonyl, such as N-tert-butyl-aminocarbonyl, (3-chloro-phenyl)-aminocarbonyl, N-benzyl-aminocarbonyl, N-cyclohexyl-aminocarbonyl, or (C₁-C₇-alkyl, phenyl, naphthyl, phenyl-C₁-C₇-alkyl and/or napthyl-C₁-C₇-alkyl)-oxycarbonyl, e.g. C₁-C₇-alkoxycarbonyl, such as tert-butyloxycarbonyl or isobutyloxycarbonyl, or phenyl-C₁-C₇-alkyloxycarbonyl are preferred; or G is methylene and R5 is phenyl, C₁-C₇-alkoxy-C₁-C₇-alkyl, hydroxy, C₁-C₇-alkoxy-C₁-C₇-alkoxy-C₁-C₇-alkyl, C₁-C₇-alkoxy or C₁-C₇-alkoxy-C₁-C₇-alkoxy; or G-R5 is halo, especially fluoro, especially if R6 is halo, preferably fluoro. More preferably, R5 is hydrogen, unsubstituted C₁-C₇-alkyl, or C₁-C₇-alkyl substituted with up to three, e.g. one, hydroxy groups, or C₁-C₇-alkyl, preferably methyl or ethyl, substituted with one or two, preferably one carboxy or aminocarbonyl group, N-mono- or N,N-di-(C₁-C₇-alkyl, (unsubstituted or halo-substituted) phenyl or naphthyl, phenyl-C₁-C₇-alkyl, naphthyl-C₁-C₇-alkyl or C₃-C₈-cycloalkyl)-aminocarbonyl, C₁-C₇-alkylsulfonyl, such as methylsulfonyl, C₁-C₇-alkanoyl, or G is methylene and R5 is phenyl, C₁-C₇-alkoxy-C₁-C₇-alkyl, hydroxy or C₁-C₇-alkoxy, or G-R5 is halo, especially fluoro.

Preferred examples for G-R5 are as shown below:

OH, F, OMe, OC(O)NHEt, NH₂, CH₂OH, CH₂OMe,

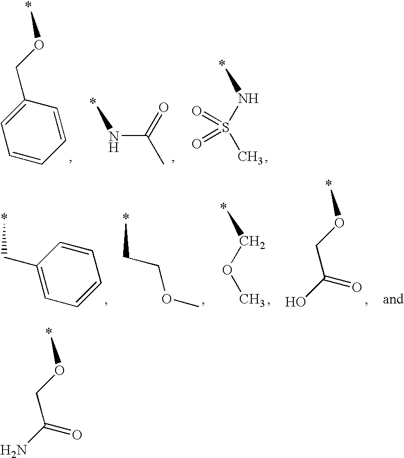

or the antipode thereof.

Where it is mentioned that or G-R5 and R6 together are oxo (═O) and/or G-R5 is hydroxy and R6 is hydroxy or where oxo can be present also in the hydrated form as two hydroxy groups, this is intended to mean that the corresponding compounds (as well as precursors thereof) may be present in the keto form (C═O), the hydrated (C(OH)₂) form or a mixture thereof (resulting from an equilibrium). Compounds of the formula I wherein G-R5 and R6 have other meanings mentioned hereinbefore or hereinafter than those wherein G-R5 and R6 together are oxo and/or G-R5 is hydroxy and R6 is hydroxy are preferred over those wherein wherein G-R5 and R6 together are oxo and/or G-R5 is hydroxy and R6 is hydroxy.

R6 is as defined herein, preferably hydrogen, halogen, such as F, OH or C₁-C₇-alkyl, such as methyl, more preferably hydrogen, halogen or methyl When R6 is halogen, G-R5 are preferably also halogen. Most preferably in this embodiment, both R6 and G-R5 are F. When R6 is OH or C₁-C₇-alkyl, such as methyl, G-R5 is preferably also OH.

Preferred Definitions for R1

As R1, C₁-C₇-alkyl, halo-C₁-C₇-alkyl, e.g. 3,3,3-trifluoroethyl or C₃-C₈-cyclopropyl is especially preferred. R1 is more preferably C₃-C₈-cycloalkyl, still more preferably C₃-, C₄-, C₅- or C₆-cycloalkyl, most preferably cyclopropyl.

Preferred Definitions for R2

R2 preferably has one of the meanings given for R2 herein other than acyl. More especially, R2 is unsubstituted or substituted alkyl, unsubstituted or substituted aryl or unsubstituted or substituted heterocyclyl.

In a first embodiment R2 is preferably unsubstituted or substituted alkyl.

Preferred examples for alkyl are branched or straight chain C₁-C₇-alkyl which may be substituted or unsubstituted. Preferred examples include methyl, ethyl, isopropyl, n-propyl, n-butyl, sec-butyl or tert-butyl, more preferably methyl, ethyl or isopropyl, most preferably methyl. The alkyl moiety is preferably substituted. When the alkyl moiety is substituted, it is preferably mono-, di- or tri-substituted, more preferably mono-substituted. Suitable substituents for the alkyl moiety are as defined herein, preferably O—C₁-C₄-alkyl, halo, hydroxy, unsubstituted or substituted, preferably substituted, phenyl, unsubstituted or substituted, preferably substituted, naphthyl, unsubstituted or substituted, preferably substituted, phenyl- or naphthyloxy, unsubstituted or substituted, preferably substituted, phenyl- or naphthyl-C₁-C₇-alkyloxy, unsubstituted or substituted, preferably substituted, heterocyclycl, unsubstituted or substituted, preferably unsubstituted, cycloalkyl, nitro, amino, amino-C₁-C₇-alkyl, N-mono- or N,N-di-substituted aminocarbonyl, carboxyl, and cyano, more preferably unsubstituted or substituted, preferably substituted, phenyl, unsubstituted or substituted, preferably substituted, naphthyl, unsubstituted or substituted, preferably substituted, phenyl- or naphthyloxy, or unsubstituted or substituted, preferably substituted, heterocyclycl. The heterocyclyl moietyl is in this connection preferably mono- or bicyclic. Preferred are aromatic ring systems, or in particular if a bicyclic moiety is contemplated, partially saturated ring systems, in particular whereby one of the rings is aromatic and the other is saturated or partially saturated, most preferred are aromatic. The heterocyclyl moiety has preferably 1, 2 or 3, more preferably 1 or 2, most preferably 1, heteroatoms selected from O, N or S, more preferably S or N. Particularly preferred examples include 6-membered rings preferably containing a nitrogen atom, in particular pyridyl; or bicyclic ring systems preferably containing a N or S atom, in particular indolyl, 1H-indazolyl, quinolyl, isoquinolyl, 1,2,3,4-tetrahydro-1,4-benzoxazinyl, 2H-1,4-benzoxazin-3(4H)-onyl, 9-xanthenyl, or 1-benzothiophenyl, where each moiety mentioned above as being substituted, in particular phenyl, naphthyl, pyridyl, indolyl, 1H-indazolyl, quinolyl, isoquinolyl, 1,2,3,4-tetrahydro-1,4-benzoxazinyl, 2H-1,4-benzoxazin-3(4H)-onyl or 1-benzothiophenyl is unsubstituted or substituted by one or more, e.g. up to three, substituents independently selected from the group consisting of C₁-C₇-alkyl, hydroxy-C₁-C₇-alkyl, C₁-C₇-alkoxy-C₁-C₇-alkyl, C₁-C₇-alkoxy-C₁-C₇-alkoxy-C₁-C₇-alkyl, C₁-C₇-alkanoyloxy-C₁-C₇-alkyl, amino-C₁-C₇-alkyl, C₁-C₇-alkoxy-C₁-C₇-alkylamino-C₁-C₇-alkyl, C₁-C₇-alkanoylamino-C₁-C₇-alkyl, C₁-C₇-alkylsulfonylamino-C₁-C₇-alkyl, carboxy-C₁-C₇-alkyl, C₁-C₇-alkoxycarbonyl-C₁-C₇-alkyl, halo, hydroxy, C₁-C₇-alkoxy, C₁-C₇-alkoxy-C₁-C₇-alkoxy, carboxy-C₁-C₇-alkoxy, amino-C₁-C₇-alkoxy, N—C₁-C₇-alkanoylamino-C₁-C₇-alkoxy, carbamoyl-C₁-C₇-alkyl, carbamoyl-C₁-C₇-alkoxy, N—C₁-C₇-alkylcarbamoyl-C₁-C₇-alkoxy, C₁-C₇-alkanoyl, C₁-C₇-alkyloxy-C₁-C₇-alkanoyl, C₁-C₇-alkoxy-C₁-C₇-alkanoyl, carboxyl, carbamoyl and N—C₁-C₇-alkoxy-C₁-C₇-alkylcarbamoyl, more preferably C₁-C₇-alkyl, hydroxy-C₁-C₇-alkyl, C₁-C₇-alkoxy-C₁-C₇-alkyl, halo, C₁-C₇-alkoxy, C₁-C₇-alkoxy-C₁-C₇-alkoxy, carboxy-C₁-C₇-alkoxy, and, carbamoyl-C₁-C₇-alkyl, in particular methyl, hydroxy-propyl, hydroxyl-butyl, methoxy-propyl, Cl, F, methoxy, methoxy-propyloxy, carboxy-ethyloxy and, carbamoyl-propyl. The heterocyclyl moiety is preferably substituted on the N, if present.

In a second embodiment R2 is preferably unsubstituted or substituted aryl.

Preferred examples of aryl include phenyl or naphthyl, more preferably phenyl. When the aryl moiety is substituted, it is preferably mono- or di-substituted. Most preferably aryl is di-substituted. Suitable substituents are as defined herein, preferably C₁-C₇-alkyl, —O—C₁-C₇-alkyl, halo-C₁-C₇-alkyl, —O-halo-C₁-C₇-alkyl, halo, hydroxy, nitro, amino, amino-C₁-C₇-alkyl, carboxyl, cyano, hydroxy-C₁-C₇-alkyl, C₁-C₇-alkoxy-C₁-C₇-alkyl, C₁-C₇-alkoxy-C₁-C₇-alkoxy-C₁-C₇-alkyl, C₁-C₇-alkoxy-C₁-C₇-alkoxy, C₁-C₇-alkanoyloxy-C₁-C₇-alkyl, C₁-C₇-alkoxy-C₁-C₇-alkylamino-C₁-C₇-alkyl, C₁-C₇-alkanoylamino-C₁-C₇-alkyl, C₁-C₇-alkanoylamino, N—C₁-C₇-alkoxy-C₁-C₇-alkyl-amino, N—C₁-C₇-alkanoyl-N—C₁-C₇-alkoxy-C₁-C₇-alkyl-amino, C₁-C₇-alkylsulfonylamino-C₁-C₇-alkyl, carboxy-C₁-C₇-alkyl, C₁-C₇-alkoxycarbonyl-C₁-C₇-alkyl, C₁-C₇-alkoxy-C₁-C₇-alkoxy, amino-C₁-C₇-alkoxy, N—C₁-C₇-alkanoylamino-C₁-C₇-alkoxy, carbamoyl-C₁-C₇-alkyl, N—C₁-C₇-alkylcarbamoyl-C₁-C₇-alkyl, N—C₁-C₇-haloalkylcarbamoyl-C₁-C₇-alkyl, carbamoyl-C₁-C₇-alkoxy, N—C₁-C₇-alkylcarbamoyl-C₁-C₇-alkoxy, C₁-C₇-alkanoyl, C₁-C₇-alkyloxy-C₁-C₇-alkanoyl, C₁-C₇-alkoxy-C₁-C₇-alkanoyl, carbamoyl and N—C₁-C₇-alkoxy-C₁-C₇-alkylcarbamoyl, more preferably C₁-C₇-alkyl, —O—C₁-C₇-alkyl, halo-C₁-C₇-alkyl, halo, cyano, hydroxy-C₁-C₇-alkyl, C₁-C₇-alkoxy-C₁-C₇-alkoxy, C₁-C₇-alkanoylamino-C₁-C₇-alkyl, C₁-C₇-alkanoylamino, N—C₁-C₇-alkoxy-C₁-C₇-alkyl-amino, N—C₁-C₇-alkanoyl-N—C₁-C₇-alkoxy-C₁-C₇-alkyl-amino, in particular, methyl, O-methyl, Cl, Br, CN, methoxypropyloxy, N(methoxypropyl)-amino, N(acetyl)-amino, and N(methoxypropyl)(acetyl)-amino.

In a third embodiment R2 is preferably unsubstituted or substituted heterocyclyl.

The heterocyclyl moietyl preferably mono- or bicyclic, more preferably bicyclic. Preferred are aromatic ring systems, or partially saturated ring systems, in particular whereby one of the rings is aromatic and the other is saturated or partially saturated, most preferred are partially saturated. The heterocyclyl moiety has preferably 1, 2 or 3, more preferably 1 or 2, most preferably 2, heteroatoms selected from O, N or S, more preferably O or N. The ring system contains preferably an oxo moiety. Particularly preferred examples include bicyclic 10-membered rings preferably containing a nitrogen atom, in particular, quinolyl, isoquinolyl, 1,2,3,4-tetrahydro-1,4-benzoxazinyl, 2H-1,4-benzoxazin-3(4H)-only, 3,4-dihydro-1H-quinolin-2-onyl, or 4H-benzo[1,4]thiazin-3-onyl; or bicyclic 9-membered ring systems preferably containing a N atom, in particular indolyl, 1H-indazolyl, benzothiophenyl, imidazo[1,2-a]pyridyl or 3H-benzooxazol-2-onyl, where each heterocyclyl is unsubstituted or substituted by one or more, e.g. up to three, substituents independently selected from the group consisting of C₁-C₇-alkyl, hydroxy-C₁-C₇-alkyl, C₁-C₇-alkoxy-C₁-C₇-alkyl, C₁-C₇-alkoxy-C₁-C₇-alkoxy-C₁-C₇-alkyl, C₁-C₇-alkanoyloxy-C₁-C₇-alkyl, amino-C₁-C₇-alkyl, C₁-C₇-alkoxy-C₁-C₇-alkylamino-C₁-C₇-alkyl, C₁-C₇-alkanoylamino-C₁-C₇-alkyl, C₁-C₇-alkylsulfonylamino-C₁-C₇-alkyl, carboxy-C₁-C₇-alkyl, C₁-C₇-alkoxycarbonyl-C₁-C₇-alkyl, halo, hydroxy, C₁-C₇-alkoxy, C₁-C₇-alkoxy-C₁-C₇-alkoxy, carboxy-C₁-C₇-alkoxy, amino-C₁-C₇-alkoxy, N—C₁-C₇-alkanoylamino-C₁-C₇-alkoxy, carbamoyl-C₁-C₇-alkyl, carbamoyl-C₁-C₇-alkoxy, N—C₁-C₇-alkylcarbamoyl-C₁-C₇-alkoxy, C₁-C₇-alkanoyl, C₁-C₇-alkyloxy-C₁-C₇-alkanoyl, C₁-C₇-alkoxy-C₁-C₇-alkanoyl, carboxyl, carbamoyl and N—C₁-C₇-alkoxy-C₁-C₇-alkylcarbamoyl, more preferably C₁-C₇-alkyl, halo, hydroxy-C₁-C₇-alkyl, C₁-C₇-alkoxy-C₁-C₇-alkyl, C₁-C₇-alkanoylamino-C₁-C₇-alkyl, C₁-C₇-alkoxy-C₁-C₇-alkoxy, carbamoyl-C₁-C₇-alkyl, N—C₁-C₇-alkylcarbamoyl-C₁-C₇-alkyl, N—C₁-C₇-haloalkylcarbamoyl-C₁-C₇-alkyl, in particular methyl, pentyl, methoxy-propyl, methoxy-butyl, ethoxy-ethyl, hydroxy-butyl, methoxypropyloxy, F, CH₃—C(O)—NH—CH₂CH₂, NH₂—CO—CH₂CH₂CH₂, N(CH₂CH₃)—CO—CH₂, N(CH₂CF₃)—CO—CH₂. The heterocyclyl moiety is preferably substituted on the N if present.

Thus preferably R2 is phenyl-C₁-C₇-alkyl, di-(phenyl)-C₁-C₇-alkyl, naphthyl-C₁-C₇-alkyl, phenyl, naphthyl, pyridyl-C₁-C₇-alkyl, indolyl-C₁-C₇-alkyl, 1H-indazolyl-C₁-C₇-alkyl, quinolyl-C₁-C₇-alkyl, isoquinolyl-C₁-C₇-alkyl, 1,2,3,4-tetrahydro-1,4-benzoxazinyl-C₁-C₇-alkyl, 2H-1,4-benzoxazin-3(4H)-onyl-C₁-C₇-alkyl, 9-xanthenyl-C₁-C₇-alkyl, 1-benzothiophenyl-C₁-C₇-alkyl, pyridyl, indolyl, 1H-indazolyl, quinolyl, isoquinolyl, 1,2,3,4-tetrahydro-1,4-benzoxazinyl, 2H-1,4-benzoxazin-3(4H)-onyl, 9-xanthenyl or 1-benzothiophenyl, 3,4-Dihydro-1H-quinolin-2-onyl, 4H-Benzo[1,4]thiazin-3-onyl, 3H-benzooxazol-2-onyl, where each phenyl, naphthyl, pyridyl, indolyl, 1H-indazolyl, quinolyl, isoquinolyl, 1,2,3,4-tetrahydro-1,4-benzoxazinyl, 2H-1,4-benzoxazin-3(4H)-onyl or 1-benzothiophenyl is unsubstituted or substituted by one or more, e.g. up to three, substituents independently selected from the group consisting of C₁-C₇-alkyl, hydroxy-C₁-C₇-alkyl, C₁-C₇-alkoxy-C₁-C₇-alkyl, C₁-C₇-alkoxy-C₁-C₇-alkoxy-C₁-C₇-alkyl, C₁-C₇-alkanoyloxy-C₁-C₇-alkyl, amino-C₁-C₇-alkyl, C₁-C₇-alkoxy-C₁-C₇-alkylamino-C₁-C₇-alkyl, C₁-C₇-alkanoylamino-C₁-C₇-alkyl, C₁-C₇-alkylsulfonylamino-C₁-C₇-alkyl, carboxy-C₁-C₇-alkyl, C₁-C₇-alkoxycarbonyl-C₁-C₇-alkyl, halo, hydroxy, C₁-C₇-alkoxy, C₁-C₇-alkoxy-C₁-C₇-alkoxy, amino-C₁-C₇-alkoxy, N—C₁-C₇-alkanoylamino-C₁-C₇-alkoxy, carbamoyl-C₁-C₇-alkoxy, N—C₁-C₇-alkylcarbamoyl-C₁-C₇-alkoxy, C₁-C₇-alkanoyl, C₁-C₇-alkyloxy-C₁-C₇-alkanoyl, C₁-C₇-alkoxy-C₁-C₇-alkanoyl, carboxyl, carbamoyl and N—C₁-C₇-alkoxy-C₁-C₇-alkylcarbamoyl; more preferably R2 is phenyl-C₁-C₇-alkyl, di-(phenyl)-C₁-C₇-alkyl, phenyl, indolyl-C₁-C₇-alkyl, 1H-indazolyl-C₁-C₇-alkyl, 9-xanthenyl-C₁-C₇-alkyl, 1,2,3,4-tetrahydro-1,4-benzoxazinyl or 2H-1,4-benzoxazin-3(4H)-onyl, where each phenyl, indolyl, 1H-indazolyl, 1,2,3,4-tetrahydro-1,4-benzoxazinyl, 2H-1,4-benzoxazin-3(4H)-only or 9-xanthenyl is unsubstituted or substituted by up to three substituents independently selected from the group consisting of C₁-C₇-alkyl, C₁-C₇-alkoxy-C₁-C₇-alkyl, C₁-C₇-alkoxy-C₁-C₇-alkoxy-C₁-C₇-alkyl, C₁-C₇-alkanoylamino-C₁-C₇-alkyl, C₁-C₇-alkylsulfonylamino-C₁-C₇-alkyl, carboxy-C₁-C₇-alkyl, C₁-C₇-alkoxycarbonyl-C₁-C₇-alkyl, halo, C₁-C₇-alkoxy, C₁-C₇-alkoxy-C₁-C₇-alkoxy and C₁-C₇-alkyloxy-C₁-C₇-alkanoyl.

Particularly preferred examples for R2 are

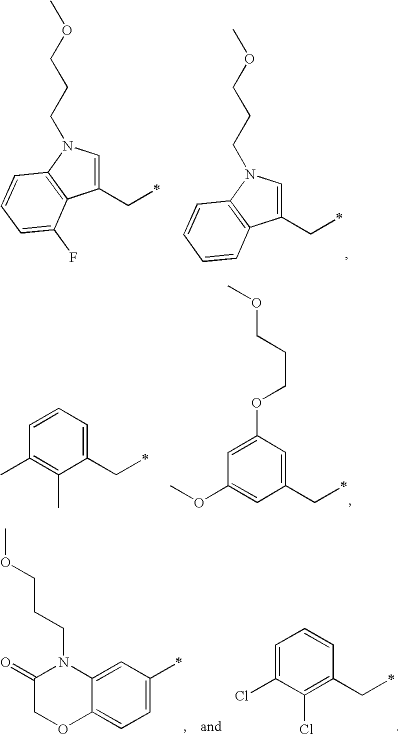

Preferred Definitions for W

As W, a moiety of the formula IA or IC is preferred.

In a moiety W of the formula IA, preferably one of X₁ and X₂ is nitrogen or CH, while the other and X₃, X₄ and X₅ are CH.

In a moiety W if the formula IC, preferably X₁ is CH₂, NH, S or O and one of X₂, X₃ and X₄ is N, while the others are CH, with the proviso that at least one ring nitrogen (N or in the case or X₁ NH) is present. R3 is then preferably bound to X₂ or more preferably to X₄ or especially to X₃ instead of a hydrogen.

The skilled person will understand that a substituent R3 (and, where present, R4) can only be present at the position of and instead of a hydrogen bound to a ring member X₁ to X₄ selected from CH, CH₂ or NH so that only four-bonded carbon or three-bonded nitrogen (which, in the case of salt formation, may however be protonated to become four-bonded and then positively charged) is present.

Most preferably, W is a moiety of formula (IA) such as phenyl or pyridyl, preferably phenyl. In another embodiment W is a moiety of formula (IC) such as oxazole.

Preferred Definitions for y and z

y is preferably 0 or 1, most preferably 0, and z is 0 or 1, most preferably 0.

In one preferred embodiment of the invention, R3 is bound to X₃ or to X₄ in formula IA, formula IB or formula IC. In another preferred embodiment, R3 is bound to X₁ in formula IA or formula IB or to X₂ in formula IA, formula IB or formula IC.

Preferred Definitions for R3

As R3, phenyl, pyridyl, hydroxyphenyl, mono- or di-(C₁-C₇-alkyloxy)-phenyl-C₁-C₇-alkyloxy, are especially preferred.

Most preferably, these moieties are bound to X₃ or to X₄ or in the case of formula IA and IB to X₁ or X₂, in the case of formula IC to X₂. More generally, R₃ is hydrogen or more preferably a moiety different from hydrogen selected from the definitions for R₃ herein.

In a first embodiment, R3 is preferably substituted or unsubstituted aryl.

Preferred examples of aryl include phenyl or naphthyl, more preferably phenyl. In one embodiment, R3 is preferably unsubstituted phenyl. In another embodiment, R3 is substituted phenyl. When the aryl moiety is substituted, it is preferably mono- di- or tri-substituted, more preferably mono- or di-substituted. Most preferably aryl is mono-substituted. Suitable substituents are as defined herein, preferably C₁-C₇-alkyl, hydroxy, C₁-C₇-alkoxy, halo-C₁-C₇-alkyl, carboxy-C₁-C₇-alkyl, carboxy-C₁-C₇-alkenyl, halo-C₁-C₇-alkyloxy, phenyl-C₁-C₇-alkoxy wherein phenyl is unsubstituted or substituted by C₁-C₇-alkoxy and/or halo, carboxy-C₁-C₇-alkyloxy, C₁-C₇-alkyloxy-carbonyl-C₁-C₇-alkyloxy, hydroxy-C₁-C₇-alkyloxy, amino-C₁-C₇-alkyloxy, carboxy-hydroxy-C₁-C₇-alkyloxy, aminocarbonyl-C₁-C₇-alkyloxy, N-mono- or N,N-di-(C₁-C₇-alkyl)-aminocarbonyl-C₁-C₇-alkyloxy, N-mono- or N,N-di-(hydroxyl-C₁-C₇-alkyl)-aminocarbonyl-C₁-C₇-alkyloxy, alkylsulfonylamino-C₁-C₇-alkyloxy, alkylsulfonylaminocarbonyl-C₁-C₇-alkyloxy, halo, amino. More preferably the substituent is selected from halo such as F, hydroxyl, cyano, C₁-C₇-alkyloxy such as methoxy.

In a second embodiment, R3 is preferably substituted or unsubstituted heterocyclyl.

The heterocyclyl moiety is preferably mono- or bicyclic, more preferably monocyclic. Preferred are aromatic ring systems, saturated or partially saturated ring systems, in particular whereby one of the rings is aromatic and the other is saturated or partially saturated, most preferred are partially saturated. The heterocyclyl moiety has preferably 1, 2 or 3, more preferably 1 or 2, most preferably 1, heteroatom selected from O, N or S, more preferably O or N. The ring system contains preferably an oxo moiety. Particularly preferred examples include monocyclic 4, 5 or 6-membered rings preferably containing a nitrogen atom, in particular, pyridyl, thiophenyl, pyrazolyl, pyridazinyl, piperidyl, azetidinyl, tetrazolyl, triazolyl, 1,2,3,6-tetrahydropyridyl, and pyrrolyl, specifically pyridyl. Each heterocyclyl is unsubstituted or substituted by one or more, e.g. up to three, preferably 1, substituents independently selected from the group consisting of C₁-C₇-alkyl, hydroxy-C₁-C₇-alkyl, C₁-C₇-alkoxy-C₁-C₇-alkyl, C₁-C₇-alkanoyloxy-C₁-C₇-alkyl, amino-C₁-C₇-alkyl, C₁-C₇-alkoxy, C₁-C₇-alkanoylamino-C₁-C₇-alkyl, carboxy-C₁-C₇-alkyl, C₁-C₇-alkoxycarbonyl-C₁-C₇-alkyl, halo, hydroxy, C₁-C₇-alkoxy-C₁-C₇-alkoxy, carboxy-C₁-C₇-alkoxy, amino-C₁-C₇-alkoxy, N—C₁-C₇-alkanoylamino-C₁-C₇-alkoxy, carbamoyl-C₁-C₇-alkyl, carbamoyl-C₁-C₇-alkoxy, C₁-C₇-alkanoyl, C₁-C₇-alkyloxy-C₁-C₇-alkanoyl, C₁-C₇-alkoxy-C₁-C₇-alkanoyl, carboxyl, carbamoyl, N-mono- or N,N-di-(C₁-C₇-alkyl)amino and amino, more preferably of C₁-C₇-alkyl, amino-C₁-C₇-alkyl, C₁-C₇-alkoxy, carboxy-C₁-C₇-alkyl, carboxy-C₁-C₇-alkoxy, carboxyl, N-mono- or N,N-di-(C₁-C₇-alkyl)amino and amino, in particular methyl, amino, dimethylamino, carboxy, carboxymethyl, aminomethyl, methoxy, and carboxymethoxy. Preferably heterocyclyl is unsubstituted.

When R3 is heterocyclyl, R2 is preferably substituted alkyl as explained above, in particular

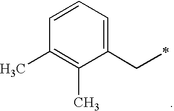

In a third embodiment R3 is preferably hydroxyl or C₁-C₇-alkoxy, preferably ethoxy or methoxy, most preferably methoxy.

In this embodiment R4 is preferably absent. In this embodiment R2 is preferably substituted alkyl as explained above, in particular

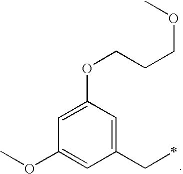

In a fourth embodiment R3 is preferably etherified hydroxyl.

Etherified hydroxyl is as defined herein, preferably the H of the OH group has been replaced by a substituted or unsubstituted alkyl. Preferred examples for alkyl are branched or straight chain C₁-C₇-alkyl which may be substituted or unsubstituted. Preferred examples include methyl, ethyl, isopropyl, n-propyl, n-butyl, sec-butyl or tert-butyl, more preferably methyl, ethyl or isopropyl, most preferably methyl. In one embodiment the alkyl moiety is preferably substituted such as mono-substituted. Examples of suitable substituents are as defined herein, preferably C₁-C₇-alkyloxy such as methoxy; aryl, such as phenyl, which may be substituted by one to three, preferably two substituents as defined herein, e.g. C₁-C₇-alkyloxy such as methoxy; and heterocyclyl such as mono- or bicyclic, more preferably monocyclic, preferably aromatic or saturated ring systems, having preferably 1, 2 or 3, more preferably 1, heteroatom selected from O, N or S, more preferably O or N, in particular 5-or 6-membered ringssuch as pyridyl or tetrahydrofuranyl, which may be substituted by one to three, preferably one substituent as defined herein, e.g. amino, -mono- or N,N-di-(C₁-C₇-alkyl)amino. The most preferred substituent is aryl as defined above.

In this embodiment R4 is preferably absent. In this embodiment R2 is preferably substituted alkyl as explained above, in particular

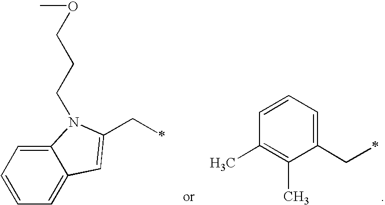

Particularly preferred examples for W-R3 are

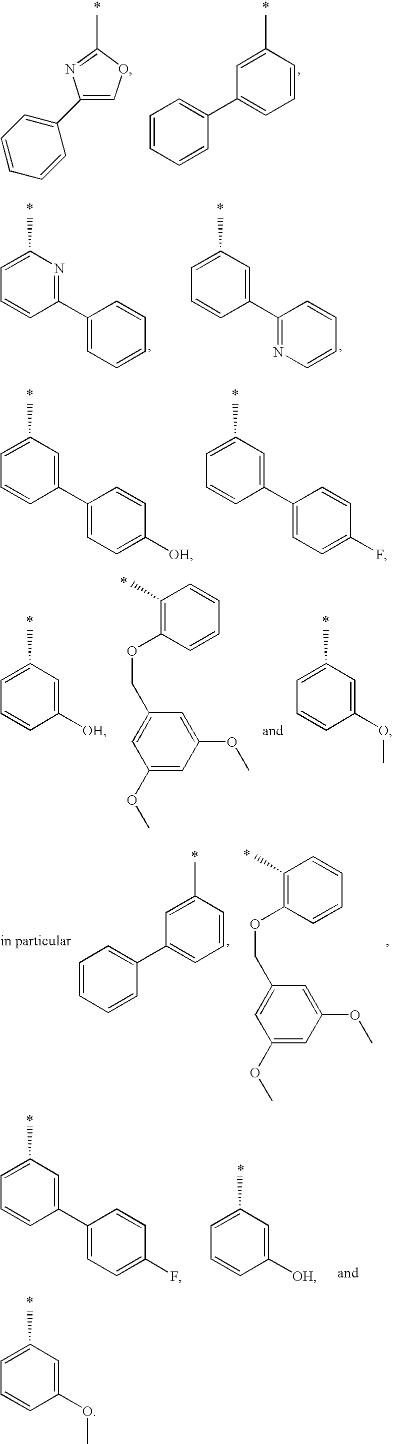

More preferred is

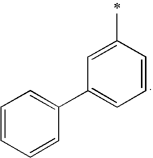

Preferred Definitions for R4

As R₄, hydroxy, halo, such as F, or C₁-C₇-alkoxy are especially preferred or R₄ is absent. In another embodiment R4 is C₁-C₇-alkyl, such as methyl. Most preferably, R4 is absent.

Preferred Definitions for T

T is preferably either methylene (—CH₂—) or carbonyl (—C(═O)—), more preferably carbonyl.

In all definitions above and below the person having skill in the art will, without undue experimentation or considerations, be able to recognize which are relevant (e.g. those that are sufficiently stable for the manufacture of pharmaceuticals, e.g. having a half-life of more than 30 seconds) and thus are preferably encompassed by the present claims and that only chemically feasible bonds and substitutions (e.g. in the case of double or triple bonds, hydrogen carrying amino or hydroxy groups and the like) are encompassed, as well as tautomeric forms where present. For example, preferably, for reasons of stability or chemical feasability, in -G-R5 G and the atom binding as part of R5 are not simultaneously oxy plus oxy, thio plus oxy, oxy plus thio or thio plus thio. Substitutents binding via an O or S that is part of them are preferably not bound to nitrogen e.g. in rings.

Salts are especially the pharmaceutically acceptable salts of compounds of formula I. They can be formed where salt forming groups, such as basic or acidic groups, are present that can exist in dissociated form at least partially, e.g. in a pH range from 4 to 10 in aqueous solutions, or can be isolated especially in solid, especially crystalline, form.

Such salts are formed, for example, as acid addition salts, preferably with organic or inorganic acids, from compounds of formula I with a basic nitrogen atom (e.g. imino or amino), especially the pharmaceutically acceptable salts. Suitable inorganic acids are, for example, halogen acids, such as hydrochloric acid, sulfuric acid, or phosphoric acid. Suitable organic acids are, for example, carboxylic, phosphonic, sulfonic or sulfamic acids, for example acetic acid, propionic acid, lactic acid, fumaric acid, succinic acid, citric acid, amino acids, such as glutamic acid or aspartic acid, maleic acid, hydroxymaleic acid, methylmaleic acid, benzoic acid, methane- or ethane-sulfonic acid, ethane-1,2-disulfonic acid, benzenesulfonic acid, 2-naphthalenesulfonic acid, 1,5-naphthalene-disulfonic acid, N-cyclohexylsulfamic acid, N-methyl-, N-ethyl- or N-propyl-sulfamic acid, or other organic protonic acids, such as ascorbic acid.

In the presence of negatively charged radicals, such as carboxy or sulfo, salts may also be formed with bases, e.g. metal or ammonium salts, such as alkali metal or alkaline earth metal salts, for example sodium, potassium, magnesium or calcium salts, or ammonium salts with ammonia or suitable organic amines, such as tertiary monoamines, for example triethylamine or tri(2-hydroxyethyl)amine, or heterocyclic bases, for example N-ethyl-piperidine or N,N′-dimethylpiperazine.

When a basic group and an acid group are present in the same molecule, a compound of formula I may also form internal salts.

For isolation or purification purposes it is also possible to use pharmaceutically unacceptable salts, for example picrates or perchlorates. For therapeutic use, only pharmaceutically acceptable salts or free compounds are employed (where applicable comprised in pharmaceutical preparations), and these are therefore preferred.

In view of the close relationship between the compounds in free form and in the form of their salts, including those salts that can be used as intermediates, for example in the purification or identification of the compounds or salts thereof, any reference to “compounds”, “starting materials” and “intermediates” hereinbefore and hereinafter, especially to the compound(s) of the formula I, is to be understood as referring also to one or more salts thereof or a mixture of a corresponding free compound and one or more salts thereof, each of which is intended to include also any solvate, metabolic precursor such as ester or amide of the compound of formula I, or salt of any one or more of these, as appropriate and expedient and if not explicitly mentioned otherwise. Different crystal forms may be obtainable and then are also included.

Where the plural form is used for compounds, starting materials, intermediates, salts, pharmaceutical preparations, diseases, disorders and the like, this is intended to mean one (preferred) or more single compound(s), salt(s), pharmaceutical preparation(s), disease(s), disorder(s) or the like, where the singular or the indefinite article (“a”, “an”) is used, this is intended to include the plural or preferably the singular.

The compounds of the present invention possess two or more asymmetric centers depending on the choice of the substituents. The preferred absolute configurations are as indicated herein specifically. However, any possible isolated or pure diastereoisomers, enantiomers or geometric enantiomers, and mixtures thereof, e.g., mixtures of enantiomers, such as racemates, are encompassed by the present invention.

As described herein above, the present invention provides 3,4,5-substituted piperidine derivatives of formula I, these compounds for use in the (prophylactic and/or therapeutic) treatment of a disease (=condition, disorder) in a warm-blooded animal, especially a human, preferably of a disease dependent on (especially inappropriate) renin activity, a pharmaceutical composition comprising a compound of the formula I, methods for preparing said compound or pharmaceutical preparation, and methods of treating conditions dependent on (especially inappropriate) renin activity by administration of a therapeutically effective amount of a compound of the formula I, or a pharmaceutical composition thereof.

“Inappropriate” renin activity preferably relates to a state of a warm-blooded animal, especially a human, where renin shows a renin activity that is too high in the given situation (e.g. due to one or more of misregulation, overexpression e.g. due to gene amplification or chromosome rearrangement or infection by microorganisms such as virus that express an aberrant gene, abnormal activity e.g. leading to an erroneous substrate specificity or a hyperactive renin e.g. produced in normal amounts, too low activity of renin activity product removing pathways, high substrate concentration and/or the like) and/or leads to or supports a renin dependent disease or disorder as mentioned above and below, e.g. by too high renin activity. Such inappropriate renin activity may, for example, comprise a higher than normal activity, or further an activity in the normal or even below the normal range which, however, due to preceding, parallel and or subsequent processes, e.g. signaling, regulatory effect on other processes, higher substrate or product concentration and the like, leads to direct or indirect support or maintenance of a disease or disorder, and/or an activity that supports the outbreak and/or presence of a disease or disorder in any other way. The inappropriate activity of renin may or may not be dependent on parallel other mechanisms supporting the disorder or disease, and/or the prophylactic or therapeutic effect may or may include other mechanisms in addition to inhibition of renin. Therefore “dependent” has to be read as “dependent inter alia”, (especially in cases where a disease or disorder is really exclusively dependent only on renin) preferably as “dependent mainly”, more preferably as “dependent essentially only”. A disease dependent on (especially inappropriate) activity of renin may also be one that simply responds to modulation of renin activity, especially responding in a beneficial way (e.g. lowering the blood pressure) in case of renin inhibition.

Where a disease or disorder dependent on inappropriate activity of a renin is mentioned (such in the definition of “use” in the following paragraph and also especially where a compound of the formula I is mentioned for use in the diagnostic or therapeutic treatment which is preferably the treatment of a disease or disorder dependent on inappropriate renin activity, this refers preferably to any one or more diseases or disorders that depend on inappropriate activity of natural renin and/or one or more altered or mutated forms thereof.

Where subsequently or above the term “use” is mentioned (as verb or noun) (relating to the use of a compound of the formula I or of a pharmaceutically acceptable salt thereof, or a method of use thereof), this (if not indicated differently or to be read differently in the context) includes any one or more of the following embodiments of the invention, respectively (if not stated otherwise): the use in the treatment of a disease or disorder that depends on (especially inappropriate) activity of renin, the use for the manufacture of pharmaceutical compositions for use in the treatment of a disease or disorder that depends on (especially inappropriate) activity of renin; a method of use of one or more compounds of the formula I in the treatment of a disease or disorder that depends on (especially inappropriate) activity of renin; a pharmaceutical preparation comprising one or more compounds of the formula I for the treatment of a disease or disorder that depends on (especially inappropriate) activity of renin; and one or more compounds of the formula I for use in the treatment of a disease or disorder in a warm-blooded animal, especially a human, preferably a disease that depends on (especially inappropriate) activity of renin; as appropriate and expedient, if not stated otherwise.

The terms “treat”, “treatment” or “therapy” refer to the prophylactic (e.g. delaying or preventing the onset of a disease or disorder) or preferably therapeutic (including but not limited to preventive, delay of onset and/or progression, palliative, curing, symptom-alleviating, symptom-reducing, patient condition ameliorating, renin-modulating and/or renin-inhibiting) treatment of said disease(s) or disorder(s), especially of the one or more disease or disorder mentioned above or below.

### PREFERRED EMBODIMENTS ACCORDING TO THE INVENTION

The groups of preferred embodiments of the invention mentioned below are not to be regarded as exclusive, rather, e.g., in order to replace general expressions or symbols with more specific definitions, parts of those groups of compounds can be interchanged or exchanged using the definitions given above, or omitted, as appropriate, and each of the more specific definitions, independent of any others, may be introduced independently of or together with one or more other more specific definitions for other more general expressions or symbols.

Preferred is a compound of the formula I with a configuration given in the following formula A:

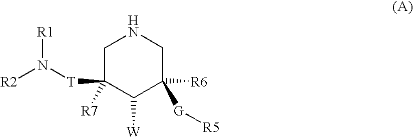

wherein R1, R2, R5, R6, R7, T, W and G are as defined above or below, with the proviso that G-R5 and R6 are not together oxo, or a (preferably pharmaceutically acceptable) salt thereof, especially in essentially pure (this preferably meaning wherever mentioned herein in 80% or more purity, more preferably in 95% or more purity with regard to the presence of other isomers, especially the mirror image (=antipode)) form. The enantiomer with the configuration of the mirror image (the mirror image form or antipode) of the compound of formula A, i.e. a compound of the formula C,

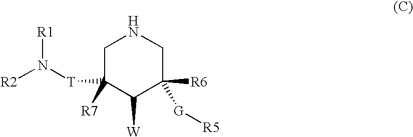

wherein the substituents are as defined under formula A, is somewhat less preferred.

Preferred is also a compound of the formula I a configuration given in the following formula B:

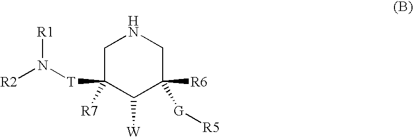

wherein R1, R2, R5, R6, R7, T, W and G are as defined above or below, with the proviso that G-R5 and R6 are not together oxo, or a (preferably pharmaceutically acceptable) salt thereof, especially in essentially pure (this preferably meaning wherever mentioned herein in 80% or more purity, more preferably in 95% or more purity with regard to the presence of other isomers, especially the mirror image) form. The corresponding enantiomer with the configuration of the mirror image of the compound of formula B is somewhat less preferred.

Preferred is a compound of the formula I, wherein

R1 is hydrogen, C₃-C₈-cycloalkyl, C₁-C₇-alkyl or halo-lower alkyl;

R2 is phenyl-C₁-C₇-alkyl wherein phenyl is unsubstituted or substituted by one or more, preferably up to three, moieties independently selected from the group consisting of C₁-C₇-alkyl, halo, C₁-C₇-alkyloxy-C₁-C₇-alkyloxy and C₁-C₇-alkyloxy; indolyl, or with slightly less preference benzoxazinonyl, indolyl- or benzoxazinonyl-C₁-C₇-alkyl, wherein where mentioned hereinbefore indolyl and benzoxazinyl is unsubstituted or substituted by one or more, preferably up to three, substituents independently selected from halo, C₁-C₇-alkyloxy and C₁-C₇-alkoxy-C₁-C₇-alkyl;

W is a moiety of the formula IA wherein one of X₁ and X₂ is nitrogen or CH, while the other and X₃, X₄ and X₅ are CH; or a moiety of the formula IC shown in claim 1, wherein X₁ is CH₂, NH, S or O and one of X₂, X₃ and X₄ is N, while the others are CH, with the proviso that at least one ring nitrogen N or in the case or X₁ NH is present;

z and y are 0;

R3 is phenyl or phenyl-C₁-C₇-alkoxy, where in both cases phenyl is unsubstituted or substituted by one or more, preferably up to three, moieties independently selected from hydroxy and C₁-C₇-alkyloxy; with the proviso that R3 in the case where W is a moiety of the formula IA is bound either to X₃ or to X₄ or to X₁ or to X₂, in the case where W is a moiety of the formula IA is bound either to X₃ or X₄, or to X₂; or (with slightly less preference) is pyridyl, e.g. 2-pyridyl;

G is methylene, oxy, thio, imino or substituted imino —NR8- wherein R8 is C₁-C₇-alkyl or unsubstituted or halo-substituted phenyl-C₁-C₇-alkyl;

R5 is hydrogen, C₁-C₇-alkyl that is unsubstituted or substituted by one or more, preferably up to three, moieties independently selected from phenyl, C₁-C₇-alkoxy and hydroxy; or, if G is methylene, can have one of the meanings just mentioned or alternatively be selected from phenyl, C₁-C₇-alkoxy-C₁-C₇-alkyl, C₁-C₇-alkoxy-C₁-C₇-alkoxy-C₁-C₇-alkyl, C₁-C₇-alkoxy and C₁-C₇-alkoxy-C₁-C₇-alkoxy; or, if G is oxy, thio, imino or —NR8-, can be selected from hydrogen, C₁-C₇-alkyl that is unsubstituted or substituted by one or more, preferably up to three, moieties independently selected from phenyl, C₁-C₇-alkoxy and hydroxy, C₁-C₇-alkanoyl, such as acetyl, 3,3-dimethyl-butyryl, 2,2-dimethyl-propionyl or 3,3-dimethyl-butyryl, unsubstituted or mono-, di- or tri-(halo and/or C₁-C₇-alkyl)-substituted benzoyl or naphthoyl, such as 4-methyl-benzoyl, C₃-C₈-cycloalkylcarbonyl, such as cyclobutylcarbonyl, unsubstituted or phenyl-substituted pyrrolidinylcarbonyl, especially phenyl-pyrrolidinocarbonyl, C₁-C₇-alkylsulfonyl, such as methylsulfonyl (=methanesulfonyl), (phenyl- or naphthyl)-C₁-C₇-alkylsulfonyl, such as phenylmethanesulfonyl, or (unsubstituted, or [C₁-C₇-alkyl-, phenyl-, halo-lower alkyl-, halo, oxo-C₁-C₇-alkyl-C₁-C₇-alkyloxy-, phenyl-C₁-C₇-alkoxy-, halo-C₁-C₇-alkyloxy-, phenoxy-, C₁-C₇-alkanoylamino-, cyano-, C₁-C₇-alkanoyl- and/or C₁-C₇-alkylsulfonyl-]substituted) (phenyl-or naphthyl)-sulfonyl, such as phenylsulfonyl, naphthalene-1-sulfonyl, naphthalene-2-sulfonyl, toluene-4-sulfonyl, 4-isopropyl-benzenesulfonyl, biphenyl-4-sulfonyl, 2-trifluoromethyl-benzenesulfonyl, 4-chloro-benzenesulfonyl, 3-chloro-benzene-sulfonyl, 2-chloro-benzenesulfonyl, 2,4-difluoro-benzenesulfonyl, 2,6-difluoro-benzene-sulfonyl, 2,5-dichloro-benzenesulfonyl, 3,4-dichloro-benzenesulfonyl, 3,5-dichloro-benzene-sulfonyl, 2,3-dichloro-benzenesulfonyl, 3-methoxy-benzenesulfonyl, 4-methoxy-benzene-sulfonyl, 2,5-dimethoxy-benzenesulfonyl, 4-trifluoromethoxy-benzenesulfonyl, 2-benzyloxy-benzenesulfonyl, 3-trifluoromethyl-benzenesulfonyl, 4-phenoxy-benzenesulfonyl, 4-(2-oxo-propyl)-benzenesulfonyl, 4-acetylamino-benzenesulfonyl, 4-cyano-benzenesulfonyl, 2-cyano-benzenesulfonyl, 3-cyano-benzenesulfonyl, 3-acetyl-benzenesulfonyl or 4-methanesulfonyl-benzenesulfonyl, halo-thiophene-2-sulfonyl, such as 5-chloro-thiophene-2-sulfonyl, quinoline-sulfonyl, such as quinoline-8-sulfonyl, (C₁-C₇-alkanoylamino and/or C₁-C₇-alkyl)-substituted thiazol-sulfonyl, such as 2-acetylamino-4-methyl-thiazole-5-sulfonyl, (halo and/or C₁-C₇-alkyl)-substituted pyrazolesulfonyl, such as 5-chloro-1,3-dimethyl-1H-pyrazole-4-sulfonyl, pyridine-sulfonyl, such as pyridine-3-sulfonyl, or N-mono- or N,N-di-(C₁-C₇-alkyl, (unsubstituted or halo-substituted) phenyl or naphthyl, phenyl-C₁-C₇-alkyl, naphthyl-C₁-C₇-alkyl or C₃-C₈-cycloalkyl)-aminocarbonyl, such as N-tert-butyl-aminocarbonyl, (3-chloro-phenyl)-amino-carbonyl, N-benzylaminocarbonyt, N-cyclohexyl-aminocarbonyl, C₁-C₇-alkylaminocarbonyl or phenyl-C₁-C₇alkylaminocarbonyl, and (C₁-C₇-alkyl, phenyl, naphthyl, phenyl-C₁-C₇-alkyl and/or napthyl-C₁-C₇-alkyl)-oxycarbonyl, e.g. C₁-C₇-alkoxycarbonyl, such as tert-butyloxy-carbonyl or isobutyloxycarbonyl, or phenyl-C₁-C₇-alkyloxycarbonyl; especially from C₁-C₇-alkanoyl, C₁-C₇-alkylsulfonyl, phenylsulfonyl and C₁-C₇-alkoxycarbonyl. or G-R5 is halo, especially fluoro;

R6 is hydrogen, C₁-C₇-alkyl or halo, especially fluoro;

or G-R5 and R6 together are oxo as such and/or in hydrated form as two hydroxy groups;

R7 is hydrogen; and

T is carbonyl;

or a pharmaceutically acceptable salt thereof.

Particular embodiments of the invention, especially of compounds of the formula I and/or salts thereof, are provided in the Examples—the invention thus, in a very preferred embodiment, relates to a compound of the formula I, or a salt thereof, selected from the compounds given in the Examples, as well as the use thereof.

Process of Manufacture

A compound of formula I, or a salt thereof, is prepared analogously to methods that, for other compounds, are in principle known in the art, so that for the novel compounds of the formula I the process is novel at least as analogy process, especially as described or in analogy to methods described herein in the illustrative Examples, or modifications thereof, preferably in general by

(a) for the synthesis of a compound of the formula I wherein the moieties are as defined for a compound of the formula I, reacting a carbonic acid compound of the formula II

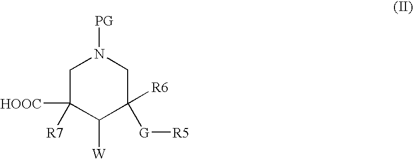

wherein W, G, R5, R6 and R7-G-R5 are as defined for a compound of the formula I and PG is a protecting group, or an active derivative thereof, with an amine of the formula III,

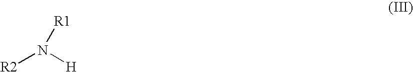

wherein R1 and R2 are as defined for a compound of the formula I, and removing protecting groups to give the corresponding compound of the formula I, or

(b) for the preparation of a compound of the formula I wherein R₃ is unsubstituted or substituted aryl or unsubstituted or substituted alkyloxy and W is a moiety of the formula IA given above, by reacting a compound of the formula IV,

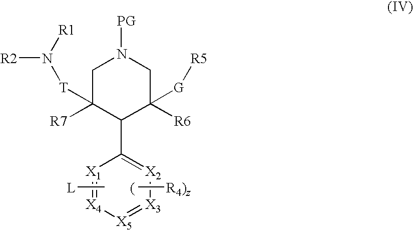

wherein R1, R2, T, G, R5, R6, R7, X₁, X₂, X₃, X₄, X₅, z and R4 are as defined for a compound of the formula I, PG is a protecting group and L is a leaving group or hydroxy, with a compound of the formula V,

R3-Q (V)

wherein R3 is as just defined and Q is —B(OH)₂ or a leaving group, and removing protecting groups to give the corresponding compound of the formula I,

and, if desired, subsequent to any one or more of the processes mentioned above converting an obtainable compound of the formula I or a protected form thereof into a different compound of the formula I, converting a salt of an obtainable compound of formula I into the free compound or a different salt, converting an obtainable free compound of formula I into a salt thereof, and/or separating an obtainable mixture of isomers of a compound of formula I into individual isomers;

where in any of the starting materials (especially of the formulae II to IV), in addition to specific protecting groups mentioned, further protecting groups may be present, and any protecting groups are removed at an appropriate stage in order to obtain a corresponding compound of the formula I, or a salt thereof.

Preferred Reaction Conditions

The preferred reaction conditions for the reactions mentioned above, as well as for the transformations and conversions, are as follows (or analogous to methods used in the Examples or as described there):

The reaction under (a) between an acid of the formula II, or a reactive derivative thereof, and an amino compound of the formula III preferably takes place under customary condensation conditions, where among the possible reactive derivatives of an acid of the formula II reactive esters (such as the hydroxybenzotriazole (HOBT), pentafluorophenyl, 4-nitrophenyl or N-hydroxysuccinimide ester), acid halogenides (such as the acid chloride or bromide) or reactive anhydrides (such as mixed anhydrides with lower alkanoic acids or symmetric anhydrides) are preferred. Reactive carbonic acid derivatives can also be formed in situ. The reaction is carried out by dissolving the compounds of formulae II and III in a suitable solvent, for example a halogenated hydrocarbon, such as methylene chloride, N,N-dimethylformamide, N,N-dimethylacetamide, N-methyl-2-pyrrolidone, methylene chloride, or a mixture of two or more such solvents, and by the addition of a suitable base, for example triethylamine, diisopropylethylamine (DIEA) or N-methylmorpholine and, if the reactive derivative of the acid of the formula II is formed in situ, a suitable coupling agent that forms a preferred reactive derivative of the carbonic acid of formula III in situ, for example dicyclohexylcarbodiimide/1-hydroxybenzotriazole (DCC/HOBT); bis(2-oxo-3-oxazolidinyl)phosphinic chloride (BOPCl); O-(1,2-dihydro-2-oxo-1-pyridyl)-N,N,N′,N′-tetramethyluronium tetrafluoroborate (TPTU); O-benzotriazol-1-yl)-N,N,N′, N′-tetramethyluronium tetrafluoroborate (TBTU); (benzotriazol-1-yloxy)-tripyrrolidinophosphonium-hexafluorophosphate (PyBOP), 1-(3-dimethylaminopropyl)-3-ethylcarbodiimide hydrochloride/hydroxybenzotriazole or/1-hydroxy-7-azabenzotriazole (EDC/HOBT or EDC/HOAt) or HOAt alone, or with (1-chloro-2-methyl-propenyl)-dimethylamine. For review of some other possible coupling agents, see e.g. Klauser; Bodansky, Synthesis 1972, 453-463. The reaction mixture is preferably stirred at a temperature of between approximately −20 and 50° C., especially between 0° C. and 30° C., e.g. at room temperature. The reaction is preferably carried out under an inert gas, e.g. nitrogen or argon.

The subsequent removal of a protecting group, e.g. PG, such as tert-butoxycarbonyl, methoxymethyl, benzyl, 2-(trimethylsilyl)-ethoxycarbonyl or tert-butyldimethylsilyl, takes place under standard conditions, see also the literature mentioned below under General Process Conditions. For example, tert-butoxycarbonyl is removed in the presence of an acid, e.g. a hydrohalic acid, such as HCl, in an appropriate solvent, e.g. an ether, such as dioxane, or an alcohol, e.g. isopropanol, at customary temperatures, e.g. at room temperature, the removal of benzyl can be achieved e.g. by reaction with ethylchloroformate in an appropriate solvent, e.g. toluene, at elevated temperatures, e.g. from 80 to 110° C., and subsequent removal of the resulting ethoxycarbonyl group by hydrolysis in the presence of a base, e.g. an alkali metal hydroxide, such as potassium hydroxide, in an appropriate solvent, e.g. in an alcohol, such as ethanol, at elevated temperatures, e.g. from 80 to 120° C., or by removal by means of trimethylsilyl trifluoroacetate in a tertiary nitrogen base, such as 2,6-lutidine, in the presence of an appropriate solvent, such as a halogenated hydrocarbon, e.g. methylene chloride, the removal of 2-(trimethylsilyl)-ethoxycarbonyl can be achieved, for example, by reaction with a tetra-lower alkylammonium fluoride, such as tetraethylammoniumfluoride, in an appropriate solvent or solvent mixture, e.g. a halogenated hydrocarbon, such as methylene chloride, and/or a nitrile, such as acetonitrile, preferably at elevated temperatures, e.g. under reflux conditions, and the removal of tert-butyldimethylsilyl in the presence of tetra-butyl ammonium fluoride, e.g. in the presence of a solvent such as tetrahydrofurane at preferred temperatures from 0 to 50° C., e.g. at about room temperature. Where a hydroxy group is protected by methoxymethyl and an imino group by tert-butoxycarbonyl, these two protecting groups can be removed, e.g., by sequential treatment first with tetrabutyl-ammonium fluoride in an appropriate solvent, e.g. tetrahydrofurane, followed b y treatment with trimethylsilyl triflate in 2,6-lutidine, in both cases at preferred temperatures in the range from o to 50° C., e.g. about at room temperature.

Where the reaction under (b) takes place with a compound of the formula IV wherein L is a leaving group and with a compound of the formula V wherein Q is —B(OH)₂, L is preferably halo, such as bromo or iodo, or trifluoromethylsulfonyloxy, and the reaction preferably takes place in an appropriate solvent, such as dioxane in the presence or absence of water, a basic buffering substance, e.g. potassium phosphate or potassium carbonate, and catalyst, e.g. Pd(PPh₃)₄, at preferably elevated temperatures, e.g. between 60° C. and the reflux temperature of the mixture. Where the reaction under (b) takes place with a compound of the formula IV wherein L is hydroxy and with a compound of the formula V wherein Q is a leaving group, the leaving group is preferably halo, e.g. bromo or iodo, and the coupling reaction preferably takes place in the presence of a base, such as potassium carbonate, in an appropriate solvent, e.g. N,N-dimethylformamide, at preferably elevated temperatures, e.g. from 30 to 80° C. Removal of protecting groups can take place as described above under (a) and below in the general process conditions. Note that wherever —B(OH)₂ is mentioned, alternatively a moiety —B(OR)₂ is possible wherein the moieties OR together form a linear of branched alkylene bridge.

Where desired, R₂ other than hydrogen can subsequently be introduced by reaction with a compound of the formula VII wherein preferably D is—the reaction preferably takes place under customary substitution conditions, e.g. in the case where an aryl moiety R2 is to be coupled and Z is halo, e.g. iodo, in the presence of copper (e.g. Venus copper), sodium iodide and a base, such as potassium carbonate, in the presence or preferably absence of an appropriate solvent, e.g. at elevated temperatures in the range from, for example, 150 to 250° C., or (especially if Z in formula VIII is bromo) in the presence of a strong base, such as an alkali metal alkoholate, e.g. sodium tert-butylate, in the presence of an appropriate catalyst, such as [Pd(μ-Br)(t-Bu₃P)]₂, and of an appropriate solvent, e.g. an aromatic solvent, such as toluene, at preferred temperatures between room temperature and the reflux temperature of the mixture, or (e.g. where the moiety R2 is unsubstituted or substituted alkyl) in the presence of a base, such as an alkali metal carbonate, such as potassium carbonate, if useful in the presence of an alkali metal halogenide, e.g. sodium iodide, in an appropriate solvent, such as dimethyl formamide, at preferably elevated temperatures, e.g. between 50° C. and the reflux temperature of the mixture, or in presence of NaN(TMS)₂ in an appropriate solvent such as tetrahydrofurane at preferred temperatures from −20 to 30° C., e.g. at about 0° C., or, where R¹ is to be bound via a carbonyl or sulfonyl group, under condensation conditions e.g. as described above for reaction (a). The removal of protecting groups, both with or without preceding reaction with a compound of the formula VII, takes place e.g. as described above under the preferred conditions for reaction (a).

Optional Reactions and Conversions

Compounds of the formula I, or protected forms thereof directly obtained according to any one of the preceding procedures or after introducing protecting groups anew, which are included subsequently as starting materials for conversions as well even if not mentioned specifically, can be converted into different compounds of the formula I according to known procedures, where required after removal of protecting groups.

Where R2 is hydrogen in a compound of the formula I (or a form thereof wherein the piperidine nitrogen in protected), this can be converted into the corresponding compound wherein R2 has a meaning other than hydrogen given for compounds of the formula I by reaction with a compound of the formula IIIA,

R2*-D (IIIA)

wherein R2* is defined as R2 in a compound of the formula I and D is a leaving group, e.g. in the presence of a tertiary amine, such as triethylamine, in an appropriate solvent, such as tetrahydrofurane, at temperatures e.g. from 0 to 50° C.; or, if T is carbonyl, in the presence of a strong base, such as sodium bis(trimethylsilyl)amide in an appropriate solvent, such as tetrahydrofurane, e.g. at temperatures from −20 to 60° C., e.g. from 0 to 40° C.; or wherein D is —CHO (so that the compound of the formula IIIA is an aldehyde) and then R2* is the complementary moiety for a moiety R2 that includes a methylene group (resulting in a group R2*-CH₂—) e.g. under reaction conditions as follows: The reductive amination preferably takes place under customary conditions for reductive amination, e.g. in the presence of an appropriate hydrogenation agent, such as hydrogen in the presence of a catalyst or a complex hydride, e.g. sodium triacetoxyborohydride or sodium cyanoborohydride, in an appropriate solvent, such as a halogenated hydrocarbon, e.g. methylene chloride or 1,2,-dichloroethane, and optionally a carbonic acid, e.g. acetic acid, at preferred temperatures between −10° C. and 50° C., e.g. from 0° C. to room temperature.

Hydroxy substituents, e.g. as substitutents of aryl in alkyl substituted by aryl R1, R2 or in other aryl substituents, can be transformed into unsubstituted or substituted alkoxy, e.g. by alkylation reaction with the corresponding unsubstituted or substituted alkylhalogenide, e.g. iodide, in the presence of a base, e.g. potassium carbonate, in an appropriate solvent, e.g. N,N-dimethylformamide, e.g. at preferred temperatures between 0 and 50° C.

Where in a compound of the formula I (or a protected form thereof wherein e.g. the ring nitrogen in the piperidine ring is N-protected) G-R5 is hydroxy and R6 is hydrogen, this can be transformed by oxidation, e.g. with an appropriate oxidant, such as Dess-Martin periodinane, in a customary solvent, such as methylene chloride, for example at temperatures from 0 to 50° C., into the corresponding compound wherein G-R5 and R6 together form oxo (═O) by oxidising the C(R6)-G-R5 which is CH(OH) to the corresponding oxo compound (wherein G-R5 and R6 are oxo); after removal of any protecting group(s) present, a corresponding compound of the formula I wherein G-R5 and R6 together form oxo can be obtained.

A compound of the formula I wherein G-R5 and R6 together form oxo (or a protected form thereof e.g. protected at the nitrogen in the piperidine ring in formula I, e.g. as obtained in the last paragraph before the removal of protecting groups) can be converted into a compound of the formula I wherein G-R5 is fluoro and R6 is fluoro by reacting the oxo group with an appropriate fluorinating reagent, especially (diethylamino)sulfur trifluoride, in a customary solvent, e.g. methylene chloride, a temperatures e.g. in the range from 0 to 50° C.; after removal of any protecting group(s) present, a corresponding compound of the formula I wherein G-R5 is fluoro and R6 is fluoro can be obtained.

In a compound of the formula I (preferably in protected form e.g. at the nitrogen in the piperidine ring in formula I) wherein G-R5 is hydroxy and R6 is hydrogen, the configuration of the hydroxy G-R5 may be converted from one form or the mixture of the two possible forms to the opposite configuration by first oxidizing to the corresponding compound wherein G-R5 and R6 together form oxo (obtainable e.g. as described in the paragraph preceding the last paragraph) and then selectively oxidizing this compound with a stereoselective reagent, e.g. lithium tri-sec-butylborohydride, in a customary solvent, e.g. tetrahydrofurane, at temperatures e.g. from −100 to 25° C., e.g. at −78° C.; after removal of any protecting group(s) present, a corresponding compound of the formula I wherein G-R5 is hydroxy and R6 is hydrogen with an enriched or practically pure configuration can be obtained.

A compound of the formula I (preferably in protected form e.g. at the nitrogen in the piperidine ring in formula I) wherein G-R5 and R6 together form oxo, obtainable e.g. as described in the paragraph preceding the last two paragraphs, can be converted to a corresponding compound wherein G-R5 is hydroxy and R6 is C₁-C₇-alkyl by alkylation with a C₁-C₇-alkylmetal halogenide, e.g. a C₁-C₇-alkyl magnesium bromide, in an appropriate solvent, e.g. an ether, such as tetrahydrofurane, e.g. at temperatures from −30 to 50° C., for example at about 0° C.; after removal of any protecting group(s) present, a corresponding compound of the formula I wherein G-R5 is hydroxy and R6 is C₁-C₇-alkyl can be obtained.

A compound of the formula I (preferably in protected form e.g. at the nitrogen in the piperidine ring in formula I) wherein R5 is hydrogen and G is oxy, thio or imino may be converted to the corresponding compound wherein R5 is N-mono-(unsubstituted or substituted aryl, unsubstituted or substituted heterocyclyl, unsubstituted or substituted cycloalkyl or unsubstituted or substituted alkyl)-aminocarbonyl (bound via a —NH—C(═O)— group) and G is oxy, thio or imino by reaction with an isocyanate of the formula XX,

R5*-NCO. (XX)

wherein R5* is unsubstituted or substituted aryl, unsubstituted or substituted heterocyclyl, unsubstituted or substituted cycloalkyl or unsubstituted or substituted alkyl under customary conditions, e.g. in the presence of a strong base, such as sodium hydride, in an appropriate solvent, e.g. tetrahydrofurane, e.g. at temperatures from −30 to 30° C. After removal of any protecting group(s) present, a corresponding compound of the formula I wherein R5 is N-mono-(unsubstituted or substituted aryl, unsubstituted or substituted heterocyclyl, unsubstituted or substituted cycloalkyl or unsubstituted or substituted alkyl)-aminocarbonyl (bound via a —NH—C(═O)— group) and G is oxy, thio or imino can be obtained.

Carboxy substitutents can be converted into esterified carboxy by reaction with corresponding alcohols, e.g. C₁-C₇-alkanols, or into amidated carboxy by reaction with corresponding amines, e.g. under condensation conditions analogous to those described above under reaction (a).

Esterified carboxy substituents can be converted into free carboxy by hydrolysis, e.g. in the presence of a base, such as potassium hydroxide, in an appropriate solvent, e.g. tetrahydrofurane, preferably at elevated temperatures, e.g. from 50° C. to the reflux temperature of the reaction mixture.

A moiety -G-R5 wherein G is O and R5 is hydrogen can be converted into amino by first converting the —OH into a leaving group, e.g. by halogenation or preferably by reaction with an organic sulfonylhalogenide, such as methylsulfonylchloride, in the presence of a tertiary nitrogen base, such as triethylamine, and in the presence of an appropriate solvent, e.g. dichloromethane, preferably at lower temperatures, e.g. in the range from −30 to 20° C., followed by reaction with an alkali metal azide, e.g. sodium azide, in an appropriate solvent, such as dichloromethane, in the presence of a tertiary nitrogen base, e.g. triethylamine, and preferably at lower temperatures, e.g. in the range from −30 to 20° C. to give the corresponding azido group, which is then converted into the amino group e.g. by reaction with triphenylphosphine in an appropriate solvent, e.g. tetrahydrofurane in the presence of water, at preferably lower temperatures, e.g in the range from −30 to 20° C. Alternatively, reaction of a compound wherein G-R5 and R6 together form oxo (manufacturable as described in a preceding paragraph) with an amine R5-NR8H or R5-NH₂) under conditions of reductive amination (for example, conditions can be employed as described for the reaction of a compound of the formula IIIA; alternatively, the reaction can take place in the presence of molecular sieve, such as molecular sieve 3A, in the presence of an appropriate complex hydride, such as sodium cyanoborohydride, in an appropriate solvent, such as methanol, at low temperatures, e.g. from −90 to −50° C., using e.g. an acetate salt of the amine R5-NR8H or R5-NH₂) to give a compound of the formula wherein G-R5 is NH-R5 or NR8-R5 is possible.

A group -G-R5 wherein G is NH and R5 is H (thus being amino, obtainable e.g. according to the preceding paragraph with ammonium acetate) can be converted into the corresponding group wherein G is NH and R5 is unsubstituted or substituted alkyl or acyl by alkylation or acylation. For example, acylation may take place using the corresponding acid halogenide (e.g. the chloride) in the presence of a tertiary nitrogen base, such as triethylamine, in an appropriate solvent, such as dichloromethane, preferably at lower temperatures, e.g. in the range from −30 to 20° C. Alternatively, the acylation may take place with a compound of the formula XX as described above under analogous reaction conditions as described there.

In some cases, the conversions preferably take place with compounds of the formula I in protected form; the subsequent removal of protecting group can be achieved as above for reaction (a) and below under “General Process Conditions”, yielding a corresponding compound of the formula I.

Salts of compounds of formula I having at least one salt-forming group may be prepared in a manner known per se. For example, salts of compounds of formula I having acid groups may be formed, for example, by treating the compounds with metal compounds, such as alkali metal salts of suitable organic carboxylic acids, e.g. the sodium salt of 2-ethylhexanoic acid, with organic alkali metal or alkaline earth metal compounds, such as the corresponding hydroxides, carbonates or hydrogen carbonates, such as sodium or potassium hydroxide, carbonate or hydrogen carbonate, with corresponding calcium compounds or with ammonia or a suitable organic amine, stoichiometric amounts or only a small excess of the salt-forming agent preferably being used. Acid addition salts of compounds of formula I are obtained in customary manner, e.g. by treating the compounds with an acid or a suitable anion exchange reagent. Internal salts of compounds of formula I containing acid and basic salt-forming groups, e.g. a free carboxy group and a free amino group, may be formed, e.g. by the neutralisation of salts, such as acid addition salts, to the isoelectric point, e.g. with weak bases, or by treatment with ion exchangers.

A salt of a compound of the formula I can be converted in customary manner into the free compound; metal and ammonium salts can be converted, for example, by treatment with suitable acids, and acid addition salts, for example, by treatment with a suitable basic agent. In both cases, suitable ion exchangers may be used.

Stereoisomeric mixtures, e.g. mixtures of diastereomers, can be separated into their corresponding isomers in a manner known per se by means of appropriate separation methods. Diastereomeric mixtures for example may be separated into their individual diastereomers by means of fractionated crystallization, chromatography, solvent distribution, and similar procedures. This separation may take place either at the level of one of the starting compounds or in a compound of formula I itself. Enantiomers may be separated through the formation of diastereomeric salts, for example by salt formation with an enantiomer-pure chiral acid, or by means of chromatography, for example by HPLC, using chromatographic substrates with chiral ligands.

Intermediates and final products can be worked up and/or purified according to standard methods, e.g. using chromatographic methods, distribution methods, (re-) crystallization, and the like.

Starting Materials

In the subsequent description of starting materials and intermediates and their synthesis, R1, R2, R2*, R3, R4, R5, R6, R7, R8, T, G, W, X₁, X₂, X₃, X₄, X₅, y, z and PG have the meanings given above or in the Examples for the respective starting materials or intermediates, if not indicated otherwise directly or by the context. Protecting groups, if not specifically mentioned, can be introduced and removed at appropriate steps in order to prevent functional groups, the reaction of which is not desired in the corresponding reaction step or steps, employing protecting groups, methods for their introduction and their removal are as described above or below, e.g. in the references mentioned under “General Process Conditions”. The person skilled in the art will readily be able to decide whether and which protecting groups are useful or required.

A compound of the formula II wherein R7 is hydrogen and the other moieties are as defined for compounds of the formula II can, for example, be prepared by reducing the double bond in a compound of the formula VI,

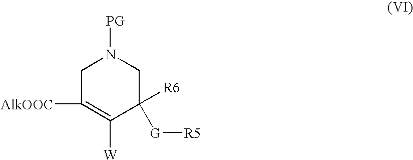

wherein Alk is unsubstituted or substituted alkyl, preferably C₁-C₇-alkyl, in the presence of an appropriate reductant, such as (i) hydrogen in the presence of a noble metal catalyst, e.g. in dispersion such as Pd on charcoal or with a homogenous catalyst such as Pd(OAc)₂, in an appropriate solvent, for example an alcohol, such as ethanol, or N-methylpyrrolidone, or mixtures of two or more thereof, at preferred temperatures in the range from 0 to 50° C., e.g. at room temperature; (ii) in the presence of a complex hydride, especially sodium boro-hydride, and e.g. NiCl₂ in an appropriate solvent, such as an alcohol, e.g. at temperatures from −30 to 30° C., or preferably in the presence of sodium bis(2-methoxyethoxy)aluminium hydride in toluene at preferred temperatures in the range from −50 to 20° C., e.g. from −40 to 0° C.; or (iii) in the presence of a reducing metal, such as Mg, in an appropriate solvent, e.g. an alcohol, such as methanol, at preferred temperatures from −20 to 40° C., resulting in a compound of the formula VII,

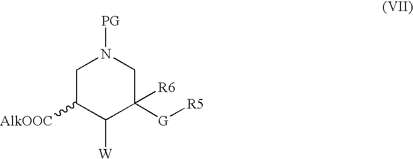

wherein Alk is as defined under compounds of the formula VI, which can then, if desired under epimerization to the corresponding compound of the formula II wherein the carboxy group and W are present in the configuration of the R1R2N-T- and the W in formula IA given above, preferably be hydrolyzed and thus converted to the corresponding compound of the formula II, e.g. (i) in the presence of an alcoholate of the formula MeOAlk, where Me is preferably an alkali metal, e.g. Na, and Alk is as defined under formula VI, in the presence of an appropriate solvent, e.g. the corresponding alcohol AIkOH, e.g. methanol or ethanol, to achieve epimerization, optionally followed by hydrolysis with water, e.g. at elevated temperatures from 30 to 80° C. or under reflux, or (ii) by addition of a metal hydroxide, e.g. potassium hydroxide or lithium hydroxide, in the presence of water at elevated temperatures, e.g. from 50° C. to the reflux temperature of the mixture. If desired or useful, protecting groups can be introduced before hydrolysis, e.g. for protecting amino or hydroxy G-R5, such as methoxymethyl or tert-butyldimethylsilyl.

A compound of the formula VI wherein G-R5 is OH can, for example, be prepared by reacting an epoxide of the formula VIII,

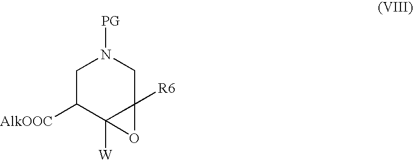

wherein Alk is as defined under formula VI in the presence of an alcoholate, such a sodium methanolate, in the corresponding alcohol, such as methanol, preferably at elevated temperatures e.g. between 50° C. and the reflux temperature, which results in the corresponding compound of the formula VI.

An OH group G-R5 e.g. in formula VII can also be converted into corresponding groups -G-R5 wherein G is thio, imino or substituted imino (—NR8-) as defined above according to reactions that are well known in the art (e.g. by nucleophilic substitution with a precursor of R5 carrying an SH or NH₂ or NHR8 group after e.g. transformation of an OH group G-R5 in formula VII to a halo or toluolsulfonyl or methysulfonyl group), or by addition to a compound of the formula VIII followed by dehydration.

A compound of the formula VIII can, for example, be prepared by epoxidation of a tetrahydropyridine of the formula IX,

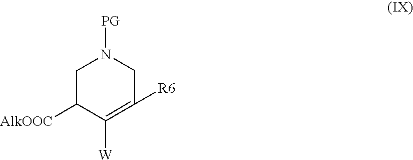

with an appropriate epoxidizing agent, such as an organic peroxo acid, especially m-chloroperbenzoic acid, in an appropriate solvent, such as a halogenated hydrocarbon, e.g. methylene chloride, at preferred temperatures from −20 to 20° C., e.g. at about 0° C., resulting in the corresponding epoxide of the formula VIII.

A compound of the formula IX can be obtained by shifting the double bond in a tetrahydropyridine of the formula X,

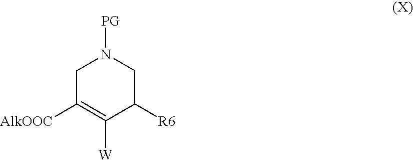

e.g. in the presence of a strong base, such as lithium diisopropylamide, in an appropriate solvent, e.g. hexamethylphosphoroamide and/or a cyclic ether, such as tetrahydrofurane, preferably at low temperatures, e.g. from −90 to −50° C.

A tetrahydropyridine compound of the formula X can, for example, be prepared by reacting a compound of the formula XI,

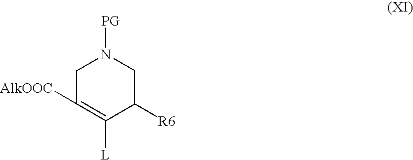

wherein L is as described above for a compound of the formula IV and the other moieties have the meanings described for a compound of the formula X, with a compound of the formula XII,

W-Q (XII)

wherein W is as described for a compound of the formula I and Q is —B(OH)₂ or a leaving group as defined for a compound of the formula V, under reaction conditions analogous to those described under reaction (b) above.

A compound of the formula X wherein W is a ring of the formula IC wherein X₁ is oxygen, X₂ is N, and each of X₃ and X₄ is CH and R3 is bound instead of the hydrogen at X₄ can be prepared by reaction of 4-R3-substituted phenyloxazole with a compound of the formula XI given above by first reacting the 4-R3-substituted phenyloxazole in the presence of a strong base, such as butyllithium, followed by treatment with zinc chloride, both in an appropriate solvent, such as tetrahydrofurane, at low temperatures e.g. from −90 to −50° C., followed by the addition of the compound of the formula XI and a catalyst, especially Pd(PPh₃)₄ in the same solvent and at appropriate temperatures, e.g. from −30 to 30° C., thus obtaining the corresponding compound of the formula X.

A compound of the formula X wherein W is a moiety of the formula IC wherein X₁ is O, X₂ is CH, X₃ is CH and X₄ is N and R3 is bound instead of the H at position X₃ can be prepared from a compound of the formula XI given above by reaction with trimethylsilyl-acetylene (Me₃-Si—C≡CH) in the presence e.g. of CuI and a tertiary nitrogen base, such as triethylamine, and a catalyst, e.g. Pd(PPh₃)₄, in an appropriate solvent, such as dimethylformamide, and at appropriate temperatures, e.g. from 30 to 70° C., to give the corresponding compound of the formula XIII,

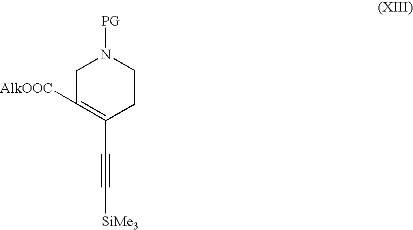

which is then reacted under desilylation, e.g. with cesium fluoride in an appropriate solvent, such as methanol and/or water, at an appropriate temperature, e.g. from 0 to 50° C., followed by reaction of the free acetylene compound (where in formula XIII instead of the SiMe₃ group a hydrogen is present) with an carboximidoylhalogenide of the formula XIV,

R3-C(═NH—OH)-HaI (XIV)

wherein HaI is halogen, especially chloro, in the presence of a nitrogen base, e.g. triethylamine, in an appropriate solvent, e.g. methylene chloride, and at appropriate temperatures, e.g. from 0 to 50° C.; thus obtaining the corresponding compound of the formula X with the ring IC as described.

In a compound of the formula X wherein W carries a nitro substituent at a position of R3, the nitro and the double bond in the tetrahydropyridine ring can be reduced to give an amino group and a piperidine ring, respectively, and then the amino can be converted into substituted amino e.g. by reaction with a complementary acid chloride under customary conditions, e.g. in the presence of a nitrogen base, such as triethylamine, in an appropriate solvent, e.g. methylene chloride, and at customary temperatures, e.g. from 0 to 50° C., thus yielding a corresponding compound of the formula X.

A compound of the formula XI is known or can be prepared according to methods that are known in the art, e.g. analogously to or as described in WO 04/002957 or US 2003/216441 or e.g. by halogenation, hydroboration or acylation (e.g. with tetrafluoroacetate anhydride) under customary reaction conditions starting from a corresponding compound wherein instead of L in formula XI a hydroxy group is present.

A compound of the formula VI wherein G is methylene (—CH₂—) (and R5 is preferably unsubstituted or substituted alkyl, such as C₁-C₇-alkoxy-C₁-C₇-alkyl) can be prepared starting form a compound of the formula XIA,

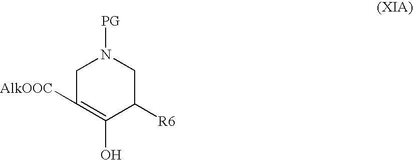

by first reacting it with a strong base, such as sodium hydride, in an appropriate solvent, such as tetrahydrofurane, preferably at lower temperatures, e.g. from −20 to 25° C., and then adding e.g. lithium diisopropylamide, in the presence of hexamethylphosphoroamide at the same temperature and a compound of the formula XXI;

R5-CH₂-HaI (XXI)

wherein R5 is as defined for a compound of the formula I, preferably as just mentioned before formula XIA, and HaI is a leaving group, especially halogen, at about the same temperatures; this results in a corresponding compound of the formula XIB,

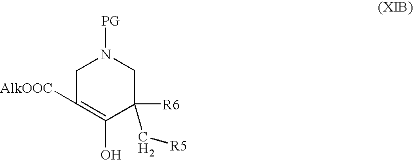

wherein R5 is as just defined which can then be reacted with an acylating agent introducing a leaving group L*, such as trifluoroacetic anhydride, or the like, in the presence of a tertiary amine, such as N,N-di(isopropyl)-N-ethylamine, in an appropriate solvent, such as dichloromethane, at preferably lower temperatures, e.g. from −20 to 15° C., to give a corresponding compound of the formula XIC,

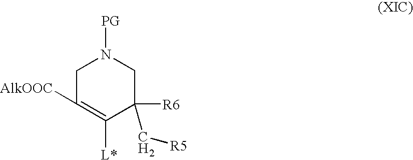

wherein R5 and L* are as just defined. This compound can then be reacted with a compound of the formula XII given above wherein Q is −B(OH)₂ in the presence of an appropriate catalyst, such as Pd(PPh₃)₄, in the presence of a base, such as potassium phosphate, in an appropriate solvent, such as dioxane in the presence of water, at temperatures e.g. from 0 to 90° C., to give the corresponding compound of the formula VI.

A compound of the formula IV can, for example, be prepared analogously to a compound of the formula I but using starting materials (e.g. corresponding to those of the formula II) wherein instead of W the moiety

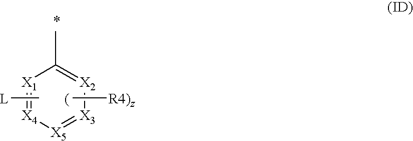

is present wherein the symbols have the meanings given under a compound of the formula IV, L is bound to a ring carbon and the asterisk denotes the point of binding to the rest of the molecule. The reaction conditions can then be analogous to those described under (a) used for the synthesis of compounds of the formula I, the starting materials can be analogous to those mentioned there as starting materials, e.g. analogues of the compounds of the formula X wherein instead of the moiety W one of the formula ID is present can be used. The reaction conditions can be as described for the other starting materials given hereinbefore.

Starting materials of the formula IV wherein L is hydroxy and the other symbols have the meanings given under formula IV can, for example, be prepared from the precursors wherein instead of hydroxy L a protected hydroxy is present by removal of the protecting group, e.g. in case of methoxymethyl by reaction with an acid, such as TFA, in an appropriate solvent, e.g. dichloromethane, for example at temperatures between 0 and 50° C. These precursors can be prepared in analogy to an analogue of a compound of the formula X and II or I wherein instead of the group W the moiety of the formula ID with protected hydroxy instead of L is present, preferably under conditions analogous to those for the corresponding compounds as given above.

Compounds of the formula III, wherein R2 is bound via methylene (as part of R2), can, for example, be prepared by reacting a compound of the formula XV,

R2a-CHO (XV)

(obtainable e.g. from the corresponding acids or their esters by reduction to a hydroxymethyl group and then oxidation to the —CHO group, e.g. by first reducing the carboxy function in the presence of an appropriate complex hydride, e.g. borane dimethylsulfide, in an appropriate solvent, e.g. tetrahydrofurane, at preferred temperatures between −20 and 40° C., to the corresponding hydroxymethylene group, and then oxidizing to the aldehyde group, for example in the presence of Dess Martin periodinane e.g. in methylene chloride and/or water or of 2,2,6,6,-tetramethyl-1-piperidinyloxy free radical e.g. in toluene and/or ethyl acetate in the presence of potassium bromide, water and potassium hydrogencarbonate, at preferred temperatures in the range from 0 to 50° C.) wherein R2a is a moiety that together with —CH₂— by which it is bound in formula III forms a corresponding moiety R2 in a compound of the formula I, under conditions of reductive amination, e.g. analogous to those described for the conversion with an aldehyde of the formula IIIA above, with an amine of the formula XVI,

R1-NH₂ (XVI)

wherein R1 is as defined for a compound of the formula I.

Alternatively, compounds of the formula III as described under reaction (b) above can be prepared by reaction of a compound of the formula XVII,

R2-LG (XVII)

wherein R2 is as defined for compounds of the formula I and LG is a leaving group, e.g. halo, under customary substitution reaction conditions, e.g. in the presence of a tertiary nitrogen base, such as triethylamine, in a customary solvent, e.g. tetrahydrofurane, e.g. at temperatures from 0 to 50° C. with a compound of the formula XVI as described above. Compounds of the formula XVII can be obtained from precursors wherein instead of LG hydroxy is present by introducing LG, e.g. by halogenation with halosuccinimides.

A compound of the formula III wherein R2 is hydrogen can be obtained from a nitro compound of the formula XVIII

R2-NO₂ (XVIII)

wherein R2 is as defined for a compound of the formula I by reduction with a reductant, such as a metal, especially iron, preferably in powder form, in the presence of an acid, e.g. hydrochloric acid, and an appropriate solvent, e.g. an alcohol, such as ethanol, e.g. at temperatures from 10° C. to the reflux temperature, e.g. at about 60° C.

A halo, e.g. bromo, group in place of Q in a compound of the formula XII or in place of L in a compound of the formula IV or in place of L in a compound of the formula XI can also be converted into the corresponding —B(OH)₂ group e.g. by reaction with a solution of an alkylalkalimetal, such as n-butyllithium, in an appropriate solvent, e.g. hydrocarbons, such as hexane, and/or tetrahydrofurane, first at lower temperatures, e.g. from −100 to −50° C., with subsequent addition of tri-lower alkylborane, e.g. (iPrO)₃B, and reaction at preferred temperatures from 0 to 50° C., thus yielding the corresponding starting materials.

In a starting maerial of the formula XV, a moiety R2a, or in a compound of the formula XVII or of the formula XVIII, a moiety R2, where R2a or R2 each are comprising a hydroxy group or (e.g. as part of a heterocyclic ring, such as indole) an imino (—NH—) group, the hydroxy or imino may be alkylated, e.g. with a C₁-C₇-alkoxy-C₁-C₇-alkyl moiety, by reaction with a corresponding alkyl-halogenide or -arylsulfonate, e.g. a C₁-C₇-alkoxy-C₁-C₇-alkyl-halogenide or -sulfonate, especially a corresponding bromide or iodide or tosylate, e.g. in the presence of potassium iodide, where required in the presence of a base, such as sodium hydride, in an appropriate solvent, such as N,N-di-(methyl)-formamide, e.g. at temperatures from −20 to 80° C., such as from 0 to 60° C., to give the corresponding alkylated starting material of the formula XV, XVII or XVIII, respectively.

Starting materials of the formula X can also be prepared from the corresponding oxo compounds of the formula XIX,

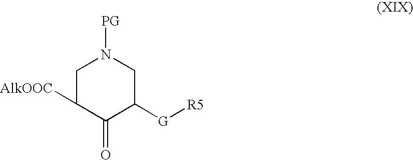

by reaction with a strong base, e.g. lithiuim diisopropylamide, in an appropriate solvent, e.g. tetrahydrofurane, at lower temperatures, e.g. from −30 to 20° C., followed by protection of the resulting hydroxy group, e.g. by reaction with methoxymethylchloride e.g. in the same reaction mixture at preferred temperatures from 0 to 50° C., and subsequent transformation of the hydroxy group into a group L, e.g. by reaction with trifluoroacetic acid anhydride in the presence of an appropriate base, e.g. diisopropylethylamine, in an appropriate solvent, such as dichloromethane, at preferred temperatures from −100 to −50° C.

Other starting materials, their synthesis or analogous methods for their synthesis are known in the art, commercially available, and/or they can be found in or derived from the Examples.

General Process Conditions

The following applies in general to all processes mentioned hereinbefore and hereinafter, while reaction conditions specifically mentioned above or below are preferred:

In any of the reactions mentioned hereinbefore and hereinafter, protecting groups may be used where appropriate or desired, even if this is not mentioned specifically, to protect functional groups that are not intended to take part in a given reaction, and they can be introduced and/or removed at appropriate or desired stages. Reactions comprising the use of protecting groups are therefore included as possible wherever reactions without specific mentioning of protection and/or deprotection are described in this specification.

Within the scope of this disclosure only a readily removable group that is not a constituent of the particular desired end product of formula I is designated a “protecting group”, unless the context indicates otherwise. The protection of functional groups by such protecting groups, the protecting groups themselves, and the reactions appropriate for their introduction and removal are described for example in standard reference works, such as J. F. W. McOmie, “Protective Groups in Organic Chemistry”, Plenum Press, London and New York 1973, in T. W. Greene and P. G. M. Wuts, “Protective Groups in Organic Synthesis”, Third edition, Wiley, New York 1999, in “The Peptides”; Volume 3 (editors: E. Gross and J. Meienhofer), Academic Press, London and New York 1981, in “Methoden der organischen Chemie” (Methods of Organic Chemistry), Houben Weyl, 4th edition, Volume 15/I, Georg Thieme Verlag, Stuttgart 1974, in H.-D. Jakubke and H. Jeschkeit, “Aminosäuren, Peptide, Proteine” (Amino acids, Peptides, Proteins), Verlag Chemie, Weinheim, Deerfield Beach, and Basel 1982, and in Jochen Lehmann, “Chemie der Kohlenhydrate: Monosaccharide and Derivate” (Chemistry of Carbohydrates: Monosaccharides and Derivatives), Georg Thieme Verlag, Stuttgart 1974. A characteristic of protecting groups is that they can be removed readily (i.e. without the occurrence of undesired secondary reactions) for example by solvolysis, reduction, photolysis or alternatively under physiological conditions (e.g. by enzymatic cleavage).

All the above-mentioned process steps can be carried out under reaction conditions that are known per se, preferably those mentioned specifically, in the absence or, customarily, in the presence of solvents or diluents, preferably solvents or diluents that are inert towards the reagents used and dissolve them, in the absence or presence of catalysts, condensation or neutralizing agents, for example ion exchangers, such as cation exchangers, e.g. in the H⁺ form, depending on the nature of the reaction and/or of the reactants at reduced, normal or elevated temperature, for example in a temperature range of from about −100° C. to about 190° C., preferably from approximately −80° C. to approximately 150° C., for example at from −80 to −60° C., at room temperature, at from −20 to 40° C. or at reflux temperature, under atmospheric pressure or in a closed vessel, where appropriate under pressure, and/or in an inert atmosphere, for example under an argon or nitrogen atmosphere.

The solvents from which those solvents that are suitable for any particular reaction may be selected include those mentioned specifically or, for example, water, esters, such as lower alkyl-lower alkanoates, for example ethyl acetate, ethers, such as aliphatic ethers, for example diethyl ether, or cyclic ethers, for example tetrahydrofurane or dioxane, liquid aromatic hydrocarbons, such as benzene or toluene, alcohols, such as methanol, ethanol or 1- or 2-propanol, nitriles, such as acetonitrile, halogenated hydrocarbons, e.g. as methylene chloride or chloroform, acid amides, such as dimethylformamide or dimethyl acetamide, bases, such as heterocyclic nitrogen bases, for example pyridine or N-methylpyrrolidin-2-one, carboxylic acid anhydrides, such as lower alkanoic acid anhydrides, for example acetic anhydride, cyclic, linear or branched hydrocarbons, such as cyclohexane, hexane or isopentane, or mixtures of these, for example aqueous solutions, unless otherwise indicated in the description of the processes. Such solvent mixtures may also be used in working up, for example by chromatography or partitioning.

The invention relates also to those forms of the process in which a compound obtainable as intermediate at any stage of the process is used as starting material and the remaining process steps are carried out, or in which a starting material is formed under the reaction conditions or is used in the form of a derivative, for example in protected form or in the form of a salt, or a compound obtainable by the process according to the invention is produced under the process conditions and processed further in situ. In the process of the present invention those starting materials are preferably used which result in compounds of formula I described as being preferred. Special preference is given to reaction conditions that are identical or analogous to those mentioned in the Examples. The invention relates also to novel starting compounds and intermediates described herein, especially those leading to compounds mentioned as preferred herein.

Pharmaceutical Use, Pharmaceutical Preparations and Methods

As described above, the compounds of the present invention are inhibitors of renin activity and, thus, may be employed for the treatment of hypertension, atherosclerosis, unstable coronary syndrome, congestive heart failure, cardiac hypertrophy, cardiac fibrosis, cardiomyopathy postinfarction, unstable coronary syndrome, diastolic dysfunction, chronic kidney disease, hepatic fibrosis, complications resulting from diabetes, such as nephropathy, vasculopathy and neuropathy, diseases of the coronary vessels, restenosis following angioplasty, raised intra-ocular pressure, glaucoma, abnormal vascular growth and/or hyperaldosteronism, and/or further cognitive impairment, alzheimers, dementia, anxiety states and cognitive disorders, and the like.

The present invention further provides pharmaceutical compositions comprising a therapeutically effective amount of a pharmacologically active compound of the instant invention, alone or in combination with one or more pharmaceutically acceptable carriers.

The pharmaceutical compositions according to the present invention are those suitable for enteral, such as oral or rectal, transdermal and parenteral administration to mammals, including man, to inhibit renin activity, and for the treatment of conditions associated with (especially inappropriate) renin activity. Such conditions include hypertension, atherosclerosis, unstable coronary syndrome, congestive heart failure, cardiac hypertrophy, cardiac fibrosis, cardiomyopathy postinfarction, unstable coronary syndrome, diastolic dysfunction, chronic kidney disease, hepatic fibrosis, complications resulting from diabetes, such as nephropathy, vasculopathy and neuropathy, diseases of the coronary vessels, restenosis following angioplasty, raised intra-ocular pressure, glaucoma, abnormal vascular growth and/or hyperaldosteronism, and/or further cognitive impairment, alzheimers, dementia, anxiety states and cognitive disorders and the like.

Thus, the pharmacologically active compounds of the invention may be employed in the manufacture of pharmaceutical compositions comprising an effective amount thereof in conjunction or admixture with excipients or carriers suitable for either enteral or parenteral application. Preferred are tablets and gelatin capsules comprising the active ingredient together with:

a) diluents, e.g., lactose, dextrose, sucrose, mannitol, sorbitol, cellulose and/or glycine;

b) lubricants, e.g., silica, talcum, stearic acid, its magnesium or calcium salt and/or polyethyleneglycol; for tablets also

c) binders, e.g., magnesium aluminum silicate, starch paste, gelatin, tragacanth, methylcellulose, sodium carboxymethylcellulose and or polyvinylpyrrolidone; if desired

d) disintegrants, e.g., starches, agar, alginic acid or its sodium salt, or effervescent mixtures; and/or

e) absorbants, colorants, flavors and sweeteners.

Injectable compositions are preferably aqueous isotonic solutions or suspensions, and suppositories are advantageously prepared from fatty emulsions or suspensions.

Said compositions may be sterilized and/or contain adjuvants, such as preserving, stabilizing, wetting or emulsifying agents, solution promoters, salts for regulating the osmotic pressure and/or buffers. In addition, they may also contain other therapeutically valuable substances. Said compositions are prepared according to conventional mixing, granulating or coating methods, respectively, and contain about 0.1-75%, preferably about 1-50%, of the active ingredient.

Suitable formulations for transdermal application include a therapeutically effective amount of a compound of the invention with carrier. Advantageous carriers include absorbable pharmacologically acceptable solvents to assist passage through the skin of the host. Characteristically, transdermal devices are in the form of a bandage comprising a backing member, a reservoir containing the compound optionally with carriers, optionally a rate controlling barrier to deliver the compound of the skin of the host at a controlled and pre-determined rate over a prolonged period of time, and means to secure the device to the skin.

Accordingly, the present invention provides pharmaceutical compositions as described above for the treatment of conditions mediated by renin activity, preferably, hypertension, atherosclerosis, unstable coronary syndrome, congestive heart failure, cardiac hypertrophy, cardiac fibrosis, cardiomyopathy postinfarction, unstable coronary syndrome, diastolic dysfunction, chronic kidney disease, hepatic fibrosis, complications resulting from diabetes, such as nephropathy, vasculopathy and neuropathy, diseases of the coronary vessels, restenosis following angioplasty, raised intra-ocular pressure, glaucoma, abnormal vascular growth and/or hyperaldosteronism, and/or further cognitive impairment, alzheimers, dementia, anxiety states and cognitive disorders, as well as methods of their use.

The pharmaceutical compositions may contain a therapeutically effective amount of a compound of the formula I as defined herein, either alone or in a combination with another therapeutic agent, e.g., each at an effective therapeutic dose as reported in the art. Such therapeutic agents include:

a) antidiabetic agents such as insulin, insulin derivatives and mimetics; insulin secretagogues such as the sulfonylureas, e.g., Glipizide, glyburide and Amaryl; insulinotropic sulfonylurea receptor ligands such as meglitinides, e.g., nateglinide and repaglinide; peroxisome proliferator-activated receptor (PPAR) ligands; protein tyrosine phosphatase-1B (PTP-1B) inhibitors such as PTP-112; GSK3 (glycogen synthase kinase-3) inhibitors such as SB-517955, SB-4195052, SB-216763, NN-57-05441 and NN-57-05445; RXR ligands such as GW-0791 and AGN-194204; sodium-dependent glucose cotransporter inhibitors such as T-1095; glycogen phosphorylase A inhibitors such as BAY R3401; biguanides such as metformin; alpha-glucosidase inhibitors such as acarbose; GLP-1 (glucagon like peptide-1), GLP-1 analogs such as Exendin-4 and GLP-1 mimetics; and DPPIV (dipeptidyl peptidase IV) inhibitors such as LAF237;

b) hypolipidemic agents such as 3-hydroxy-3-methyl-glutaryl coenzyme A (HMG-CoA) reductase inhibitors, e.g., lovastatin, pitavastatin, simvastatin, pravastatin, cerivastatin, mevastatin, velostatin, fluvastatin, dalvastatin, atorvastatin, rosuvastatin and rivastatin; squalene synthase inhibitors; FXR (farnesoid X receptor) and LXR (liver X receptor) ligands; cholestyramine; fibrates; nicotinic acid and aspirin;

c) anti-obesity agents such as orlistat; and

d) anti-hypertensive agents, e.g., loop diuretics such as ethacrynic acid, furosemide and torsemide; angiotensin converting enzyme (ACE) inhibitors such as benazepril, captopril, enalapril, fosinopril, lisinopril, moexipril, perinodopril, quinapril, ramipril and trandolapril; inhibitors of the Na-K-ATPase membrane pump such as digoxin; neutralendopeptidase (NEP) inhibittors; ACE/NEP inhibitors such as omapatrilat, sampatrilat and fasidotril; angiotensin II antagonists such as candesartan, eprosartan, irbesartan, losartan, telmisartan and valsartan, in particular valsartan; β-adrenergic receptor blockers such as acebutolol, atenolol, betaxolol, bisoprolol, metoprolol, nadolol, propranolol, sotalol and timolol; inotropic agents such as digoxin, dobutamine and milrinone; calcium channel blockers such as amlodipine, bepridil, diltiazem, felodipine, nicardipine, nimodipine, nifedipine, nisoldipine and verapamil; aldosterone receptor antagonists; and aldosterone synthase inhibitors.

Other specific anti-diabetic compounds are described by Patel Mona in Expert Opin Investig Drugs, 2003, 12(4), 623-633, in the FIGS. 1 to 7, which are herein incorporated by reference. A compound of the present invention may be administered either simultaneously, before or after the other active ingredient, either separately by the same or different route of administration or together in the same pharmaceutical formulation.

The structure of the therapeutic agents identified by code numbers, generic or trade names may be taken from the actual edition of the standard compendium “The Merck Index” or from databases, e.g., Patents International (e.g. IMS World Publications). The corresponding content thereof is hereby incorporated by reference.

Accordingly, the present invention provides pharmaceutical compositions comprising a therapeutically effective amount of a compound of the invention alone or in combination with a therapeutically effective amount of another therapeutic agent, preferably selected from anti-diabetics, hypolipidemic agents, anti-obesity agents or anti-hypertensive agents, most preferably from antidiabetics, anti-hypertensive agents or hypolipidemic agents as described above.

The present invention further relates to pharmaceutical compositions as described above for use as a medicament.

The present invention further relates to use of pharmaceutical compositions or combinations as described above for the preparation of a medicament for the treatment of conditions mediated by (especially inappropriate) renin activity, preferably, hypertension, atherosclerosis, unstable coronary syndrome, congestive heart failure, cardiac hypertrophy, cardiac fibrosis, cardiomyopathy postinfarction, unstable coronary syndrome, diastolic dysfunction, chronic kidney disease, hepatic fibrosis, complications resulting from diabetes, such as nephropathy, vasculopathy and neuropathy, diseases of the coronary vessels, restenosis following angioplasty, raised intra-ocular pressure, glaucoma, abnormal vascular growth and/or hyperaldosteronism, and/or further cognitive impairment, alzheimers, dementia, anxiety states and cognitive disorders, and the like.

Thus, the present invention also relates to a compound of formula I for use as a medicament, to the use of a compound of formula I for the preparation of a pharmaceutical composition for the prevention and/or treatment of conditions mediated by (especially inappropriate) renin activity, and to a pharmaceutical composition for use in conditions mediated by (especially inappropriate) renin activity comprising a compound of formula I, or a pharmaceutically acceptable salt thereof, in association with a pharmaceutically acceptable diluent or carrier material therefore.

The present invention further provides a method for the prevention and/or treatment of conditions mediated by (especially inappropriate) renin activity, which comprises administering a therapeutically effective amount of a compound of the present invention to a warm-blooded animal, especially a human, in need of such treatment.

A unit dosage for a mammal of about 50-70 kg may contain between about 1 mg and 1000 mg, advantageously between about 5-600 mg of the active ingredient. The therapeutically effective dosage of active compound is dependent on the species of warm-blooded animal (especially mammal, more especially human), the body weight, age and individual condition, on the form of administration, and on the compound involved.

In accordance with the foregoing the present invention also provides a therapeutic combination, e.g., a kit, kit of parts, e.g., for use in any method as defined herein, comprising a compound of formula I, or a pharmaceutically acceptable salt thereof, to be used concomitantly or in sequence with at least one pharmaceutical composition comprising at least another therapeutic agent, preferably selected from anti-diabetic agents, hypolipidemic agents, anti-obesity agents or anti-hypertensive agents. The kit may comprise instructions for its administration.

Similarly, the present invention provides a kit of parts comprising: (i) a pharmaceutical composition comprising a compound of the formula I according to the invention; and (ii) a pharmaceutical composition comprising a compound selected from an anti-diabetic, a hypolipidemic agent, an anti-obesity agent, an anti-hypertensive agent, or a pharmaceutically acceptable salt thereof, in the form of two separate units of the components (i) to (ii).

Likewise, the present invention provides a method as defined above comprising co-administration, e.g., concomitantly or in sequence, of a therapeutically effective amount of a compound of formula I, or a pharmaceutically acceptable salt thereof, and at least a second drug substance, said second drug substance preferably being an anti-diabetic, a hypolipidemic agent, an anti-obesity agent or an anti-hypertensive agent, e.g., as indicated above.

Preferably, a compound of the invention is administered to a mammal in need thereof.

Preferably, a compound of the invention is used for the treatment of a disease which responds to a modulation of (especially inappropriate) renin activity.

Preferably, the condition associated with (especially inappropriate) renin activity is selected from hypertension, atherosclerosis, unstable coronary syndrome, congestive heart failure, cardiac hypertrophy, cardiac fibrosis, cardiomyopathy postinfarction, unstable coronary syndrome, diastolic dysfunction, chronic kidney disease, hepatic fibrosis, complications resulting from diabetes, such as nephropathy, vasculopathy and neuropathy, diseases of the coronary vessels, restenosis following angioplasty, raised intra-ocular pressure, glaucoma, abnormal vascular growth and/or hyperaldosteronism, and/or further cognitive impairment, alzheimers, dementia, anxiety states and cognitive disorders.

Finally, the present invention provides a method or use which comprises administering a compound of formula I in combination with a therapeutically effective amount of an anti-diabetic agent, a hypolipidemic agent, an anti-obesity agent or an anti-hypertensive agent.

Ultimately, the present invention provides a method or use which comprises administering a compound of formula I in the form of a pharmaceutical composition as described herein.

The above-cited properties are demonstrable in vitro and in vivo tests using advantageously mammals, e.g., mice, rats, rabbits, dogs, monkeys or isolated organs, tissues and preparations thereof. Said compounds can be applied in vitro in the form of solutions, e.g., preferably aqueous solutions, and in vivo either enterally, parenterally, advantageously intravenously, e.g., as a suspension or in aqueous solution. The concentration level in vitro may range between about 10⁻³ molar and 10⁻¹⁰ molar concentrations. A therapeutically effective amount in vivo may range depending on the route of administration, between about 0.001 and 500 mg/kg, preferably between about 0.1 and 100 mg/kg.

As described above, the compounds of the present invention have enzyme-inhibiting properties. In particular, they inhibit the action of the natural enzyme renin. Renin passes from the kidneys into the blood where it effects the cleavage of angiotensinogen, releasing the decapeptide angiotensin I which is then cleaved in the lungs, the kidneys and other organs to form the octapeptide angiotensin II. The octapeptide increases blood pressure both directly by arterial vasoconstriction and indirectly by liberating from the adrenal glands the sodium-ion-retaining hormone aldosterone, accompanied by an increase in extracellular fluid volume which increase can be attributed to the action of angiotensin II. Inhibitors of the enzymatic activity of renin lead to a reduction in the formation of angiotensin I, and consequently a smaller amount of angiotensin II is produced. The reduced concentration of that active peptide hormone is a direct cause of the hypotensive effect of renin inhibitors.

The action of renin inhibitors may be demonstrated inter alia experimentally by means of in vitro tests, the reduction in the formation of angiotensin I being measured in various systems (human plasma, purified human renin together with synthetic or natural renin substrate).

Inter alia the following in vitro tests may be used:

Recombinant human renin (expressed in Chinese Hamster Ovary cells and purified using standard methods) at 7.5 nM concentration is incubated with test compound at various concentrations for 1 h at RT in 0.1 M Tris-HCl buffer, pH 7.4, containing 0.05 M NaCl, 0.5 mM EDTA and 0.05% CHAPS. Synthetic peptide substrate Arg-Glu(EDANS)-Ile-His-Pro-Phe-His-Leu-Val-Ile_His_Thr-Lys(DABCYL)-Arg9 is added to a final concentration of 2 μM and increase in fluorescence is recorded at an excitation wave-length of 350 nm and at an emission wave-length of 500 nm in a microplate spectro-fluorimeter. IC50 values are calculated from percentage of inhibition of renin activity as a function of test compound concetration (Fluorescence Resonance Energy Transfer, FRET, assay). Compounds of the formula I, in this assay, preferably can show IC₅₀ values in the range from 1 nM to 20 μM.

Alternatively, recombinant human renin (expressed in Chinese Hamster Ovary cells and purified using standard methods) at 0.5 nM concentration is incubated with test compound at various concentrations for 2 h at 37° C. in 0.1 M Tris-HCl buffer, pH 7.4, containing 0.05 M NaCl, 0.5 mM EDTA and 0.05% CHAPS. Synthetic peptide substrate Arg-Glu(EDANS)-Ile-His-Pro-Phe-His-Leu-Val-Ile_His_Thr-Lys(DABCYL)-Arg9 is added to a final concentration of 4 μM and increase in fluorescence is recorded at an excitation wave-length of 340 nm and at an emission wave-length of 485 nm in a microplate spectro-fluorimeter. IC50 values are calculated from percentage of inhibition of renin activity as a function of test compound concentration (Fluorescence Resonance Energy Transfer, FRET, assay). Compounds of the formula I, in this assay, preferably can show IC₅₀ values in the range from 1 nM to 20 μM.

In another assay, human plasma spiked with recombinant human renin (expressed in Chinese Hamster Ovary cells and purified using standard methods) at 0.8 nM concentration is incubated with test compound at various concentrations for 2 h at 37° C. in 0.1 M Tris/HCl pH 7.4 containing 0.05 M NaCl, 0.5 mM EDTA and 0.025% (w/v) CHAPS. Synthetic peptide substrate Ac-Ile-His-Pro-Phe-His-Leu-Val-Ile-His-Asn-Lys-[DY-505-X5] is added to a final concentration of 2.5 μM. The enzyme reaction is stopped by adding an excess of a blocking inhibitor. The product of the reaction is separated by capillary electrophoresis and quantified by spectrophotometric measurement at 505 nM wave-length. IC50 values are calculated from percentage of inhibition of renin activity as a function of test compound concentration. Compounds of the formula I, in this assay, preferably can show IC₅₀ values in the range from 1 nM to 20 μM.

In another assay, recombinant human renin (expressed in Chinese Hamster Ovary cells and purified using standard methods) at 0.8 nM concentration is incubated with test compound at various concentrations for 2 h at 37° C. in 0.1 M Tris/HCl pH 7.4 containing 0.05 M NaCl, 0.5 mM EDTA and 0.025% (w/v) CHAPS. Synthetic peptide substrate Ac-Ile-His-Pro-Phe-His-Leu-Val-Ile-His-Asn-Lys-[DY-505-X5] is added to a final concentration of 2.5 μM. The enzyme reaction is stopped by adding an excess of a blocking inhibitor. The product of the reaction is separated by capillary electrophoresis and quantified by spectrophotometric measurement at 505 nM wave-length. IC50 values are calculated from percentage of inhibition of renin activity as a function of test compound concentration. Compounds of the formula I, in this assay, preferably show IC₅₀ values in the range from 1 nM to 20 μM.

In animals deficient in salt, renin inhibitors bring about a reduction in blood pressure. Human renin may differ from the renin of other species. In order to test inhibitors of human renin, primates, e.g., marmosets (Callithrix jacchus) may be used, because human renin and primate renin are substantially homologous in the enzymatically active region. Inter alia the following in vivo tests may be used:

Compounds can be tested in vivo in primates as described in the literature (see for example by Schnell C R et al. Measurement of blood pressure and heart rate by telemetry in conscious, unrestrained marmosets. Am J Physiol 264 (Heart Circ Physiol 33). 1993: 1509-1516; or Schnell C R et al. Measurement of blood pressure, heart rate, body temperature, ECG and activity by telemetry in conscious, unrestrained marmosets. Proceedings of the fifth FELASA symposium: Welfare and Science. Eds BRIGHTON. 1993.

### Examples

The following examples serve to illustrate the invention without limiting the scope thereof:

In the following examples the central piperidine ring is displayed in a specific configuration. However, this is intended to include also the compound that is the mirror image with regard to the substituents at this central piperidine ring. In other terms, if not mentioned otherwise a compound of formula I or a precursor thereof is in fact present as a mixture of the shown compound and the mirror image with regard to the substituents bound at the central piperidine ring (where no other chiral centers are present, the examples are thus enantiomeric mixtures, especially racemates). For example, where the central trisubstituted piperidine is represented in the following configuration

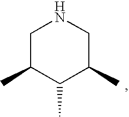

this is also intended to include the mirror image of the formula

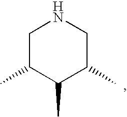

and the like. In other terms, if no other chiral groups are present, a compound of the formula I or a precursor thereof is a racemate. If further chiral groups are present, diastereomeric or enantiomeric mixtures may be present.

The same is also true for intermediates and starting materials, if not indicated otherwise or suggested otherwise by the context.

In any case, however, essentially pure compounds of the formula I wherein the essentially pure compound in the configuration as displayed is present are an especially preferred embodiment of the invention. They can be obtained e.g. according to standard procedures for the separation of enantiomers.

HPLC Conditions:

Column: Nucleosil 100-3 C18 HD, 125×4.0 mm.

Flow rate: 1.0 ml/min

Mobile phase: A) TFA/water (0.1/100, v/v), B) TFA/acetonitrile (0.1/100,v/v)

Gradient: linear gradient from 20% B to 100% B in 7 min

Detection: UV at 254 nm

Abbreviations

| | |
|---|---|
| Ac | acetyl |
| AcOH | acetic acid |
| AcONH₄ | ammonium acetate |
| aq | aqueous |
| Boc | tert-butoxycarbonyl |
| brine | sodium chloride solution saturated at RT |
| Celite | filtering aid based on diatomaceous earth |
| | (Celite ®, The Celite Corporation) |
| DAST | (diethylamino)sulfur trifluoride |
| DCM | dichloromethane |
| Dess-Martin | 1,1,1-triacetoxy-1,1-dihydro-1,2-benziodoxol- |
| reagent = Dess | 3(1H)-one - based reagent |
| Martin periodinane | |
| DIEA or DIPEA | N,N-diisopropylethylamine |
| DMF | N,N-dimethylformamide |
| EDC | 1-(3-dimethylaminopropyl)-3- |
| | ethylcarbodiimide hydrochloride |
| ES-MS | electrospray mass spectrometry |
| Et | ethyl |
| Fmoc | fluoren-9-ylmethyl |
| h | hour(s) |
| HMPA | hexamethylphosphoramide |
| HOAt | 1-hydroxy-7-azabenzotriazole |
| HPLC | high performance liquid chromatography |
| iPr | isopropyl |
| LAH | lithium aluminiumhydride |
| LDA | lithium diisopropylamide |
| m-CPBA | 3-chloroperbenzoic acid |
| Me | methyl |
| min | minute(s) |
| MOMCl | methoxymethylchloride |
| MS | mass spectrometry |
| MsCl | mesityl chloride |
| MS3A | molecular sieve 3A |
| NBS | N-bromosuccinimide |
| NMR | Nuclear Magnetic Resonance Spectroscopy |
| Ph | phenyl |
| Red-Al | Red-Al ® sodium bis(2- |
| | methoxyethoxy)aluminium hydride in |
| | toluene (Sigma Aldrich Co.) |
| Rf | ratio of fronts (ratio of running distance of |
| | analyte to distance of solvent front from starting |
| | point, respectively) |
| RP | reversed phase |
| RT or rt | room temperature |
| sat. | saturated |
| L-Selectride | L-Selectride ® = L-lithium tri-sec- |
| | butylborohydride (Sigma-Aldrich Co.) |
| TBAF | tetrabutylammonium fluoride |
| TBDMS | tert-butyl-dimethylsilyl |
| Tf₂O | trifluoroacetic anhydride |
| THF | tetrahydrofurane |
| TMS | trimethylsilyl |
| TMSOTf | trifluoromethanesulfonic acid-trimethylsilyl-estert |
| Bu | tert-butyl |
| Tf | triflate |
| t_{Re t} | retention time |
| WSCD | =EDC |

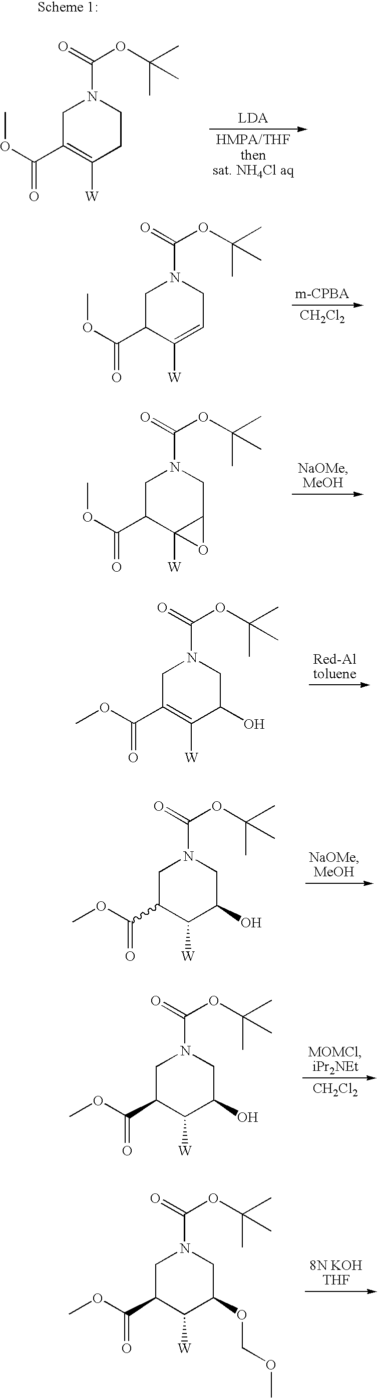

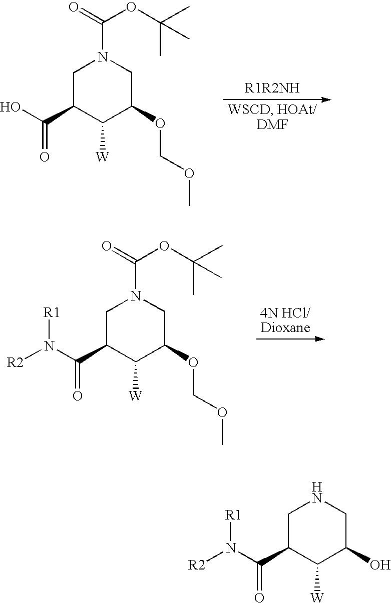

W, R1 and R2 are as defined for the corresponding moieties in formula I in the Examples 1 to 18.

### Example 1

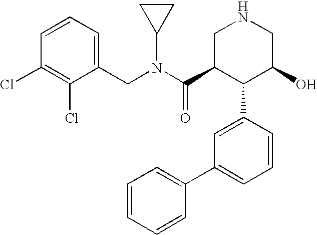

A mixture of Intermediate 1.1 (143 mg, 0.22 mmol) and a 4N dioxane solution of HCl (3 mL) is stirred under N₂ at RT. After stirring for 20 min, the reaction mixture is concentrated under reduced pressure to give the title compound of Example 1 as white solid; ES-MS: [M+H]⁺=495; HPLC: t_{Ret}=3.54 min.

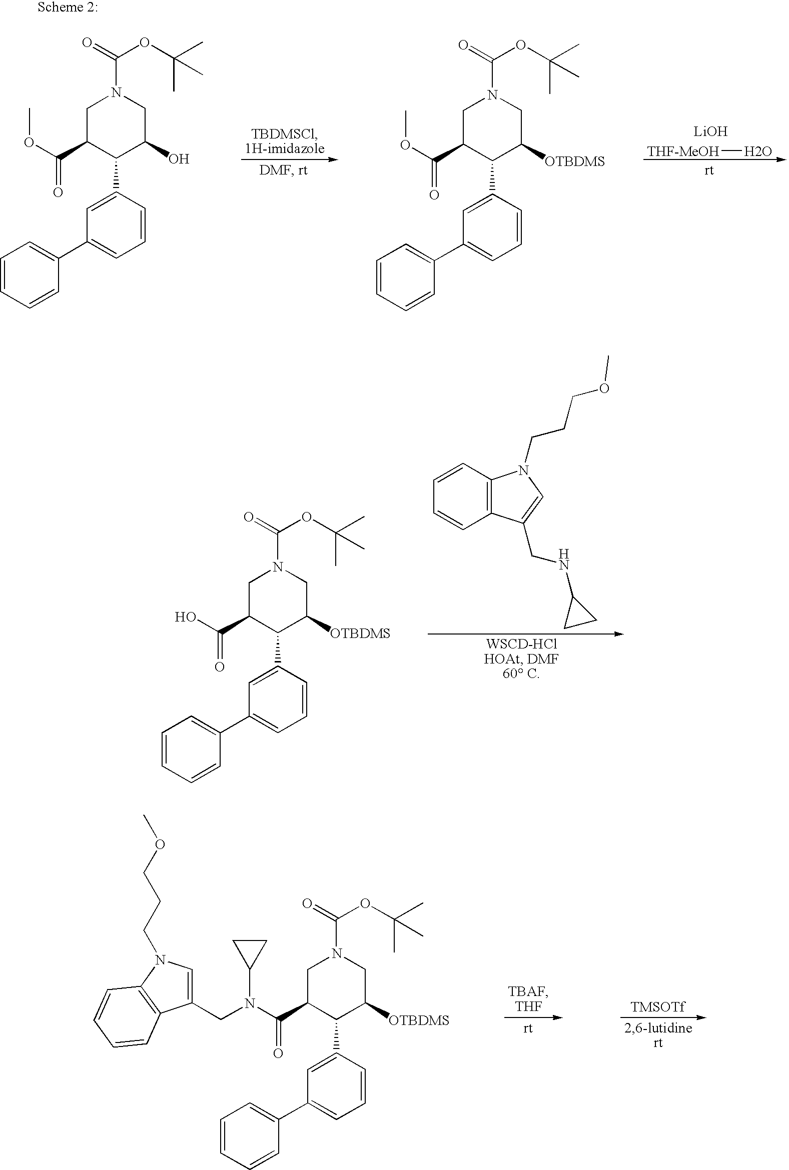

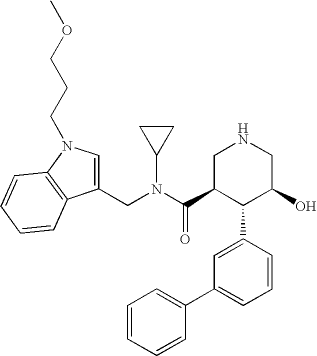

### Example 2

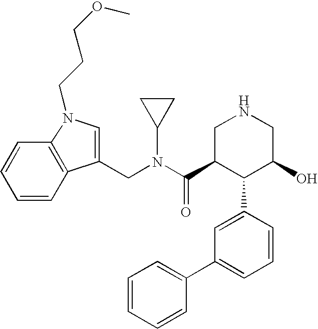

A mixture of compound of Intermediate 2.1 (75 mg, 0.10 mmol) and 1M TBAF in THF (0.15 mL, 0.15 mmol) in THF is stirred at RT. After adding H₂O, the reaction mixture is extracted with EtOAc. The combined organic phases are washed with H₂O and dried (MgSO₄), and then concentrated under reduced pressure. Without purification, a mixture of the resulting solution, TMSOTf (56 mg, 0.25 mmol) and 2,6-lutidine (54 mg, 0.50 mmol) in CH₂Cl₂ is stirred at RT for 0.5 h. After that, a small amount of saturated NaHCO₃ solution is added, and the resulting mixture is then concentrated under reduced pressure. The resulting solution is purified by RP-HPLC to give Example 2 as colorless oil; ES-MS: [M+H]⁺=537; HPLC: t_{Ret}=5.32 min.

### Example 3

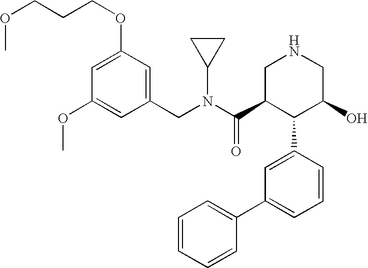

The compound of Example 3 is synthesized by deprotection of Intermediate 3.1 (120 mg, 0.27 mmol) analogously to the preparation of Example 1. MS: [M+1]⁺=545; HPLC: t_{Ret}=3.47 min.

The starting materials for Examples 1 to 3 are prepared as described in the following:

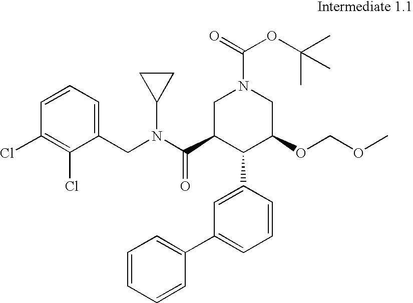

A mixture of compound of Intermediate 1.2 (170 mg, 0.39 mmol), cyclopropyl-(2,3-dichloro-benzyl)-amine (WO 03/093267) (166 mg, 0.77 mmol), EDC (152 mg, 0.77 mmol) and HOAt (152 mg, 0.77 mmol) in DMF (10 mL) is stirred under N₂ at RT for 10 h and then stirred at 65° C. for 2 h. After adding H₂O, the reaction mixture is extracted with EtOAc. The combined organic phases are washed with H₂O and brine and dried (MgSO₄), and then concentrated under reduced pressure. The resulting solution is purified by silica gel flash chromatography to give Intermediate 1.1 as white amorphous material; 1H-NMR (CDCl₃) δ; 0.49-0.75 (m, 2H), 0.79-0.97 (m, 2H), 1.38 (s, 3H), 1.52 (s, 6H), 2.36-2.46 (m, 1H), 2.80 (s, 3H), 2.90-3.09 (m, 1H), 3.18-3.52 (m, 2H), 3.78 (brt, 1H), 3.85-4.10 (m, 2H), 4.23 (d, 1H), 4.53 (d, 1H), 4.79-5.02 (m, 2H), 5.21-5.53 (m, 1H), 5.62-5.75 (m, 1H), 6.61 (t, 1H), 7.10-7.15 (m, 1H), 7.29-7.55 (m, 9H). Rf=0.44 (hexane/EtOAc 1:1).

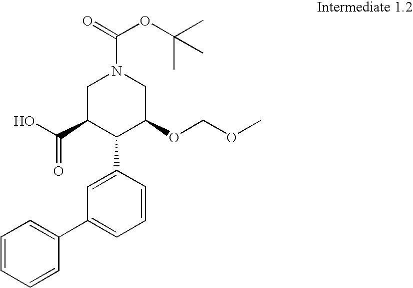

A solution of Intermediate 1.3 (240 mg, 0.53 mmol) in THF (10 mL)/MeOH (3 mL)/H₂O (2 mL) and 8N KOH solution (1 mL) is refluxed under N₂ for 15 h. After cooling down to RT, the solvent is removed in vacuo, and to the reside Et₂O (30 mL) and H₂O (20 mL) are added. The mixture is adjusted to weak acidic pH by slowly adding 1N HCl solution, and the resulting solution is then extracted with Et₂O (30 mL, 2×). The combined organic phases are washed with H₂O, brine and dried (MgSO₄) and then concentrated under reduced pressure to give Intermediate 1.2 as white amorphous material; ES-MS: [M+H−tBu]⁺=356; HPLC: t_{Ret}=4.30 min.

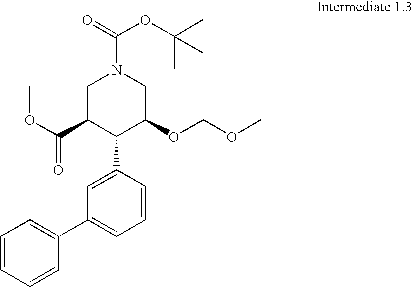

To a solution of Intermediate 1.4 (411 mg, 1.00 mmol) in DIEA (5 mL) and DCM (10 mL), MOMCI (0.15 mL, 2.00 mmol) is added at 0° C. After stirring at RT for 10 h and adding H₂O (15 mL), the reaction mixture is extracted with DCM (30 mL, 2×). The combined organic phases are washed with H₂O, brine and dried (MgSO₄), then concentrated under reduced pressure and subjected to silica gel flash chromatography to give Intermediate 1.3 as a yellow amorphous material; ES-MS: [M+H−tBu]⁺=400; HPLC: t_{Ret}=4.84 min.

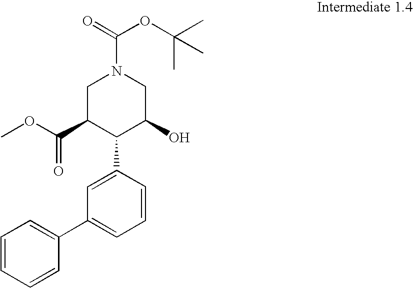

A solution of Intermediate 1.5 (283 mg, 0.69 mmol) and NaOMe in MeOH (56 mg, 1.03 mmol) in MeOH (1:1, 50 mL) is refluxed at 95° C. for 10 h. The solvent is removed in vacuo, and the residue is suspended in DCM and saturated NaHCO₃ solution. The organic layer is washed with H₂O and brine, dried over MgSO₄ and subjected to silica gel flash chromatography to give Intermediate 1.4 as a colorless amorphous material; ES-MS: [M+H−tBu]⁺=356; HPLC: t_{Ret}=3.87 min.

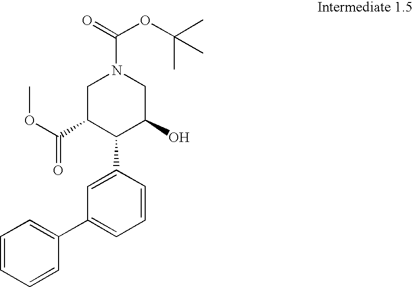

A mixture of Intermediate 1.6 (400 mg, 0.97 mmol) in toluene (15 mL) is cooled to −40° C. To the cooled solution, dropwise over 10 min 65% Red-Al (0.47 mL, 1.47 mmol) in toluene is added while maintaining the internal temperature at <−30° C. The reaction mixture is allowed to slowly warm to 0° C. over 2.5 hours and then added to a solution of saturated NaHSO₄ solution (5 mL). After adding Et₂O (50 mL) and H₂O (35 mL), the reaction mixture is extracted with Et₂O (30 mL, 2×). The combined organic phases are washed with H₂O, brine and dried (MgSO₄), then concentrated under reduced pressure and subjected to silica gel flash chromatography to give Intermediate 1.5 as a colorless amorphous material; ES-MS: [M+H−tBu]⁺=356; HPLC: t_{Ret}=4.17 min.

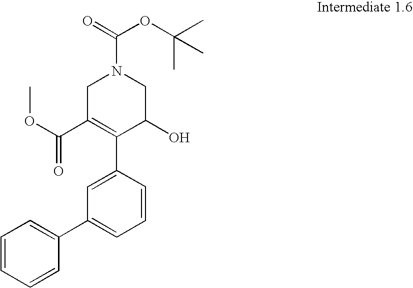

A mixture of Intermediate 1.7 (128 mg, 0.31 mmol) and NaOMe (25 mg, 0.47 mmol) in MeOH (15 mL) is refluxed at 95° C. for 2 h. After cooling down to RT, the reaction mixture is concentrated under reduced pressure. After adding saturated NaHCO₃ solution (15 mL), the reaction mixture is extracted with DCM (30 mL, 2×). The combined organic phases are washed with H₂O, brine and dried (MgSO₄), then concentrated under reduced pressure and subjected to silica gel flash chromatography to give Intermediate 1.6 as a colorless amorphous material; 1H-NMR (CDCl₃) δ1.52 (s, 9H), 1.90 (brs, 1H), 3.56 (s, 3H), 3.54-3.61 (m, 1H), 3.39-3.99 (m, 1H), 4.02-4.12 (m, 1H), 4.43-4.59 (m, 2H), 7.20 (d, 1H), 7.35 (t, 1H), 7.40-7.49 (m, 4H), 7.54-7.62 (m, 3H) Rf=0.19 (hexane/EtOAc 3:1).

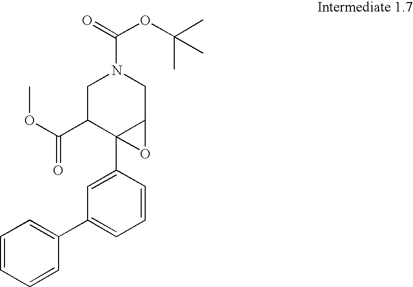

To a solution of Intermediate 1.8 (155 mg, 0.39 mmol) in DCM (10 mL), m-CPBA (243 mg, 0.99 mmol) is added at 0° C. After stirring at RT for 10 h and adding saturated NaHCO₃ solution (15 mL) and Na₂S₂O₃ solution (15 mL) at 0° C., the reaction mixture is extracted with DCM (30 mL, 2×). The combined organic phases are washed with H₂O, brine and dried (MgSO₄), concentrated under reduced pressure and subjected to silica gel flash chromatography to give Intermediate 1.7 as a mixture of diastereomers in the form of a colorless amorphous material; 1H-NMR (CDCl₃) δ 1.43-1.52 (m, 9H), 2.52-2.60 (m, 0.7H), 3.15-3.20 (m, 0.3H), 3.35-3.79 (m, 3H), 3.50 (s, 1H), 3.58 (s, 2H), 4.02-4.38 (m, 2H), 7.32-7.61 (m, 9H). Rf=0.33 (hexane/EtOAc 3:1).

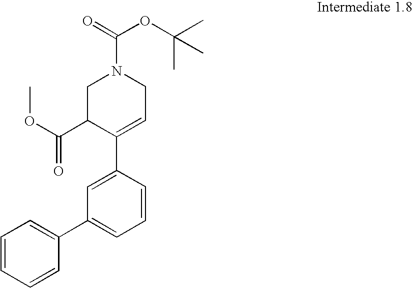

To a mixture of 2M THF solution of LDA (0.26 mL, 0.52 mmol) and HMPA (0.01 mL, 0.52 mmol) in THF (3 mL), a solution of Intermediate 1.9 (185 mg, 0.47 mmol) in THF (5 mL) is added under N₂ at −78° C. and kept under N₂ for 5 min. The reaction mixture is stirred at −78° C. for 1 h, and is then added to saturated NH₄Cl solution (15 mL) at 0° C. for 10 min. After adding H₂O, the reaction mixture is extracted with Et₂O (30 mL, 2×). The combined organic phases are washed with H₂O and brine and then dried (MgSO₄), concentrated under reduced pressure and subjected to silica gel flash chromatography to give Intermediate 1.8 as a colorless oil; 1H-NMR (400 MHz, CDCl₃) δ 1.48 (s, 9H), 3.33-3.46 (m, 1H), 3.58 (s, 3H), 3.69-3.78 (m, 1H), 3.82-3.99 (m, 1H), 4.32-4.56 (m, 2H), 6.20-6.30 (m, 1H), 7.29-7.60 (m, 9H). Rf=0.33 (hexane/EtOAc 3:1).

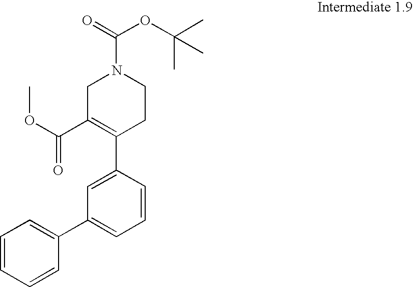

A mixture of 4-trifluoromethanesulfonyloxy-5,6-dihydro-2H-pyridine-1,3-dicarboxylic acid 1-tert-butyl ester 3-methyl ester (14 g, 36 mmol) reported by (WO 04/002957 or US2003/216441), 3-biphenylboronic acid (11.9 g, 43 mmol), K₃PO₄ (15.3 g, 72 mmol) and Pd(PPh₃)₄ (1.25 g, 1.1 mmol) in dioxane (150 mL) are stirred under N₂ at 80° C. for 5 hours. After adding H₂O, the reaction mixture is extracted with EtOAc. The combined organic phases are washed with H₂O, brine and dried (Na₂SO₄), concentrated under reduced pressure and silica gel flash chromatography to give Intermediate 1.9 as white amorphous material; ES-MS: [M+H]⁺=394; HPLC: t_{Ret}=5.12 min

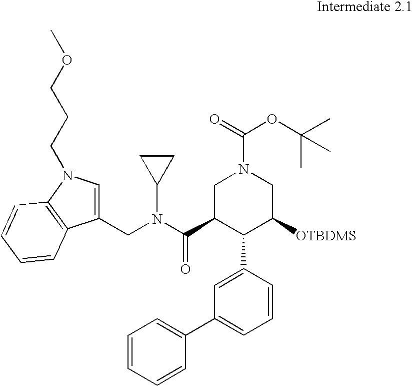

The compound of Intermediate 2.1 (75 mg, 0.10 mmol, 20%) is synthesized by condensation of Intermediate Intermediate 2.2 (120 mg, 0.27 mmol) with Intermediate 2.4 (193 mg, 0.75 mmol) analogously to Intermediate 1.1. MS: [M+1]⁺=752; HPLC: t_{Ret}=6.53 min.

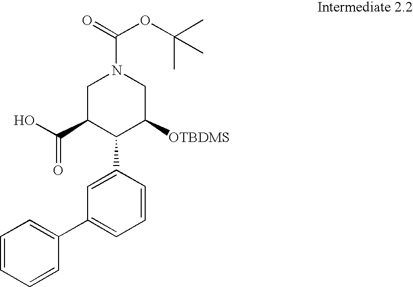

A mixture of compound of Intermediate 2.3 (220 mg, 0.41 mmol) and 2M LiOH (1.3 mL, 2.6 mmol) in THF-MeOH (1:1, 3 mL) is stirred at RT. After stirring for 1 h at RT, 2M LiOH (2.6 mL, 5.2 mmol) in THF-MeOH (1:1, 3 mL) is added to the reaction mixture. The reaction mixture is stirred at RT overnight and acidified by citric acid solution. The reaction mixture is extracted with EtOAc. The combined organic phases are washed with H₂O and dried (MgSO₄), and then concentrated under reduced pressure to give Intermediate 2.2 as colorless oil; ES-MS: [M+H−tBu]⁺=456; HPLC: t_{Ret}=5.92 min.

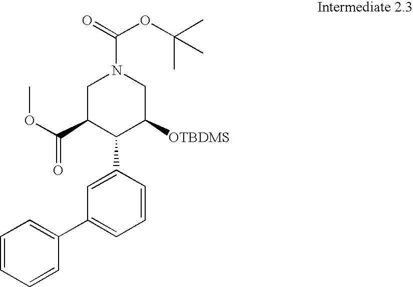

A mixture of compound of Intermediate 1.4 (300 mg, 0.73 mmol), TBDMSCI (131 mg, 0.87 mmol), and 1H-imidazole (74 mg, 1.1 mmol) in DMF (3 mL) is stirred at RT for 6 h. After adding H₂O, the reaction mixture is extracted with EtOAc. The combined organic phases are washed with H₂O and dried (MgSO₄), and then concentrated under reduced pressure. The resulting solution is purified by silica gel flash chromatography to give Intermediate 2.3 as colorless oil (220 mg, 0.41 mmol, 57%); ES-MS: [M+H−tBu]⁺=470; HPLC: t_{Ret}=6.28 min.

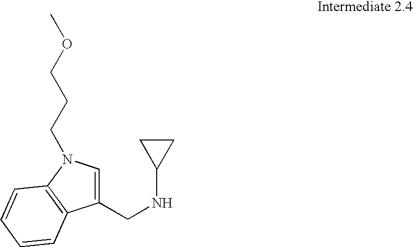

A mixture of Intermediate 2.5 (780 mg, 3.6 mmol), cyclopropylamine (410 mg, 7.2 mmol), AcOH (0.5 mL) and NaBH(OAc)₃ (1.1 g, 5.4 mmol) in DCM (3 mL) and MeOH (1 mL) is stirred under N₂ at 0° C. After stirring at RT for 1 hour, the reaction mixture is quenched with saturated aqueous NaHCO₃ and extracted with DCM. The combined organic phases are washed with H₂O, brine and dried (Na₂SO₄). Concentration under reduced pressure and silica gel flash chromatography give Intermediate 2.4 as yellow oil; ES-MS: [M+H]⁺=202; HPLC: t_{Ret}=2.67 min

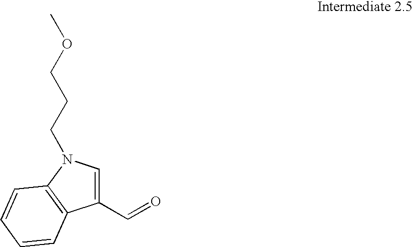

To a mixture of indole-3-carboxaldehyde (1.0 g, 6.9 mmol), toluene-4-sulfonic acid 3-methoxy-propyl ester (2.1 g, 9.0 mmol) and KI (1.1 g, 7.0 mmol) in DMF (15 mL), NaH (320 mg, 7.5 mmol) is added under N₂ at 0° C. After stirring at 50° C. for 4 h, the reaction mixture is supplemented with H₂O and extracted with EtOAc. The combined organic phases are washed with H₂O, brine and dried (Na₂SO₄). Concentration under reduced pressure and silica gel flash chromatography give Intermediate 2.5 as colorless oil; ES-MS: [M+H]⁺=218, HPLC: t_{Ret}=3.18 min.

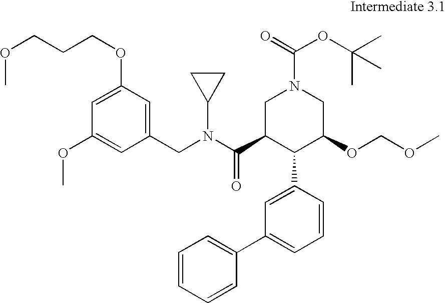

Intermediate 3.1 is synthesized by condensation of Intermediate 1.2 (120 mg, 0.27 mmol) with Intermediate 3.2 (273 mg, 1.09 mmol) analogously to the preparation of Intermediate 1.1. White amorphous material; ES-MS: [M+H]⁺=689; HPLC: t_{Ret}=5.22 minutes.

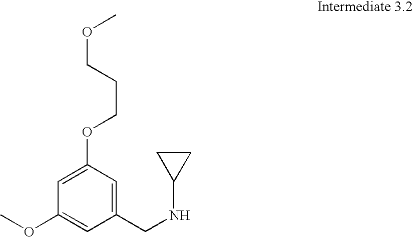

Intermediate 3.2 is synthesized by reductive amination of Intermediate 3.3 (2.50 g, 11.1 mmol) and cyclopropylamine (1.16 mL, 16.7 mmol) analogously to the preparation of Intermediate 2.4 using NaBH₃CN as a reducing agent. Yellow oil; ES-MS: [M+H]⁺=266; HPLC: t_{Ret}=2.48 min.

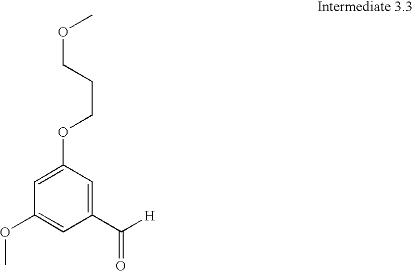

A mixture of Intermediate 3.4 (4.2 g, 18.6 mmol) and MnO₂ (10 g, 115 mmol) in toluene (100 mL) is stirred at RT for 13.5 h. Then, further MnO₂ (5 g, 57.5 mmol) is added, and the resulting mixture is further stirred at RT for 7 h. The mixture is filtered through Celite and the filtrate is concentrated in vacuo. The residue is purified by silica gel flash chromatography to give Intermediate 3.3 as a colorless oil; ES-MS: [M+H]⁺=225, HPLC: t_{Ret}=3.59 min.

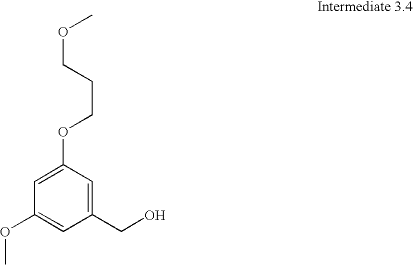

A mixture of Intermediate 3.5 (5 g, 19.7 mmol) and LAH (528 mg, 20 mmol) in THF (110 mL) is stirred under N₂ at 0° C. for 3 h. After adding H₂O, the reaction mixture is extracted with EtOAc. The combined organic phases are washed with H₂O, brine and dried (Na₂SO₄). Concentration under reduced pressure and silica gel flash chromatography give Intermediate 3.4 as colorless oil; ES-MS: [M+H]⁺=227; HPLC: t_{Ret}=2.85 min.

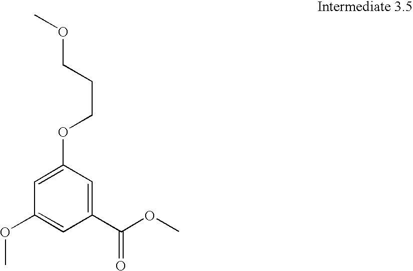

To a mixture of 3-methoxy-5-hydroxybenzoic acid methyl ester (23.2 g, 127 mmol toluene-4-sulfonic acid 3-methoxy-propyl ester (40.7 g, 167 mmol) and KI (2.23 g, 13.4 mmol) in DMF (350 mL), K₂CO₃ (53.1 g, 384 mmol) is added under N₂. After stirring at 60° C. for 17 h, the reaction mixture is supplemented with H₂O and extracted with Et₂O. The combined organic phases are washed with H₂O and dried (Na₂SO₄). Concentration under reduced pressure and silica gel flash chromatography give Intermediate 3.5 as colorless oil; ES-MS: [M+H]⁺=255, HPLC: t_{Ret}=3.80 min.

The following Examples (as racemates or preferably in essentially pure form) enlisted in Table 1 are synthesized analogously to the preparation of Examples 1-3 or as hereinbefore described or to methods described hereinbefore and hereinafter. As far as not being commercially available or available by synthesis analogous to methods or as described herein, the synthesis methods of intermediates for the preparation of compounds of Example 4-18 are described below Table 1. The asterisk (*) indicates the end of the bond at which the respective moiety is bound to the rest of the molecule falling under the following formula:

TABLE 1

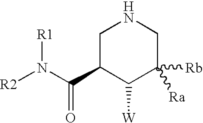

| Ex. | | | | | | Analytical |
|---|---|---|---|---|---|---|
| No. | R1— | R2— | Ra— | Rb— | W— | data |
| 4 | 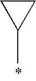 | 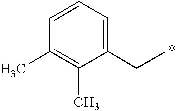 | 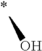 | 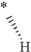 | 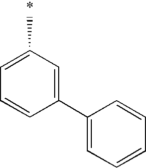 | MS: [M + 1]⁺ = 455 HPLC t_{Ret} = 3.43 |
| 5 | 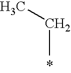 | 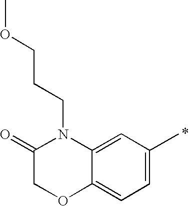 | 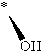 | 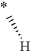 | 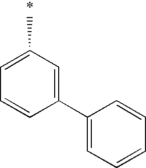 | MS: [M + 1]⁺ = 544 HPLC t_{Ret} = 3.18 |
| 6 | 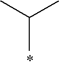 | 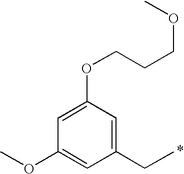 | 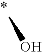 | 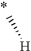 | 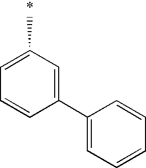 | MS: [M + 1]⁺ = 547 HPLC t_{Ret} = 3.42 |
| 7 | 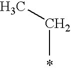 | 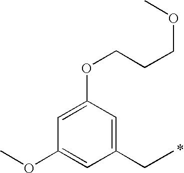 | 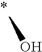 | 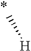 | 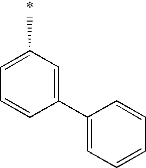 | MS: [M + 1]⁺ = 533 HPLC t_{Ret} = 3.30 |
| 8 | 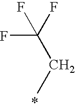 | 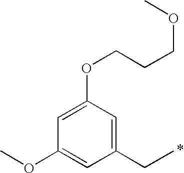 | 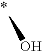 | 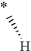 | 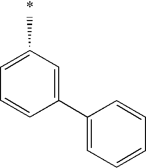 | MS: [M + 1]⁺ = 587 HPLC t_{Ret} = 3.57 |
| 9 | 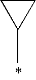 | 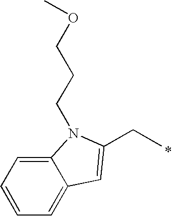 | 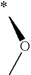 | 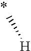 | 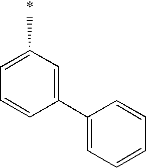 | MS: [M + 1]⁺ = 552 HPLC t_{Ret} = 3.68 |
| 10 | 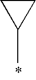 | 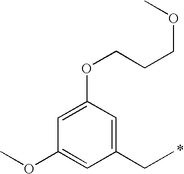 | 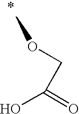 | 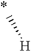 | 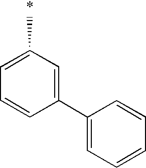 | MS: [M + 1]⁺ = 603 HPLC t_{Ret} = 3.22 |
| 11 | 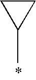 | 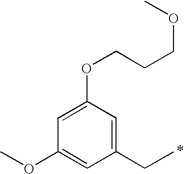 | 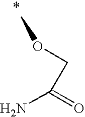 | 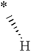 | 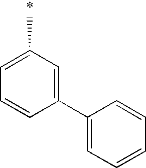 | MS: [M + 1]⁺ = 602 HPLC t_{Ret} = 3.07 |
| 12 | 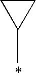 | 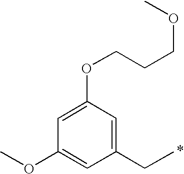 | 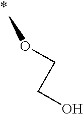 | 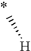 | 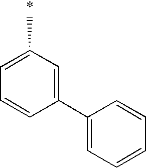 | MS: [M + 1]⁺ = 589 HPLC t_{Ret} = 3.18 |
| 13 | 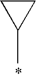 | 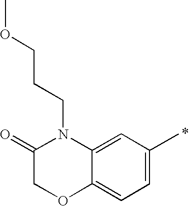 | 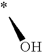 | 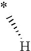 | 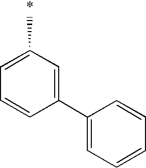 | MS: [M + 1]⁺ = 556 HPLC t_{Ret} = 2.97 |
| 14 | 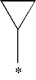 | 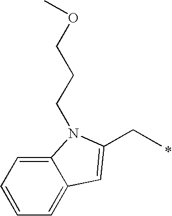 | 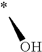 | 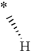 | 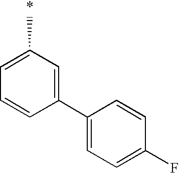 | MS: [M + 1]⁺ = 556 HPLC t_{Ret} = 3.37 |
| 15 | 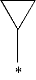 | 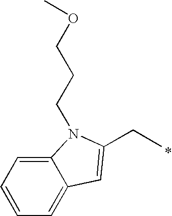 | 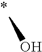 | 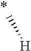 | 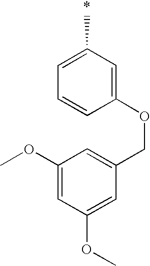 | MS: [M + 1]⁺ = 628 HPLC t_{Ret} = 3.40 |
| 16 | 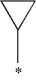 | 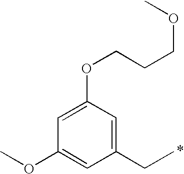 | 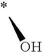 | 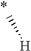 | 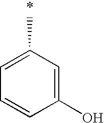 | MS: [M + 1]⁺ = 485 HPLC t_{Ret} = 2.59 |
| 17 | 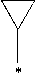 | 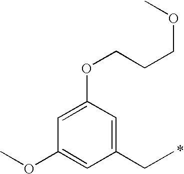 | 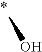 | 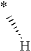 | 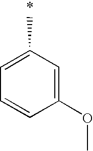 | MS: [M + 1]⁺ = 499 HPLC t_{Ret} = 2.79 |
| 18 | 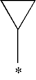 | 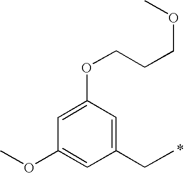 | 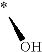 | 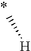 | 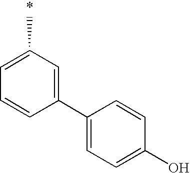 | MS: [M + 1]⁺ = 561 HPLC t_{Ret} = 2.84 |

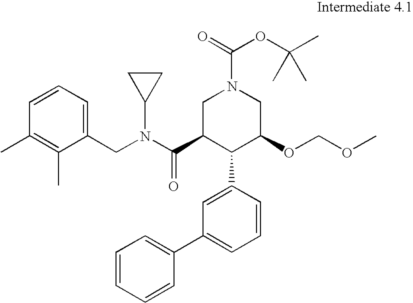

Intermediate 4.1 is synthesized by condensation of Intermediate 1.2 (500 mg, 1.13 mmol) with cyclopropyl-(2,3-dimethyl-benzyl)-amine hydrochloride salt (387 mg, 1.70 mmol) analogously to the preparation of Intermediate 1.1. White amorphous material; ES-MS: [M]+=599 HPLC: tRet=5.53 minutes.

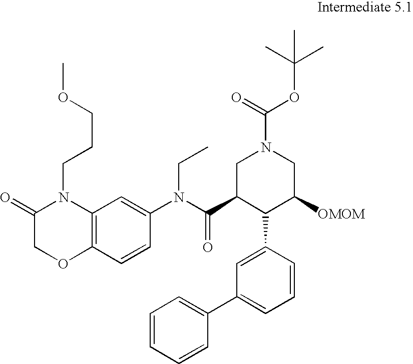

To a solution of Intermediate 1.2 (88 mg, 0.26 mmol) in DCM (2 mL), 1-chloro-N,N-2-trimethylpropaneamine (67 μL, 0.48 mmol) is added under N₂ at RT. After stirring at RT for 0.5 h, Intermediate 5.2 (66 mg, 0.22 mmol) and Et₃N (0.067 mL, 0.48 mmol) are added, and stirred under N₂ at 0° C. After stirring at RT for 1 h, saturated NaHCO₃ solution is added. The mixture is extracted with DCM and dried over Na₂SO₄. The organic layer is concentrated and purified by flash silica gel chromatography to give Intermediate 5.1.; ES-MS: M+H=688; HPLC: t_{Ret}=4.87 min.

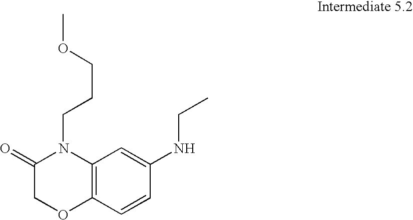

Intermediate 5.3 (1.08 g, 2.96 mmol) is treated with 4N HCl solution in 1,4-dioxane (10 mL) at RT for 2 h. the reaction mixture are concentrated under reduced pressure to give Intermediate 5.2. White powder; ES-MS: M+H=265; HPLC: t_{Ret}=2.05 min.

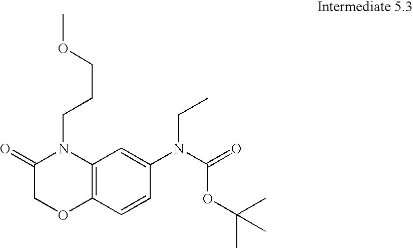

To a solution of Intermediate 5.4 (1.59 g, 4.72 mmol) in THF (20 mL), NaH (208 mg, 5.19 mmol) is added under N₂ at 0° C. After stirring at 50° C. for 0.5 h, EtI (411 μL, 5.19 mmol) is added to the mixture and stirred at 50° C. for 12 h. The reaction mixture was quenched with H₂O and extracted with EtOAc. The combined organic phases are washed with H₂O and dried over Na₂SO₄. Concentration under reduced pressure and silica gel flash chromatography give Intermediate 5.3 as colorless oil; ES-MS: M+H=309; HPLC: t_{Ret}=4.03 min.

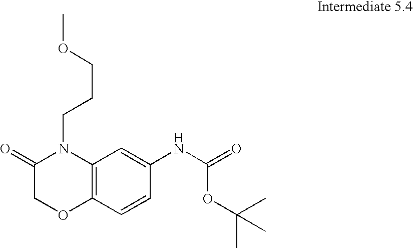

A mixture of Intermediate 5.5 (1.36 g, 5.74 mmol), Boc₂O (2.9 g, 12.6 mmol), and Et₃N (1.92 mL, 7.8 mmol) in THF (20 mL) is stirred under N₂ at RT for 2 h. After adding H₂O, the reaction mixture is extracted with EtOAc. The combined organic phases are washed with H₂O and dried (Na₂SO₄). Concentration under reduced pressure and silica gel flash chromatography give Intermediate 5.4 as colorless amorphous; ES-MS: M+H=337; HPLC: t_{Ret}=3.67 min.

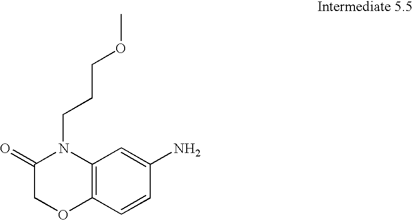

A mixture of Intermediate 5.6 (266 mg, 1 mmol) and iron powder (260 mg, 5 mmol) in EtOH (5 mL)-5N HCl (0.4 mL, 2 mmol) is stirred under N₂ at 60° C. for 3.5 h. After adding 6N KOH solution, the reaction mixture is extracted with EtOAc. The combined organic phases are dried (Na₂SO₄) and concentrated under reduced pressure to give Intermediate 5.5. Brown oil; ES-MS: [M+H]⁺=237; HPLC: t_{Ret}=1.78 min.

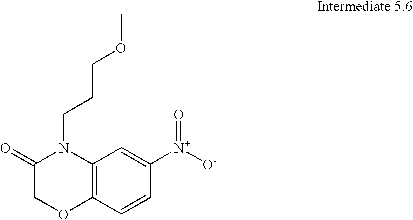

To a solution of 6-nitro-2H-1,4-benzoxazin-3(4H)-one (582 mg, 3.00 mmol) in DMF (5 mL) at 0° C., toluene-4-sulfonic acid 3-methoxy-propyl ester (1.1 g, 4.50 mmol) and KI (49.8 mg, 0.3 mmol) are added. The reaction mixture is stirred at 60° C. for 8.5 h, and water is poured in. The resulting mixture is extracted with EtOAc, and the combined organic extracts are dried (Na₂SO₄). Concentration under reduced pressure and silica gel flash chromatography give Intermediate 5.6. Yellow powder; ES-MS: [M+H]⁺=267; HPLC: t_{Ret}=3.18 min.

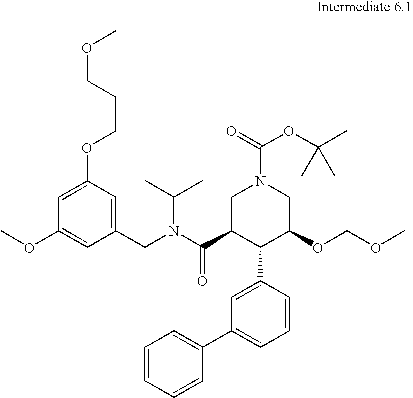

Intermediate 6.1 is synthesized by condensation of Intermediate 1.2 (70 mg, 0.16 mmol) with Intermediate 6.2 (51 mg, 0.19 mmol) analogously to the preparation of Intermediate 1.1. White amorphous material; ES-MS: [M]+=691 HPLC: tRet=5.35 minutes.

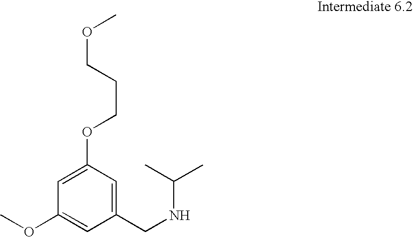

A mixture of Intermediate 3.3 (236 mg, 0.816 mmol), cyclopropylamine (0.35 mL, 4.1 mmol), and triethylamine (0.6 mL, 4.3 mmol) in THF (3 mL) is stirred at RT for 5 h. After dilution with EtOAc, the mixture is washed with water (×2) and brine and then dried (Na₂SO₄). Concentration under reduced pressure gives Intermediate 6.2. Colorless oil; ES-MS: [M+H]⁺=268; HPLC: t_{Ret}=2.43 min.

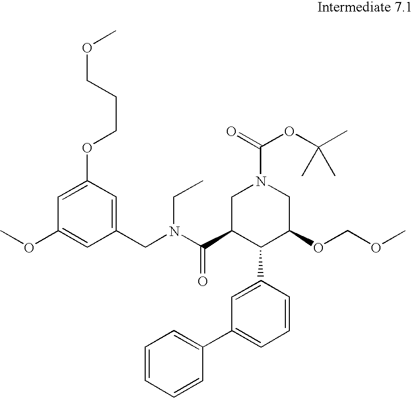

Intermediate 7.1 is synthesized by condensation of Intermediate 1.2 (70 mg, 0.16 mmol) with Intermediate 7.2 (54 mg, 0.21 mmol) analogously to the preparation of Intermediate 1.1. White amorphous material; ES-MS: [M]+=HPLC: tRet=minutes.

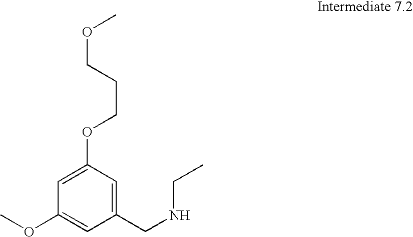

Intermediate 7.2 is synthesized by condensation of Intermediate 3.3 (217 mg, 0.75 mmol) and ethylamine (0.6 mL, 30-40% in MeOH) analogously to the preparation of Intermediate 6.2. Colorless oil; ES-MS: [M+H]⁺=254; HPLC: t_{Ret}=2.37 min.

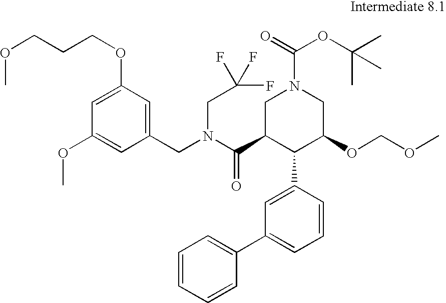

To a mixture of Intermediate 8.2 (271 mg, 0.4 mmol) and Intermediate 3.3 (116 mg, 0.4 mmol) in THF (5 mL), 1 M THF solution of NaN(TMS)₂ (0.4 mL, 0.4 mmol) is added under N₂ at 0° C. After stirring at 40 ° C. for 19 h and adding H₂O, the reaction mixture is extracted with EtOAc. The combined organic phases are dried (Na₂SO₄). Concentration under reduced pressure and RP-HPLC give Intermediate 8.1 as white amorphous material; ES-MS: [M+H]⁺=675; HPLC: t_{Ret}=5.45 min.

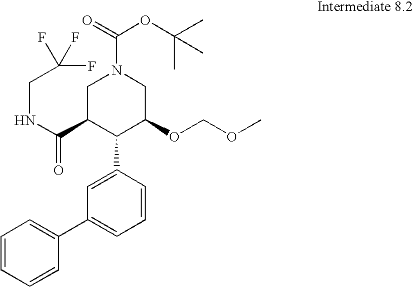

The compound of Intermediate 8.2 is synthesized by condensation of Intermediate 1.2 (176 mg, 0.4 mmol) with trifluoroethylamine (0.038 mL, 0.48 mmol) analogously to Intermediate 1.1. MS: [M+H]⁺=523; HPLC: t_{Ret}=4.59 min.

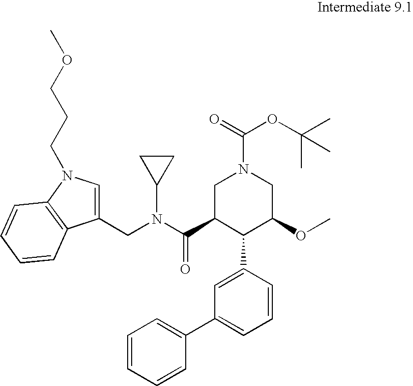

The compound of Intermediate 9.1 is synthesized by condensation of Intermediate 9.2 (170 mg, 0.41 mmol) with Intermediate 2.4 (111 mg, 0.53 mmol) analogously to Intermediate 1.1. MS: [M+H]⁺=652; HPLC: t_{Ret}=5.39 min.

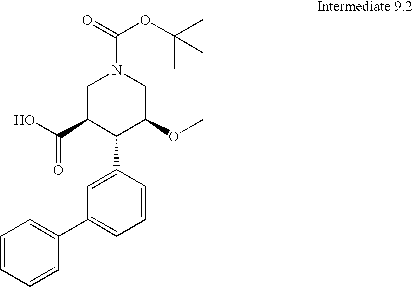

The compound of Intermediate 9.2 is synthesized by hydrolysis of Intermediate 9.3 (197 mg, 0.46 mmol) with 8N KOH (0.5 mL, 4.0 mmol) analogously to Intermediate 1.2. MS: [M+H−tBu]⁺=356; HPLC: t_{Ret}=4.26 min.

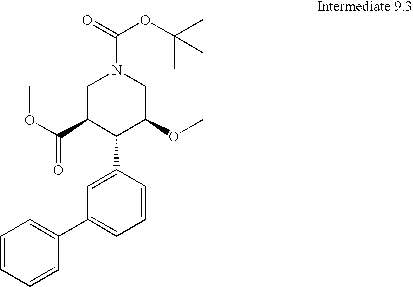

A mixture of compound of Intermediate 1.4 (200 mg, 0.49 mmol), NaH (60% oil, 23 mg, 0.59 mmol), and CH₃I (166 mg, 1.1 mmol) in DMF (2 mL) is stirred at RT for 3 h. After adding H₂O, the reaction mixture is extracted with EtOAc. The combined organic phases are washed with H₂O and dried (MgSO₄), and then concentrated under reduced pressure to give Intermediate 9.3 as colorless oil; ES-MS: [M+H]⁺=426; HPLC: t_{Ret}=4.85 min.

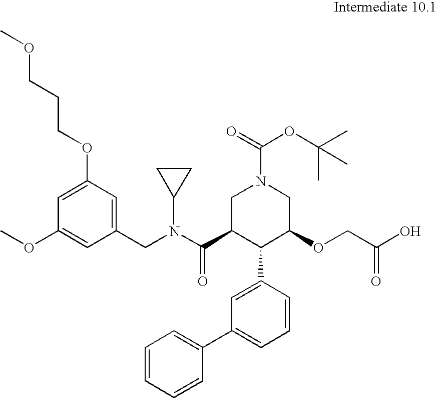

To a solution of Intermediate 10.2 (700 mg, 0.92 mmol) in CH₂Cl₂ (5 mL) is added TFA (5 mL) at room temperature. After stirring for 4 h, the reaction mixture is concentrated in reducing pressure. To a solution of the residue in CH₂Cl₂ are added Et₃N (501 uL, 3.6 mmol) and Boc₂O (392 mg, 1.8 mmol) at room temperature. After stirring for 12 h at room temperature, the reaction mixture is acidified with 1N KHSO₄ solution and extracted with CH₂Cl₂. The organic layer is washed with brine, dried over Na₂SO₄, and concentrated. Silica gel flash chromatography gives Intermediate 10.1 as white amorphous; ES-MS: M+H=703; HPLC: t_{Ret}=4.52 min.

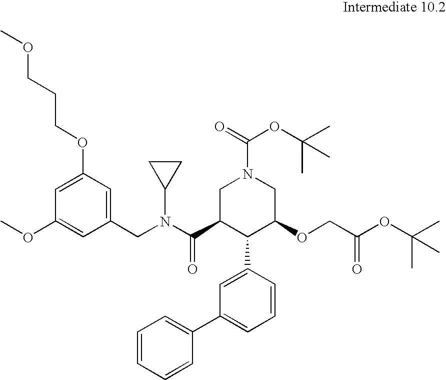

Intermediate 10.2 is synthesized by alkylation of Intermediate 10.3 (300 mg, 0.465 mmol) with t-butyl bromoacetate (138 uL, 0.93 mmol) analogously to the preparation of Intermediate 9.3. White amorphous material; ES-MS: [M+H]⁺=645; HPLC: t_{Ret}=4.59 minutes.

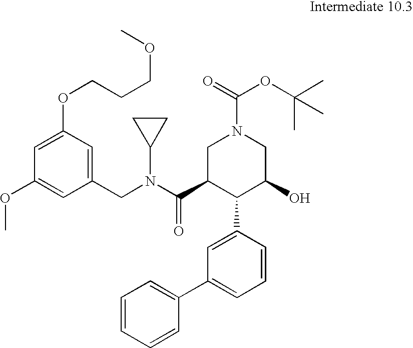

A mixture of the title compound of Example 3 (70 mg, 0.13 mmol), 1N NaOH aq. (0.15 mL, 0.15 mmol) and Boc₂O (42 mg, 0.19 mmol) in Dioxane (10 mL) is stirred at RT for 10 h. Dioxane is removed in vacuo, and H₂O and Et₂O are added to the residue. The reaction mixture is extracted with Et₂O. The combined organic phases are washed with H₂O and dried (MgSO₄) and then concentrated under reduced pressure. The resulting solution is purified by silica gel flash chromatography to give Intermediate 10.3 as colorless oil; ES-MS: [M+H]⁺=645; HPLC: t_{Ret}=7.75 min.

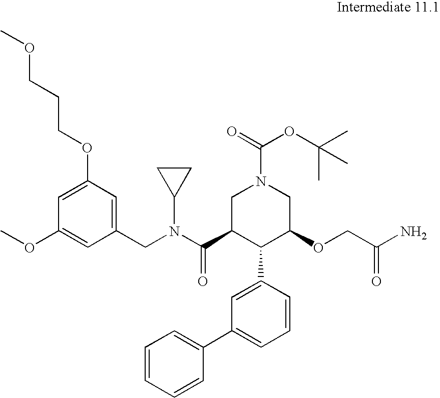

To a solution of Intermediate 10.1 (100 mg, 0.14 mmol) in THF are added Et₃N and isobutyl chloroformate (16 uL, 0.168 mmol) at 0° C. After stirring for 1h at 0° C., the reaction mixture is added aqueous ammonia (25%) at 0° C. After stirring for 1.5h at 0° C., the reaction mixture is diluted with H₂O and extracted with CH₂Cl₂. The organic layer is washed with brine, dried over Na₂SO₄, and concentrated. RP-HPLC purification gives Intermediate 11.1 as white amorphous; ES-MS: M+H=702; HPLC: t_{Ret}=4.35 min.

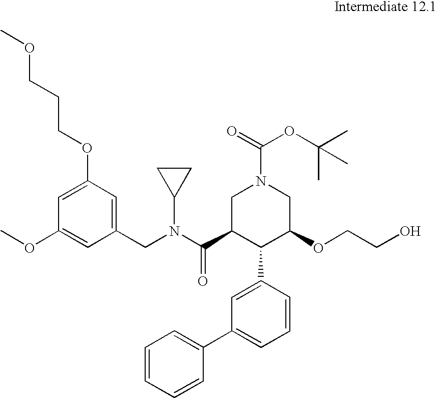

To a solution of Intermediate 10.1 (100 mg, 0.14 mmol) in THF are added Et₃N and isobutyl chloroformate (16 uL, 0.168 mmol) at 0° C. After stirring for 1 h at 0° C., the white materials are removed from the reaction mixture by filtration through celite pad, then the filtrate is concentrated in reducing pressure. To the resulted residue in THF are added NaBH₄ (27 mg, 0.7 mmol) and H₂O (0.5 mL) at 0° C. After stirring for 1 h at room temperature, the reaction mixture is diluted with H₂O and extracted with CH₂Cl₂. The organic layer is washed with brine, dried over Na₂SO₄, and concentrated. RP-HPLC purification gives Intermediate 12.1 as white amorphous; ES-MS: M+H=689; HPLC: t_{Ret}=4.60 min.

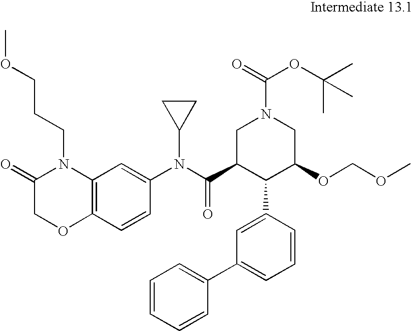

To a solution of intermediate 1.2 (98.4 mg, 0.22 mmol) in DCM (3 mL) at RT, (1-chloro-2-methyl-propenyl)-dimethyl-amine (44.8 μL,0.33 mmol) is added. After stirring at RT for 30 min, pyridine (54.8 μL, 0.678 mmol) and 6-cyclopropylamino-4-(3-methoxy-propyl)-4H-benzo[1,4]oxazin-3-one (62.4 mg, 0.226 mmol) dissolved in DCM (5 mL) is added at 0° C. The reaction mixture is stirred at RT overnight, H₂O is added and extracted with DCM, dried over Na₂SO₄, concentrated under reduced pressure and subjected to silicagel chromatography to give intermediate 13.1 as white amorphous ES-MS: M+H=700; HPLC: t_{Ret}=4.64 min

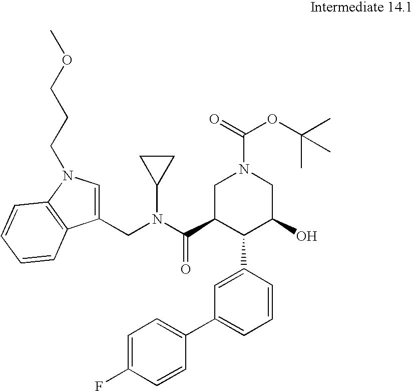

To a solution of Intermediate 14.2 (90 mg, 0.117 mmol) in THF is added TBAF (1M in THF, 0.4 mmol). After stirring for 2 h at room temperature, the resulting mixture is acidified with 1N KHSO₄ solution and extracted with CH₂Cl₂. The organic layer is washed with brine, dried over Na₂SO₄, and concentrated to give Intermediate 14.1 as white amorphous; ES-MS: M+H=656; HPLC: t_{Ret}=4.75 min.

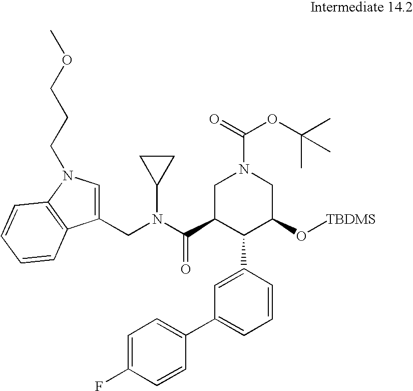

Intermediate 142 is synthesized by condensation of Intermediate 14.3 (190 mg, 0.35 mmol) with Intermediate 2.4 (90.4 mg, 0.35 mmol) analogously to the preparation of Intermediate 2.1. White amorphous material; ES-MS: [M+H]⁺=770; HPLC: t_{Ret}=5.95 minutes.

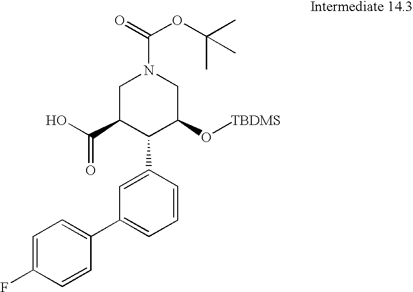

Intermediate 14.3 is synthesized by hydrolysis of Intermediate 14.4 (165 mg, 0.35 mmol) analogously to the preparation of Intermediate 2.2. White amorphous material; ES-MS: [M+H]⁺=474; HPLC: t_{Ret}=5.49 minutes.

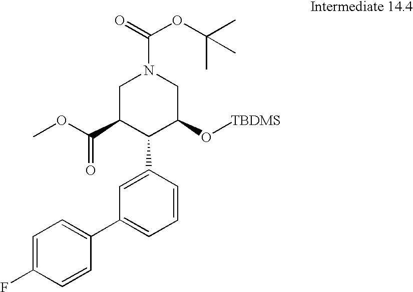

Intermediate 14.4 is synthesized by hydrolysis of Intermediate 14.5 (150 mg, 0.35 mmol) analogously to the preparation of Intermediate 2.3. White amorphous material; ES-MS: [M+H]⁺=488; HPLC: t_{Ret}=6.30 minutes.

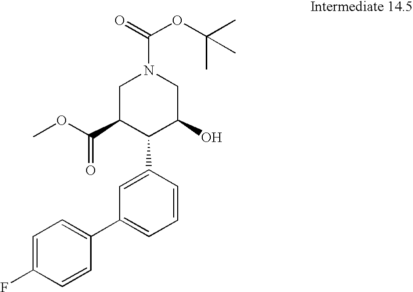

Intermediate 14.5 is synthesized by cross coupling of Intermediate 14.6 (380 mg, 0.79 mmol) and 4-fluorophenylboronic acid (165 mg, 1.18 mmol) analogously to the preparation of Intermediate 1.9. White amorphous material; ES-MS: [M−^{t}Bu+H]⁺=374; HPLC: t_{Ret}=4.30 minutes.

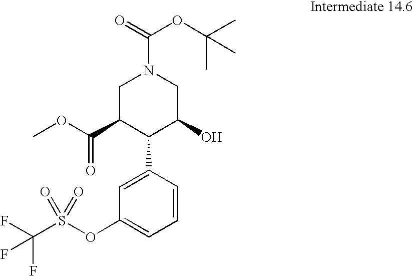

Intermediate 14.6 is synthesized by sulfonylation of Intermediate 14.7 (350 mg, 0.13 mmol) analogously to the preparation of Intermediate 27.5. White amorphous material; ES-MS: [M−^{t}Bu+H]⁺=428; HPLC: t_{Ret}=4.22 minutes.

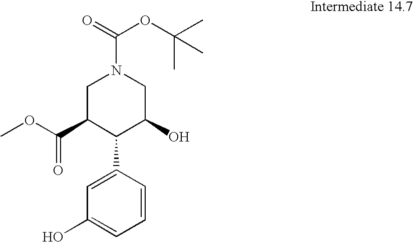

Intermediate 14.7 is synthesized by deprotection then protection of Intermediate 14.8 (500 mg, 0.1.26 mmol) analogously to the preparation of Example 2. White amorphous material; ES-MS: [M−^{t}Bu+H]⁺=296; HPLC: t_{Ret}=3.22 minutes.

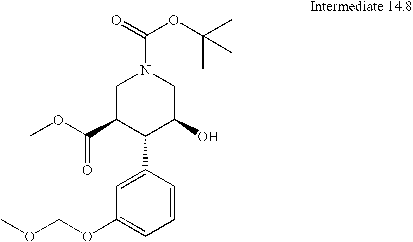

Intermediate 14.8 is synthesized by reduction of Intermediate 14.9 (1.4 g, 3.56 mmol) analogously to the preparation of Intermediate 1.5. White amorphous material; ES-MS: [M+H]⁺=396; HPLC: t_{Ret}=3.60 minutes.

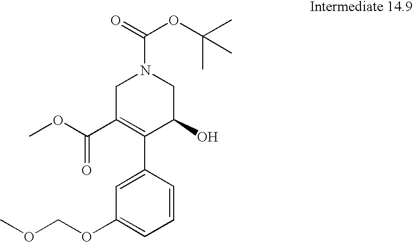

Intermediate 14.9 is synthesized by isomerization of Intermediate 14.10 (4.2 g, 10.7 mmol) analogously to the preparation of Intermediate 1.6. White amorphous material; ES-MS: [M−^{t}Bu+H]⁺=338; HPLC: t_{Ret}=3.65 minutes.

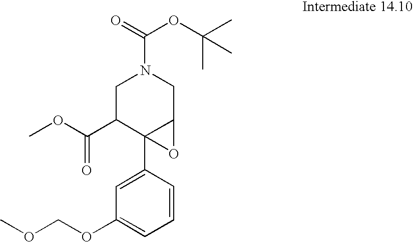

Intermediate 14.10 is synthesized by oxidation of Intermediate 14.11 (3.3 g, 8.7 mmol) analogously to the preparation of Intermediate 1.7. White amorphous material; ES-MS: [M+H]⁺=338; HPLC: t_{Ret}=4.12 minutes.

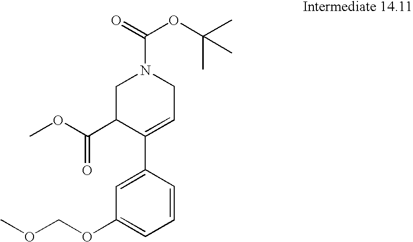

Intermediate 14.11 is synthesized by isomerization of Intermediate 14.12 (4.1 g, 10.8 mmol) analogously to the preparation of Intermediate 1.8. White amorphous material; ES-MS: [M+H]⁺=378; HPLC: t_{Ret}=4.27 minutes.

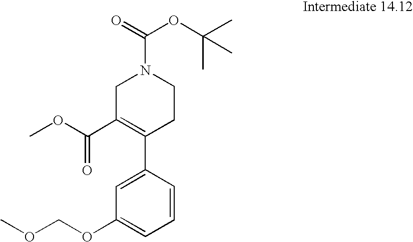

Intermediate 14.12 is synthesized by protection of Intermediate 14.13 (5 g, 15 mmol) analogously to the preparation of Intermediate 1.3. White amorphous material; ES-MS: [M+H]⁺=378; HPLC: t_{Ret}=4.39 minutes.

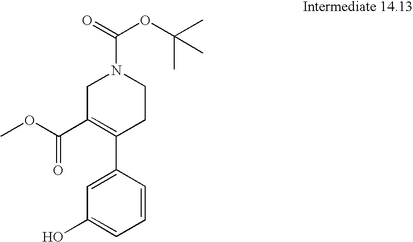

Intermediate 14.12 is synthesized by cross coupling reaction of 3-phenylboronic acid (8.5 g, 61.7 mmol) analogously to the preparation of Intermediate 1.9. White amorphous material; ES-MS: [M+H]⁺=334; HPLC: t_{Ret}=3.79 minutes.

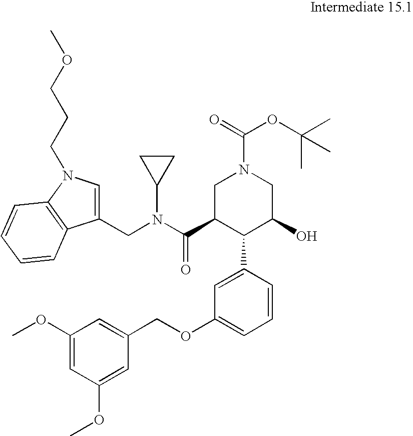

Intermediate 15.1 is synthesized by deprotection of Intermediate 15.2 analogously to the preparation of example 2. White amorphous material; ES-MS: [M+H]⁺=728; HPLC: t_{Ret}=4.75 minutes.

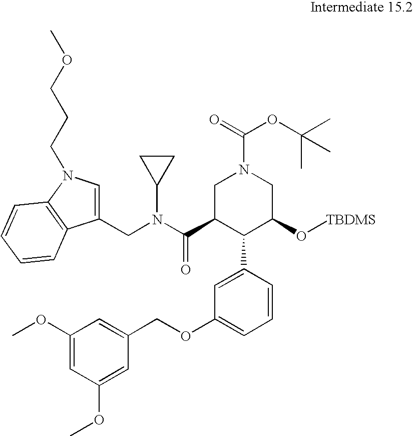

Intermediate 15.2 is synthesized by condensation reaction Intermediate 15.3 analogously to the preparation of Intermediate 1.1. White amorphous material; ES-MS: [M]⁺=842; HPLC: t_{Ret}=5.85 minutes.

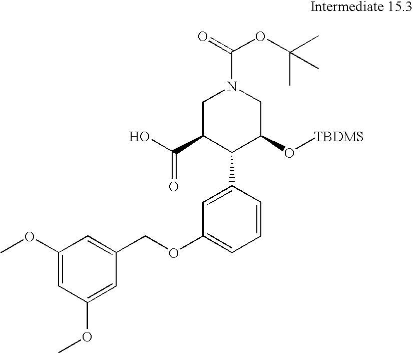

Intermediate 15.3 is synthesized by hydrolysis of Intermediate 15.4 analogously to the preparation of Intermediate 2.2. White amorphous material; ES-MS: [M+H]⁺=602; HPLC: t_{Ret}=5.37 minutes.

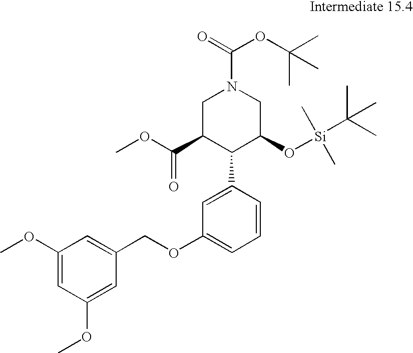

Intermediate 15.4 is synthesized by protection of Intermediate 15.5 (450 mg, 0.897 mmol) analogously to the preparation of Intermediate 2.3. White amorphous material; ES-MS: [M+H]⁺=616; HPLC: t_{Ret}=6.17 minutes.

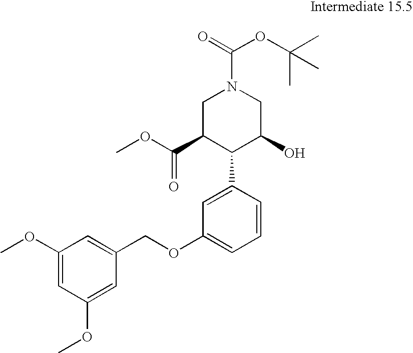

To a solution of Intermediate 14.7 in DMF are added k₂CO₃ (189 mg, 1.37 mmol) and 3,5-dimethoxybenzyl bromide (316 mg, 1.37 mmol). After stirring at 40° C. for 17 h, the reaction mixture is diluted with H₂O and extracted with EtOAc. The organic layer is washed with brine, dried over Na₂SO₄, and concentrated. RP-HPLC purification gives Intermediate 15.5 as white amorphous; ES-MS: M+H=503; HPLC: t_{Ret}=4.34 min.

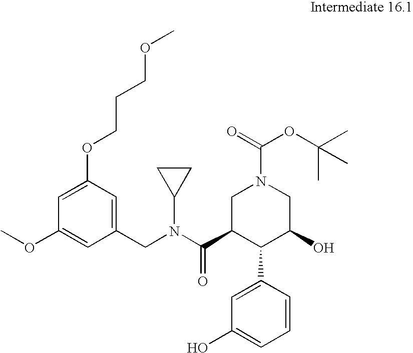

A mixture of Intermediate 16.2 (500 mg, 0.637 mmol) and 2N HCl in MeOH are stirred for 1.5 h at room temperature. Concentration in reducing pressure gives the crude. To the crude in CH₂Cl₂ (5 mL) are added Et₃N and Boc₂O. After stirring for 18 h, the reaction mixture is diluted with H₂O and extracted with CH₂Cl₂. The combined organic layer is washed with brine, dried over Na₂SO₄. Concentrated in reduced pressure and silica gel column chromatography gives Intermediate 16.1 as white amorphous; ES-MS: M+H=585; HPLC: t_{Ret}=3.72 min.

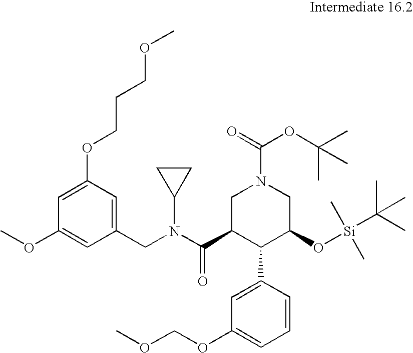

Intermediate. 16.2 is synthesized by condensation of Intermediate 16.3 (480 mg, 0.97 mmol) with Intermediate 3.2 (308 mg, 1.16 mmol) analogously to the preparation of Intermediate 1.1. White amorphous material; ES-MS: [M=743; HPLC: t_{Ret}=5.57 minutes.

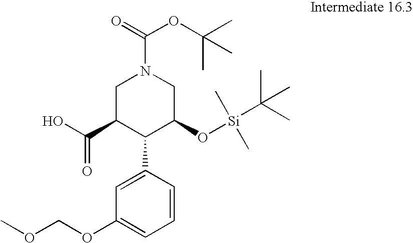

To a solution of Intermediate 16.4 (700 mg, 1.84 mmol) in DMF are added imidazole (376 mg, 5.52 mmol) and TBDMSCl (832 mg, 5.52 mmol) at room temperature. After stirring for 7 days, the reaction mixture is diluted with 5% aqueous KHSO₄ and extracted with Et₂O. The organic layer is washed H₂O with brine, dried over Na₂SO₄. Concentrated in reduced pressure gives the crude.

To the crude in THF/MeOH/H₂O is added LiOH H₂O (1.54 mg, 18.4 mmol) at room temperature. After stirring for 3 days, the reaction mixture is diluted with 5% aqueous KHSO₄ and extracted with EtOAc. The organic layer is washed H₂O with brine, dried over Na₂SO₄. Concentrated in reduced and silica gel column chromatography give Intermediate 16.3 as white amorphous; ES-MS: [M−^{t}Bu+H]⁺=440; HPLC: t_{Ret}=4.99 min.

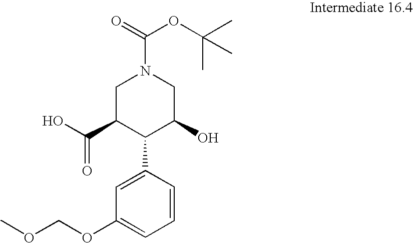

A mixture of Intermediate 14.9 (3 g, 7.5 mmol) in toluene (60 mL) is cooled to −40° C. To the cooled solution, dropwise over 10 min 65% Red-Al (3.6 mL, 11.3 mmol) in toluene is added while maintaining the internal temperature below −45° C. After stirring for 3 h at −40° C., the reaction mixture is quenched by 5% aqueous KHSO₄ and with Et₂O (30 mL, 2×). The combined organic phase are washed with H₂O, brine and dried (MgSO₄), then concentrated under reduced pressure to give the crude.

A mixture of the crude and NaOMe in MeOH is refluxed at 80° C. for 5 h. The solvent is remove in reducing pressure, and the residue is suspended in DCM and 5% aqueous KHSO₄. The organic layer is washed with H₂O and brine, dried over MgSO₄ and subjected to silica gel flash chromatography to give Intermediate 16.4 and corresponding ester as a white amorphous materials; the acid; ES-MS: [M+H−tBu]⁺=326; HPLC: t_{Ret}=3.13 min; the ester; ES-MS: [M+H−tBu]⁺=340; HPLC: t_{Ret}=3.70 min.

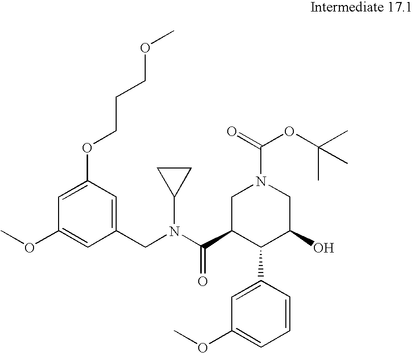

Intermediate 17.1 is synthesized by alkylation of Intermediate 16.1 (90 mg, 0.15 mmol) analogously to the preparation of Intermediate 15.5. White amorphous material; ES-MS: [M+H]⁺=599; HPLC: t_{Ret}=4.05 minutes.

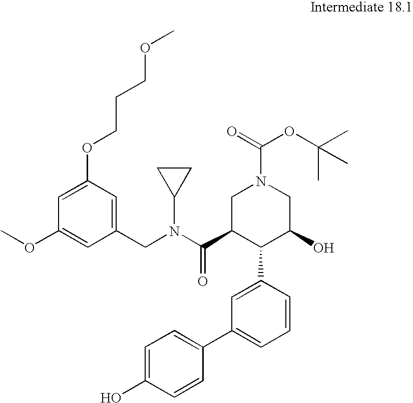

Intermediate 18.1 is synthesized by cross coupling reaction of Intermediate 18.2 (290 mg, 0.41 mmol) and 4-phenylboronic acid (110 mg, 0.8 mmol) analogously to the preparation of Intermediate 1.9. White amorphous material; ES-MS: [M+H]⁺=661; HPLC: t_{Ret}=3.97 minutes.

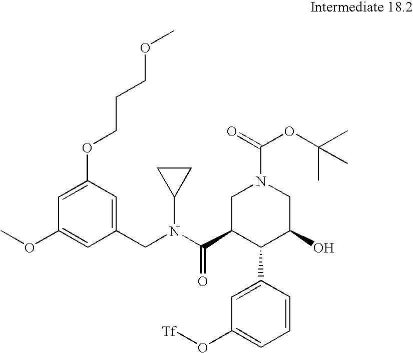

Intermediate 18.2 is synthesized by sulfonylation of Intermediate 18.3 (250 mg, 0.43 mmol) analogously to the preparation of Intermediate 27.5. White amorphous material; ES-MS: [M+H]⁺=717; HPLC: t_{Ret}=4.59 minutes.

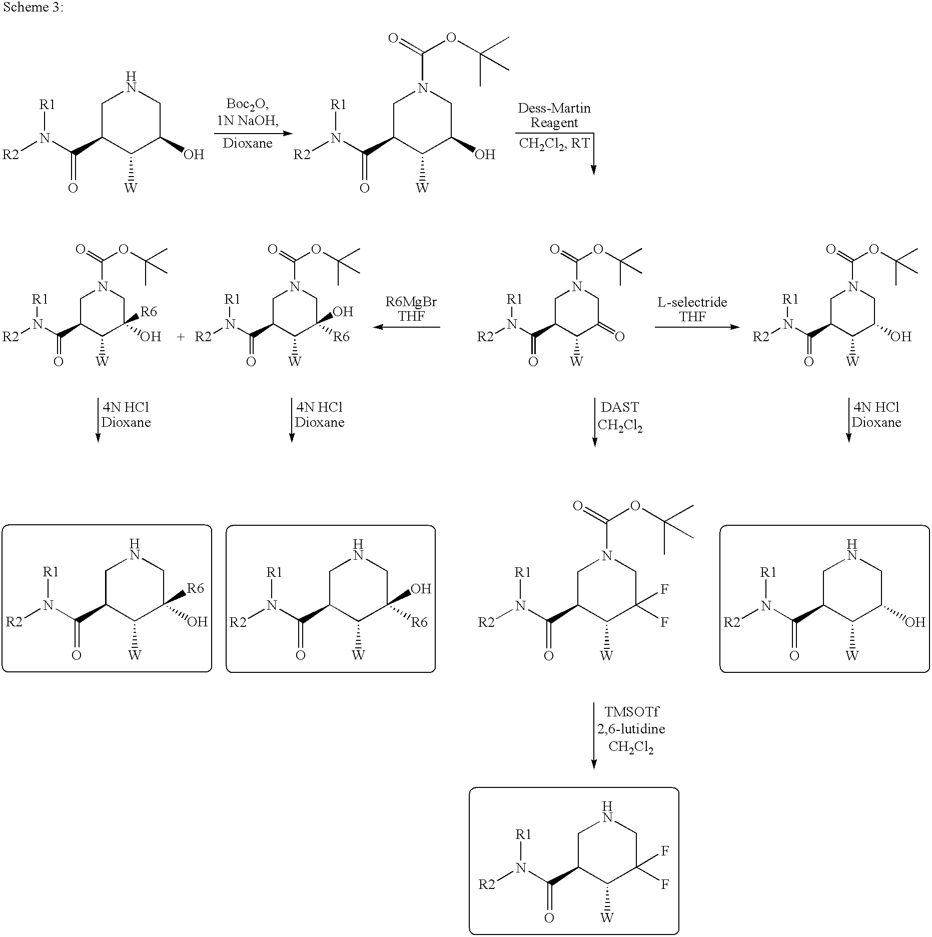

R1, R2, R6 and W are as defined for the corresponding moieties in formula I in the following Examples 19 to 23:

### Example 19

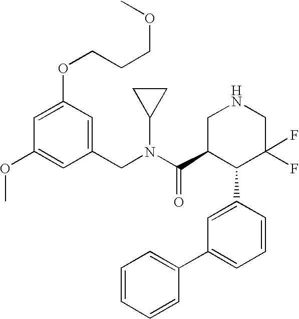

The compound of Example 19 is synthesized by deprotection of Intermediate 191 (79 mg, 0.12 mmol) with TMSOTf (0.02 mL, 0.12 mmol) and 2,6-lutidine (0.024 mL, 0.24 mmol) analogously to Example 2. MS: [M+H]⁺=565; HPLC: t_{Ret}=3.65 min.

### Example 20

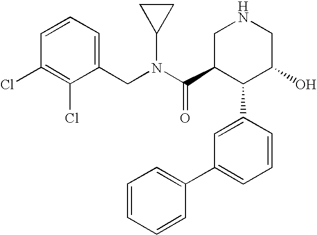

The compound of Example 20 is synthesized by deprotection of Intermediate 20.1 (61 mg, 0.10 mmol) analogously to Example 1. MS: [M+H]⁺=495; HPLC: t_{Ret}=3.77 min.

### Example 21

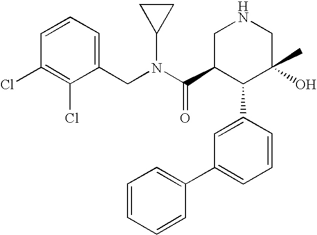

The compound of Example 21 is synthesized by deprotection of Intermediate 21.1 (18 mg, 0.03 mmol) analogously to Example 1. MS: [M+H]⁺=509; HPLC: t_{Ret}=3.84 min.

### Example 22

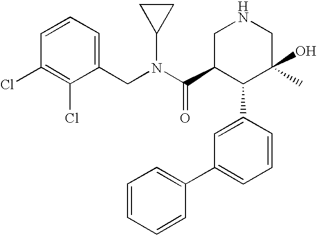

The compound of Example 22 is synthesized by deprotection of Intermediate 22.1 (18 mg, 0.03 mmol) analogously to Example 1. MS: [M+H]⁺=509; HPLC: t_{Ret}=3.72 min.

### Example 23

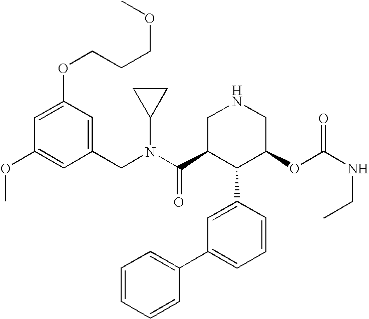

The compound of Example 23 is synthesized by deprotection of Intermediate 23.1 (80 mg, 0.11 mmol) analogously to Example 1. MS: [M+H]⁺=616; HPLC: t_{Ret}=3.65 min.

The starting materials for Examples 19 to 23 are prepared as described in the following:

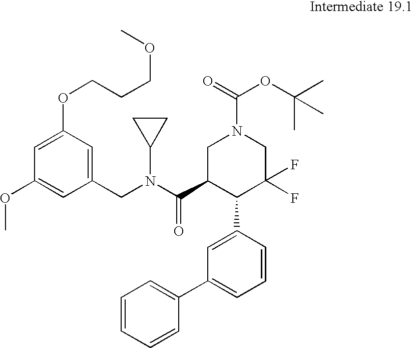

To a solution of compound of Intermediate 19.2 (80 mg, 0.12 mmol) in DCM (10 mL); DAST (0.035 mL, 0.261 mmol) is slowly added at 0° C. After this, the reaction mixture is stirred for 2 h at 0° C. and for 2 h at RT, then the reaction is quenched by the addition of sat. NaHCO₃ aq. at 0° C. The resulting mixture is extracted with DCM. The combined organic phases are washed with H₂O and dried (MgSO₄) and then concentrated under reduced pressure. The resulting solution is purified by silica gel flash chromatography to give Intermediate 19.1 as yellow oil; ES-MS: [M+H]⁺=665; HPLC: t_{Ret}=5.42 min.

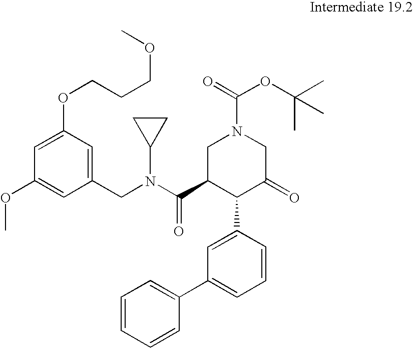

A mixture of Intermediate 10.3 (61 mg, 0.10 mmol) and Dess-Martin periodinane (60 mg, 0.14 mmol) in DCM (15 mL) is stirred at RT. After stirring for 3 h at RT, the suspension is filtrated through a pad of Celite. The filtrate is concentrated under reduced pressure. The resulting solution is purified by silica gel flash chromatography to give Intermediate 19.2 as colorless amorphous material; ES-MS: [M+H]⁺=643; HPLC: t_{Ret}=5.07 min.

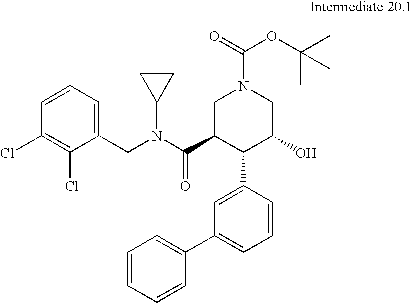

To a solution of Intermediate 20.2 (80 mg, 0.14 mmol) in THF (15 mL), a solution of 1.0M L-Selectride in THF (0.14 mL, 0.14 mmol) is slowly added at -78° C. After stirring for 30 min at −78° C., the reaction is quenched by the addition of sat. NaHCO₃ aq. at −78° C. The resulting mixture is extracted with Et₂O. The combined organic phases are washed with H₂O and dried (MgSO₄) and then concentrated under reduced pressure. The resulting solution is purified by silica gel flash chromatography to give Intermediate 20.1 as colorless amorphous material; ES-MS: [M+H⁺=595; HPLC: t_{Ret}=5.39 min.

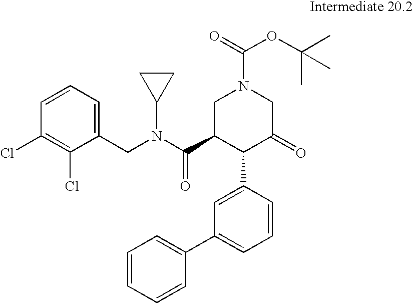

Intermediate 20.2 is synthesized by protection and oxidation of the title compound of Example 1 (250 mg, 0.50 mmol) analogously to Intermediates 19.2. MS: [M+H]⁺=596; HPLC: t_{Ret}=5.64 min.

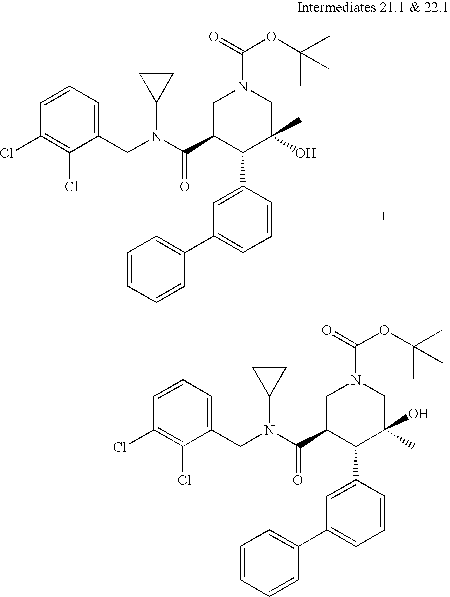

To a solution of Intermediate 20.2 (80 mg, 0.14 mmol) in THF (15 mL), a solution of 1.0M MeMgBr in THF (0.14 mL, 0.14 mmol) is slowly added at 0° C. After stirring for 10 h at RT, the reaction is quenched by the addition of sat. NaHCO₃ aq. at RT. The resulting mixture is extracted with Et₂O. The combined organic phases are washed with H₂O and dried (MgSO₄) and then concentrated under reduced pressure. The resulting solution is purified by HPLC to give Intermediate 21.1 as colorless oil ES-MS: [M+H]⁺=609; HPLC: t_{Ret}=5.35 min.) and Intermediate 22.1 as colorless oil ES-MS: [M+H]⁺=609; HPLC: t_{Ret}=5.55 min.)

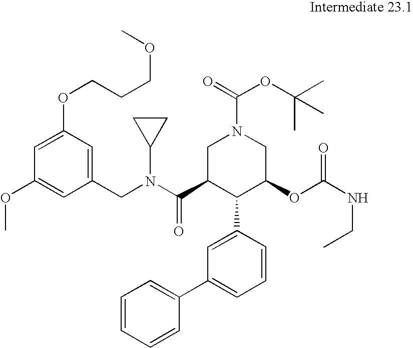

To a solution of Intermediate 10.3 (80 mg, 0.12 mmol) and ethyl isocyanate (0.03 mL, 0.37 mmol) in THF (15 mL), NaH (10.0 mg, 0.24 mmol) is added at 0° C. After stirring for 26 h at RT, the reaction is quenched by the addition of sat. NH₄Cl aq. 0° C. The resulting mixture is extracted with Et₂O. The combined organic phases are washed with H₂O and dried (MgSO₄) and then concentrated under reduced pressure. The resulting solution is purified by silica gel flash chromatography to give Intermediate 23.1 as colorless oil; ES-MS: [M+H]⁺=716; HPLC: t_{Ret}=5.03 min.

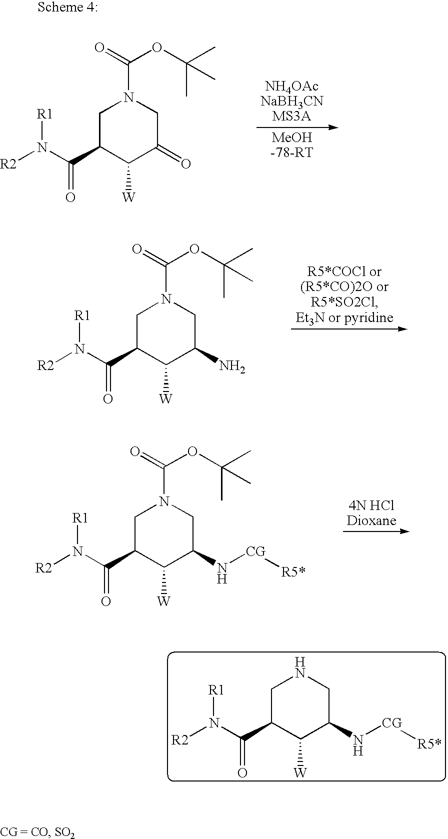

R1, R2 and W are as defined for the corresponding moieties in formula I, R5* is a moiety bound via connecting group CG which together with CG forms a corresponding moiety R5 in the following Examples 24 to 26:

### Example 24

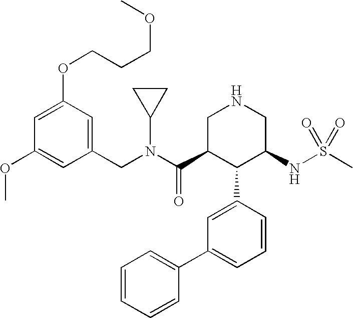

The compound of Example 24 is synthesized by deprotection of Intermediate 24.1 (43 mg, 0.06 mmol) with 4N HCl solution in Dioxane (3 mL,) analogously to Example 1. MS: [M+H]⁺=622; HPLC: t_{Ret}=3.52 min.

### Example 25

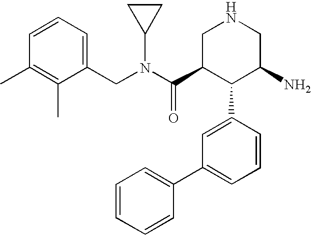

The compound of Example 25 is synthesized by deprotection of Intermediate 25.1 (46 mg, 0.08 mmol) analogously to Example 1. MS: [M+H]⁺=454; HPLC: t_{Ret}=3.17 min.

### Example 26

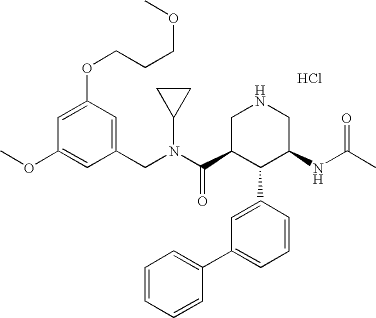

Example 26 is synthesized by deprotection of Intermediate 26.1 (21 mg, 0.031 mmol) analogously to Example 1 using 4N HCl. Colorless solidl; ES-MS: [M]+=586; HPLC: tRet=3.42 min.

The starting materials for Examples 24 and 26 are prepared as described in the following:

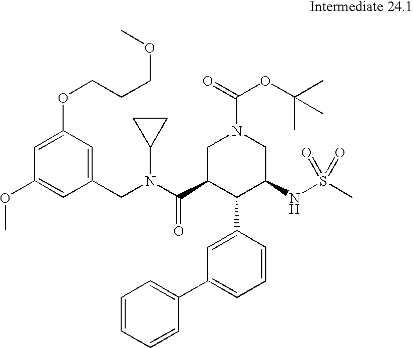

To a mixture of compound of Intermediate 24.2 (80 mg, 0.12 mmol) and NEt₃ (0.02 mL, 0.11 mmol) in DCM (5 mL), MsCl (0.01 mL, 0.09 mmol) is added at −78° C. After stirring for 3 h at RT, the suspension is diluted with DCM and H₂O. The resulting mixture is extracted with DCM. The combined organic phases are washed with H₂O and dried (MgSO₄) and then concentrated under reduced pressure. The resulting solution is purified by silica gel flash chromatography to give Intermediate 24.1 as colorless oil; ES-MS: [M+H]⁺=722; HPLC: t_{Ret}=4.87 min.

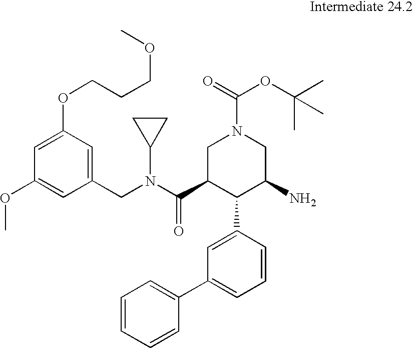

To a mixture of compound of Intermediate 19.2 (500 mg, 0.78 mmol), AcONH₄ (600 mg, 0.78 mmol), and Molecular sieves 3A (2 g) in MeOH (30 mL), NaBH₃CN (50 mg, 0.78 mmol) is added at −78° C. for 10 min. After stirring for 24 h at RT, the mixture is filtered through a pad of Celite which is further washed with DCM. The filtrate is concentrated and diluted with DCM and saturated NaHCO₃ solution. After extraction with DCM, the organic phase is dried (MgSO₄) and then concentrated under reduced pressure. The resulting solution is purified by HPLC to give Intermediate 24.2 as colorless oil; ES-MS: [M+H]⁺=644; HPLC: t_{Ret}=3.97 min.

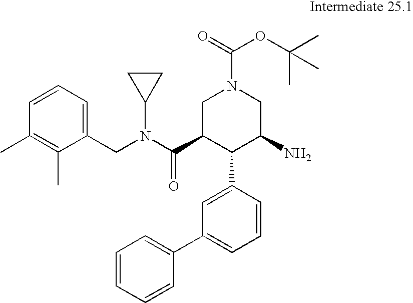

The compound of intermediate 25.1 is synthesized by reductive amination of Intermediate 25.2 (200 mg, 0.36 mmol) analogously to Intermediate 24.2. ES-MS: [M+H]⁺=554; HPLC: t_{Ret}=4.07 min.

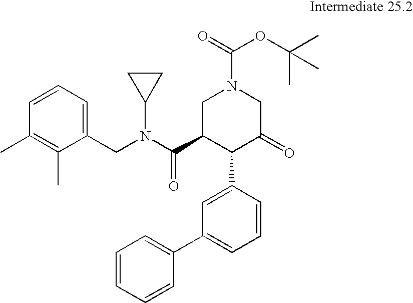

The compound of Intermediate 25.2 is synthesized by protection and oxidation of Example 4 (350 mg, 0.63 mmol) analogously to Intermediates 19.2 and 10.3. ES-MS: [M+H]⁺=553; HPLC: t_{Ret}=5.34 min.

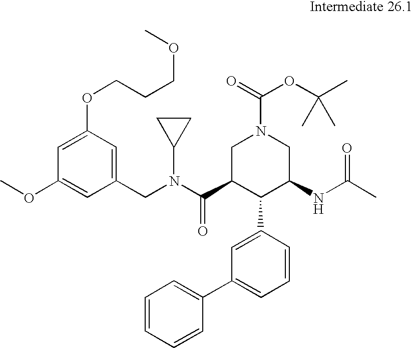

To a mixture of compound of Intermediate 24.2 (80 mg, 0.12 mmol) and pyridine (0.012 ml, 0.15 mmol) in DCM, AcCl (0.009 ml, 0.124 mmol) is added at −78° C. After stirring for 3 h at RT, the mixture is diluted with H₂O and DCM. After extraction with DCM, the organic phase is dried (MgSO₄) and then concentrated under reduced pressure. The resulting solution is purified by silica gel column chromatography to give Intermediate 26.1 as colorless oil; ES-MS: [M]+=686; HPLC: tRet =4.67 min.

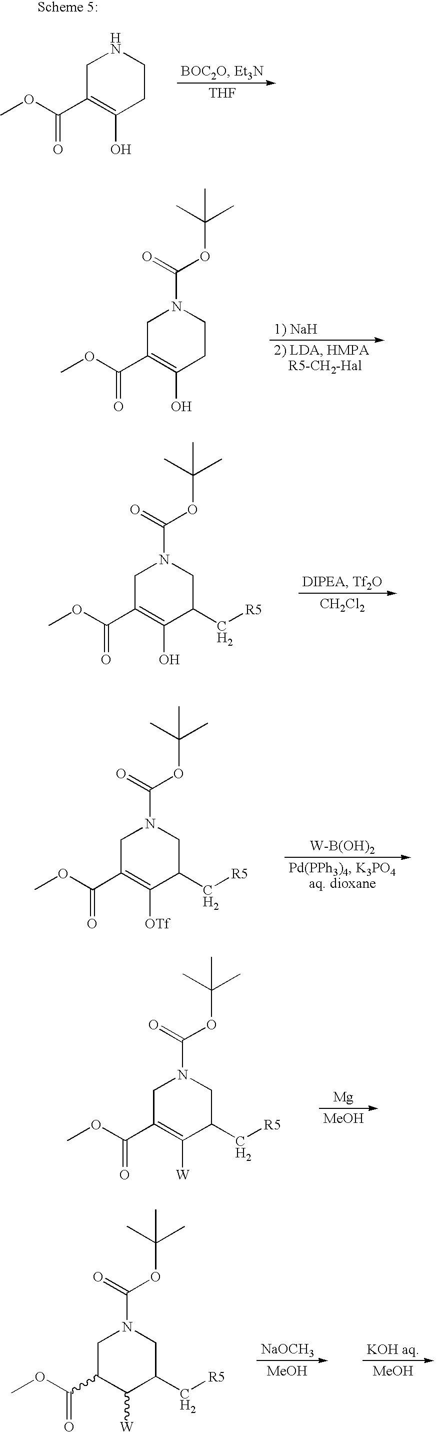

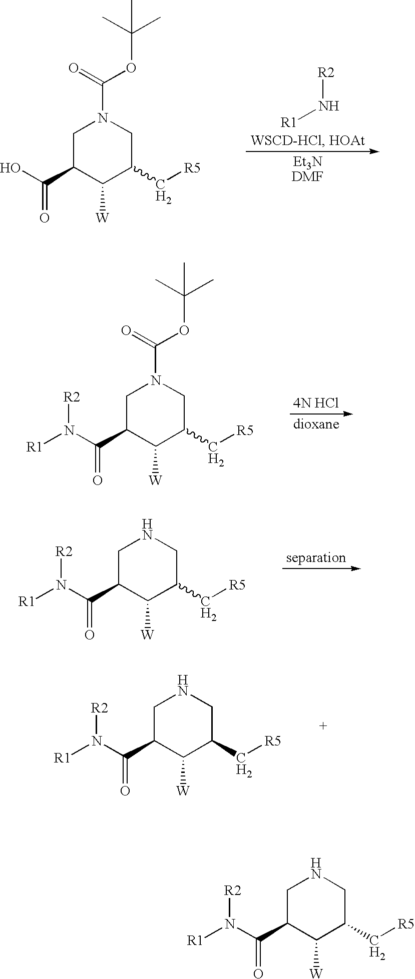

W, R1 and R2 are as defined for the corresponding moieties in formula I in the following Examples 27 to 37:

### Example 27

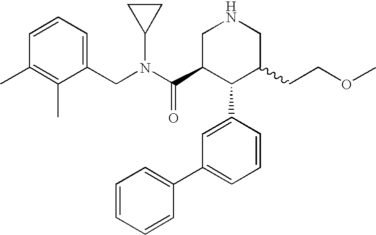

A mixture of Intermediate 27.1 (12.1 mg, 0.02 mmol) in 4N HCl-dioxane (3 mL) is stirred at RT for 2.5 h. After concentration, the resulting residue is purified by RP-HPLC to give Example 27 as a colorless oil; ES-MS: [M+H]⁺=497, HPLC: t_{Ret}=3.90 min.

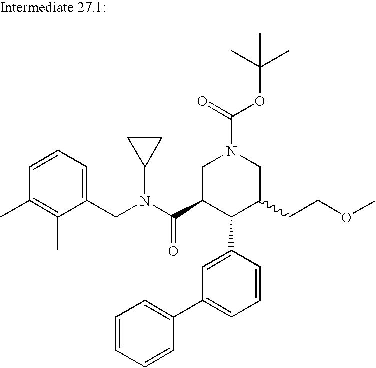

A mixture of compound of Intermediate 27.2 (44.5 mg, 0.098 mmol), cyclopropyl-(2,3-dimethyl-benzyl)-amine (25.6 mg, 0.121 mmol), EDC (27.7 mg, 0.121 mmol), Et₃N (0.0338 ml, 0.242 mmol) and HOAt (16.46 mg, 0.121 mmol) in DMF (5 mL) is stirred under N₂ at RT for 10 h and then stirred at 70° C. overnight. After adding H₂O, the reaction mixture is extracted with EtOAc. The combined organic phases are washed with H₂O and brine and dried (Na₂SO₄) and then concentrated under reduced pressure. The resulting solution is purified by silica gel flash chromatography to give Intermediate 27.1 as a colorless oil; ES-MS: [M+H]⁺=597, HPLC: t_{Ret}=5.74 min.

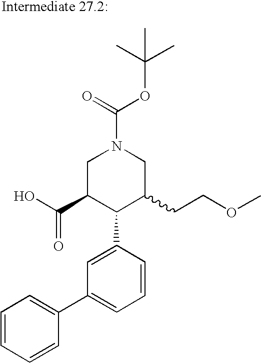

To a solution of Intermediate 27.3 (55.5 mg, 0.12 mmol) in MeOH (5 mL) under N₂, NaOCH₃ (25.8 mg, 0.47 mmol) is added at RT. The resulting mixture is stirred at 65° C. overnight. After addition of 1N KOH (1 mL), the resulting solution is refluxed for 3 h. After cooling down to 0° C., 1N HCl is added to the reaction mixture and the resulting mixture is extracted with CH₂Cl₂. The combined organic phases are dried (Na₂SO₄) and then concentrated under reduced pressure to give Intermediate 27.2 as a colorless oil; ES-MS: [M+H−tBu]⁺=384, HPLC: t_{Ret}=4.47 min.

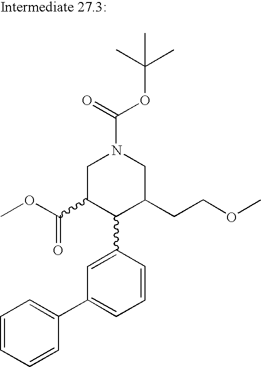

A mixture of Intermediate 27.4 (61.1 mg, 0.135 mmol) and Mg (57 mg, 2.34 mmol) in MeOH (5 mL) under N₂ is stirred at RT overnight. After evaporation, NH₄Cl is added to the reaction mixture and the resulting mixture is extracted with CH₂Cl₂. The combined organic phases are dried (Na₂SO₄) and then concentrated under reduced pressure to give Intermediate 27.3 as a colorless oil; ES-MS: [M+H−tBu]⁺=398, HPLC: t_{Ret}=5.12 min.

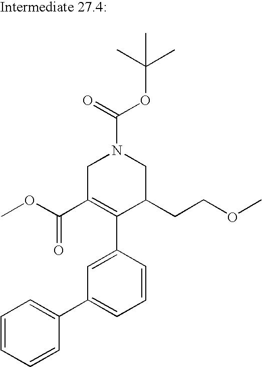

To a solution of Intermediate 27.5 (138.7 mg, 0.31 mmol) and 3-biphenylboronic acid (117.4 mg, 0.592 mmol) in dioxane (5 mL) under N₂, Pd(PPh₃)₄ (57.1 mg, 0.049 mmol) and K₃PO₄ (158 mg, 0.744 mmol) are added at RT. The resulting mixture is stirred at 80° C. overnight. After adding H₂O, reaction mixture is extracted with EtOAc. The combined organic phases are washed with brine and dried (Na₂SO₄) and then concentrated under reduced pressure. The resulting residue is purified by silica gel flash chromatography to give Intermediate 27.4 as a colorless oil; ES-MS: [M+H−tBu]⁺=396, HPLC: t_{Ret}=5.18 min.

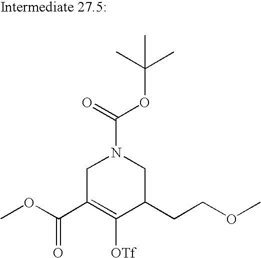

To a solution of Intermediate 27.6 (99.6 mg, 0.31 mmol) and iPr₂NEt (0.207 ml, 1.2 mmol) in CH₂Cl₂ (5 mL) under N₂, Tf₂O (0.18 mmol, 1.089 mmol) is added at 0° C. The resulting mixture is stirred at 0° C. overnight. After adding NaHCO₃ aq., the reaction mixture is extracted with EtOAc. The combined organic phases are dried (Na₂SO₄) and concentrated under reduced pressure. The resulting residue is purified by silica gel flash chromatography to give Intermediate 27.5 as a colorless oil; ES-MS: [M+H−BOC]⁺=348, HPLC: t_{Ret}=4.68 min.

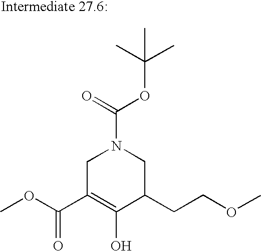

To a solution of Intermediate 27.7 (552 mg, 2.0 mmol) in THF (5 mL), NaH (81 mg, 2.0 mmol) is added at 0° C. After stirring at 0° C. for 15 min, 2M LDA (1.0 mL, 2.0 mmol) and HMPA (0.7 mL, 4.0 mmol) are added to the reaction mixture. After stirring at 0° C. for 15 min, 2-chloroethyl methyl ether is added to the resulting solution. The reaction mixture is stirred at RT overnight. After addition of NH₄Cl aq., reaction mixture is extracted with EtOAc. The combined organic phases are washed with brine and dried (Na₂SO₄), concentrated under reduced pressure. The resulting residue is purified by silica gel flash chromatography to give Intermediate 27.6 as a colorless oil; ES-MS: [M+H−tBu]⁺=260, HPLC: t_{Ret}=3.60, 4.22 min.

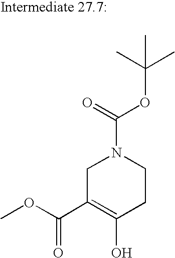

A mixture of methyl 4-oxo-3-piperidine carboxylate hydrochloride (10 g, 0.052 mol), Boc₂O (11.3 g, 0.052 mol), and Et₃N (7.24 ml, 0.052 mol) in THF (60 mL) is stirred at RT for 1 h. After adding NaHCO₃ aq., the reaction mixture is extracted with EtOAc. The combined organic phases are washed with brine and dried (Na₂SO₄) and concentrated under reduced pressure. The resulting residue is purified by silica gel flash chromatography to give Intermediate 27.7 as a colorless oil; ES-MS: [M+H−tBu]⁺=202, HPLC: t_{Ret}=3.22, 3.97 min.

The following Examples enlisted in Table 2 (as racemates or preferably in essentially pure form) are synthesized analogously to the preparation of the Example 27 or as hereinbefore described. As far as not being commercially available or available by synthesis analogous to methods or as described hereinbefore, the synthesis methods of intermediates for the preparation of compounds of the Examples 28-37 are described below Table 2. The asterisk (*) indicates the end of the bond at which the respective moiety is bound to the rest of the molecule falling under the following formula:

TABLE 2

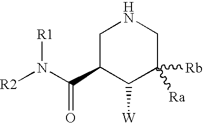

| Ex. | | | | | | Analytical |
|---|---|---|---|---|---|---|
| No. | R1— | R2— | Ra— | Rb— | W— | data |
| 28 | 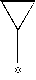 | 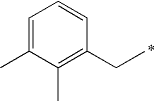 | 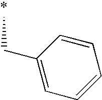 | 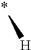 | 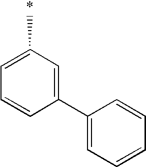 | MS: [M + 1]⁺ = 529 HPLC t_{Ret} = 4.35 RF = 0.42 (CH₂Cl₂/MeOH = 20:1) |
| 29 | 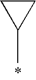 | 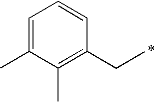 | 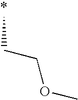 | 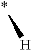 | 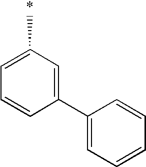 | MS: [M + 1]⁺ = 497 HPLC t_{Ret} = 3.90 RF = 0.69 (CH₂Cl₂/MeOH = 10:1) |
| 30 | 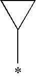 | 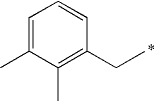 | 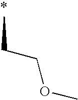 | 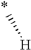 | 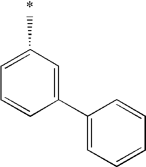 | MS: [M + 1]⁺ = 497 HPLC t_{Ret} = 3.90 RF = 0.63 (CH₂Cl₂/MeOH = 10:1) |
| 31 | 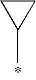 | 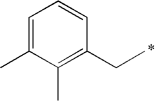 | 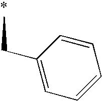 | 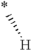 | 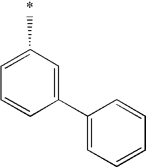 | MS: [M + 1]⁺ = 529 HPLC t_{Ret} = 4.35 RF = 0.29 (CH₂Cl₂/MeOH = 10:1) |
| 32 | 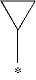 | 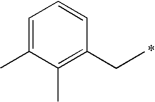 | 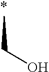 | 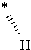 | 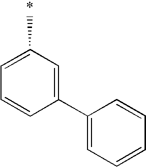 | MS: [M + 1]⁺ = 469 HPLC t_{Ret} = 3.34 |
| 33 | 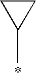 | 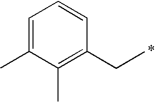 | 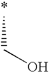 | 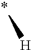 | 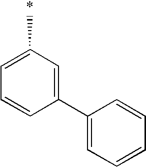 | MS: [M + 1]⁺ = 469 HPLC t_{Ret} = 3.45 |
| 34 | 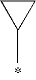 | 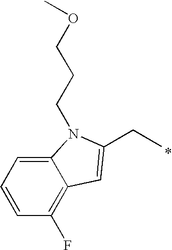 | 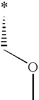 | 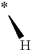 | 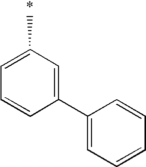 | MS: [M + 1]⁺ = 584 HPLC t_{Ret} = 3.65 |
| 35 | 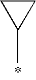 | 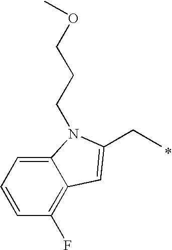 | 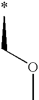 | 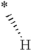 | 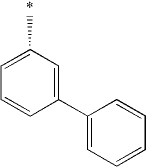 | MS: [M + 1]⁺ = 584 HPLC t_{Ret} = 3.73 |
| 36 | 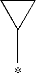 | 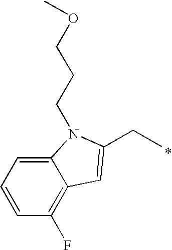 | 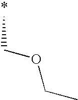 | 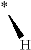 | 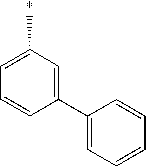 | MS: [M + 1]⁺ = 598 HPLC t_{Ret} = 3.72 |
| 37 | 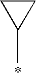 | 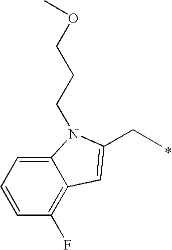 | 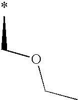 | 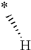 | 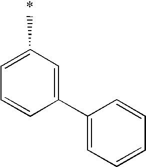 | MS: [M + 1]⁺ = 598 HPLC t_{Ret} = 3.70 |

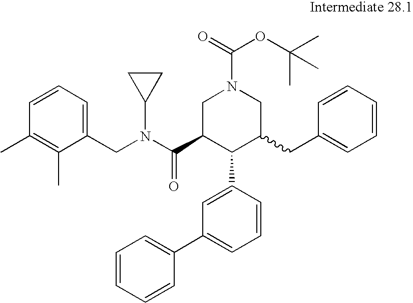

Intermediate 28.1 is synthesized by condensation of Intermediate 28.2 (44.1 mg, 0.094 mmol) and cyclopropyl-(2,3-dimethyl-benzyl)-amine (21.4 mg, 0.094 mmol) analogously to the preparation of Intermediate 27.1. White amorphous material; a white solid; ES-MS: M+H=629; HPLC: t_{Ret}=6.09 min.

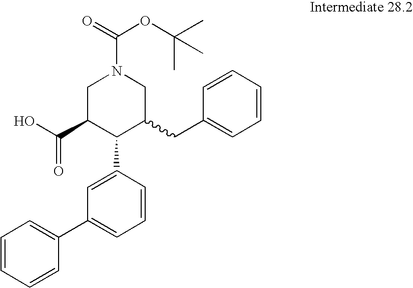

Intermediate 28.2 is synthesized by isomerization and hydrolysis of Intermediate 28.3 (894.9 mg, 1.84 mmol) analogously to the preparation of Intermediate 27.2. White amorphous material; a white solid; ES-MS: M+H−tBu=416; HPLC: t_{Ret}=5.20 min.

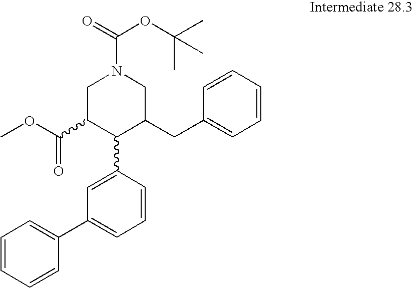

Intermediate 28.3 is synthesized by reduction of Intermediate 28.4 (1.46 g, 3.02 mmol) analogously to the preparation of Intermediate 27.3. White amorphous material; a white solid; ES-MS: M+H−tBu=430; HPLC: t_{Ret}=5.74 min.

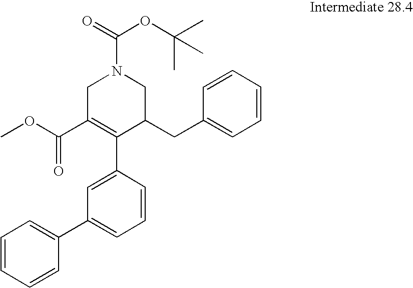

Intermediate 28.4 is synthesized by coupling of Intermediate 28.5 (1.56 g, 3.2 mmol) and 3-biphenyl boronic acid (773 mg, 3.9 mmol) analogously to the preparation of Intermediate 27.4. White amorphous material; a white solid; ES-MS: M+H−tBu=428; HPLC: _{A}t_{Ref}=5.80 min.

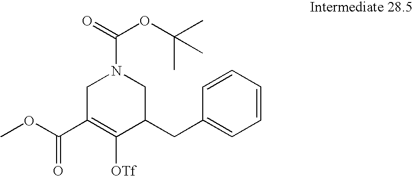

Intermediate 28.5 is synthesized by reaction of Intermediate 28.6 (1.5 g, 4.3 mmol) analogously to the preparation of Intermediate 27.5. White amorphous material; a white solid; ES-MS: M+H−tBu=424; HPLC: t_{Ret}=5.32 min.

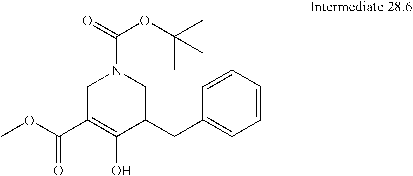

Intermediate 28.6 is synthesized by alkylation of Intermediate 28.7 (2.13 g, 7.8 mmol) and benzyl bromide (1.1 ml, 9.36 mmol) analogously to the preparation of Intermediate 27.6. White amorphous material; a white solid; ES-MS: M+H−tBu 292; HPLC: t_{Ret}=4.43, 5.11 min.

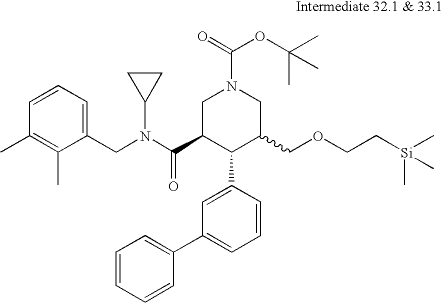

Intermediate 32.1 & 33.1 is synthesized by condensation of Intermediate 32.2 & 33.2 (174.5 mg, 0.34 mmol) and cyclopropyl-(2,3-dimethyl-benzyl)-amine (86.6 mg, 0.409 mmol) analogously to the preparation of Intermediate 27.1. Two diastereomers were separated by silica gel chromatography. White amorphous material; a white solid; ES-MS: M+H=669; HPLC: t_{Ret}=6.30 min.

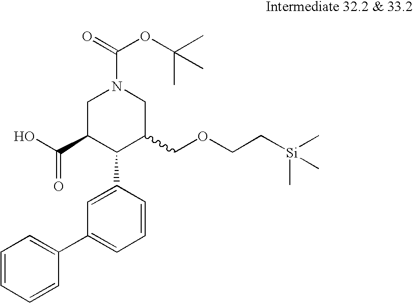

Intermediate 32.2 & 33.2 is synthesized by isomerization and hydrolysis of Intermediate 32.3 & 33.3 (230 mg, 0.437 mmol) analogously to the preparation of Intermediate 27.2. White amorphous material; a white solid; ES-MS: M+H=521; HPLC: t_{Ret}=5.55, 5.65 min.

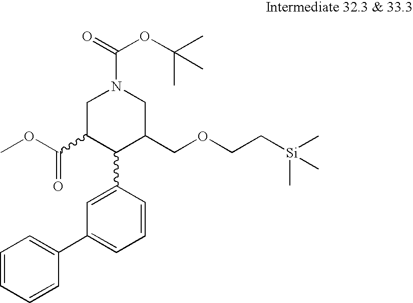

Intermediate 32.3 & 33.3 is synthesized by reduction of Intermediate 32.4 & 33.4 (222.5 mg, 0.42 mmol) analogously to the preparation of Intermediate 27.3. White amorphous material; a white solid; ES-MS: M+H=526; HPLC: t_{Ret}=6.22 min.

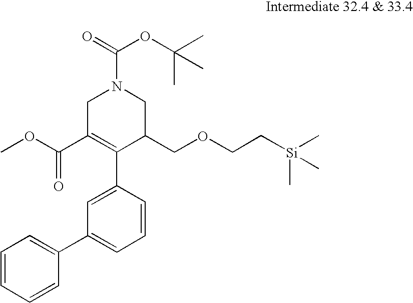

Intermediate 32.4 & 33.4 is synthesized by coupling of Intermediate 32.5 & 33.5 (291.5 mg, 0.561 mmol) and 3-biphenyl boronic acid (133.3 mg, 0.673 mmol) analogously to the preparation of Intermediate 27.4. White amorphous material; a white solid; ES-MS: M+H=524; HPLC: t_{Ret}=5.62min.

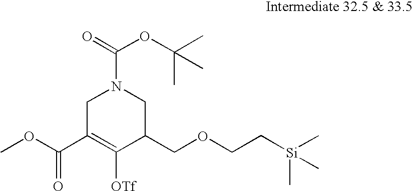

Intermediate 32.5 & 33.5 is synthesized by reaction of Intermediate 32.6 & 33.6 (438.6 mg, 1.13 mmol) analogously to the preparation of Intermediate 27.5. White amorphous material; a white solid; Rf (0.32, hexane/EtOAc=4/1) ; HPLC: t_{Ret}=5.25min.

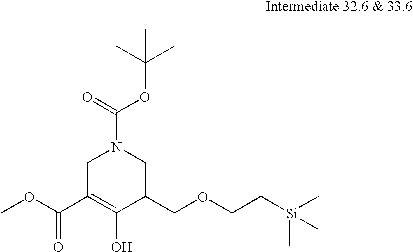

Intermediate 32.6 & 33.6 is synthesized by alkylation of Intermediate 27.7 (1.822 g, 6.6 mmol) and 2-(trimethylsilyl)ethoxymethyl choride (1.17 ml, 6.6 mmol) analogously to the preparation of Intermediate 27.6. White amorphous material; a white solid; Rf (0.57, hexane/EtOAc =4/1) HPLC: t_{Ret}=5.20, 5.82 min.

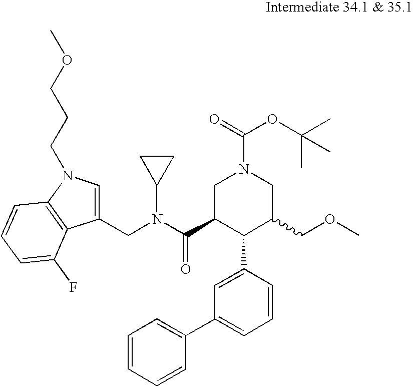

Intermediate 34.1 & 35.1 is synthesized by condensation of Intermediate 34.2 & 35.2 (315.3 mg, 0.74 mmol) and cyclopropyl-[4-fluoro-1-(3-methoxypropyl)-1H-indol-3-ylmethyl]amine (237.6 mg, 0.860 mmol) analogously to the preparation of Intermediate 27.1. White amorphous material; a white solid; ES-MS: M+H=684; HPLC: t_{Ret}=5.62 min.

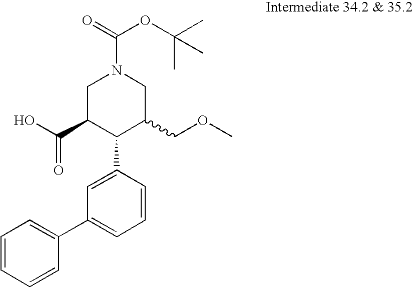

Intermediate 34.2 & 35.2 is synthesized by isomerization and hydrolysis of Intermediate 34.3 & 35.3 (2.04 g, 4.6 mmol) analogously to the preparation of Intermediate 27.2. White amorphous material; a white solid; ES-MS: M+H−tBu=370; HPLC: t_{Ret}=4.30 min.

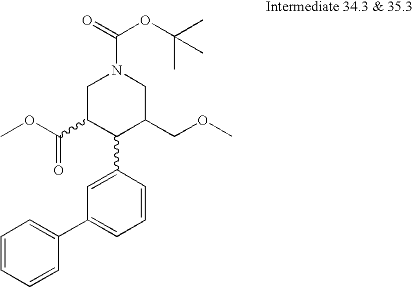

Intermediate 34.3 & 35.3 is synthesized by reduction of Intermediate 34.4 & 35.4 (1.147 g, 0.42 mmol) analogously to the preparation of Intermediate 27.3. White amorphous material; a white solid; ES-MS: M+H−tBu=384; HPLC: t_{Ret}=4.93, 5.02 min.

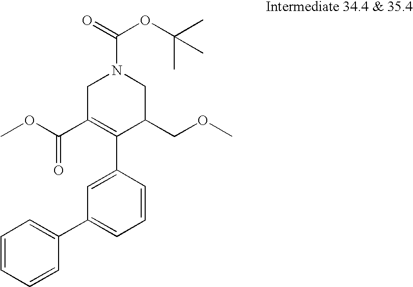

Intermediate 34.4 & 35.4 is synthesized by coupling of Intermediate 34.5 & 35.5 (2.09 g, 4.8 mmol) and 3-biphenyl boronic acid (1.43 g, 7.23 mmol) analogously to the preparation of Intermediate 27.4. White amorphous material; a white solid; ES-MS: M+H−Boc=338; HPLC: t_{Ret}=4.60 min.

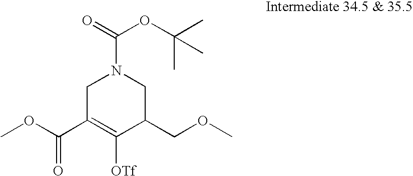

Intermediate 34.5 & 35.5 is synthesized by reaction of intermediate 34.6 & 35.6 (1.02 g, 3.4 mmol) analogously to the preparation of Intermediate 27.5. White amorphous material; a white solid; ES-MS: M+H−Boc=334; HPLC: t_{Ret}=4.14 min.

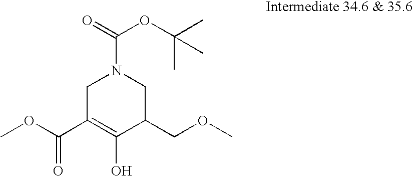

Intermediate 34.6 & 35.6 is synthesized by alkylation of Intermediate 27.7 (2.278 g, 8.3 mmol) and methoxymethyl choride (0.62 ml, 8.3 mmol) analogously to the preparation of Intermediate 27.6. White amorphous material; a white solid; ES-MS: M+H−Boc=202; HPLC: t_{Ret}=3.53,4.12 min.

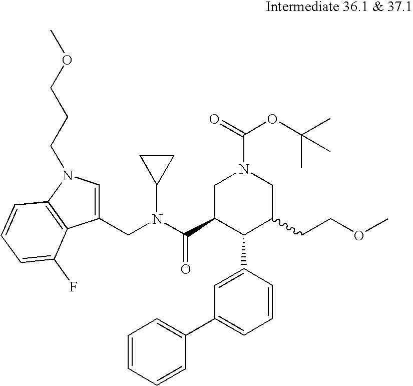

Intermediate 36.1 & 37.1 is synthesized by condensation of Intermediate 27.2 (131.8 mg, 0.3 mmol) and cyclopropyl-[4-fluoro-1-(3-methoxypropyl)-1H-indol-3-ylmethyl]amine (91.1 mg, 0.33 mmol) analogously to the preparation of Intermediate 27.1. White amorphous; ES-MS: M+H=698; HPLC: t_{Ret}=5.80 min.

### Example 38

### Soft Capsules

5000 soft gelatin capsules, each comprising as active ingredient 0.05 g of any one of the compounds of formula I mentioned in any one of the preceding Examples, are prepared as follows:

Composition

| | |
|---|---|
| Active ingredient | 250 g |
| Lauroglycol | 2 liters |

Preparation process: The pulverized active ingredient is suspended in Lauroglykol® (propylene glycol laurate, Gattefossé S. A., Saint Priest, France) and ground in a wet pulverizer to produce a particle size of about 1 to 3 μm. 0.419 g portions of the mixture are then introduced into soft gelatin capsules using a capsule-filling machine.

### Example 74

### Tablets Comprising Compounds of the formula I

Tablets, comprising, as active ingredient, 100 mg of any one of the compounds of formula I in any one of the preceding Examples 1 to 73 are prepared with the following composition, following standard procedures:

Composition

| | | |
|---|---|---|
| Active Ingredient | 100 | mg |
| crystalline lactose | 240 | mg |
| Avicel | 80 | mg |
| PVPPXL | 20 | mg |
| Aerosil | 2 | mg |
| magnesium stearate | 5 | mg |
| | 447 | mg |

Manufacture: The active ingredient is mixed with the carrier materials and compressed by means of a tabletting machine (Korsch EKO, stamp diameter 10 mm).

Avicel® is microcrystalline cellulose (FMC, Philadelphia, USA). PVPPXL is polyvinylpolypyrrolidone, cross-linked (BASF, Germany). Aerosil® is silicon dioxide (Degussa, Germany).

We claim:

1. A compound of the formula I 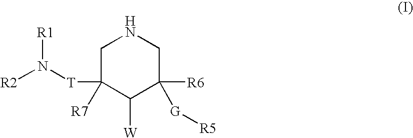
wherein
R1 is hydrogen, unsubstituted or substituted alkyl, unsubstituted or substituted alkenyl, unsubstituted or substituted alkynyl, unsubstituted or substituted aryl, unsubstituted or substituted heterocyclyl or unsubstituted or substituted cycloalkyl;
R2 is unsubstituted or substituted alkyl, unsubstituted or substituted alkenyl, unsubstituted or substituted alkynyl, unsubstituted or substituted aryl, unsubstituted or substituted heterocyclyl, unsubstituted or substituted cycloalkyl, or acyl;
W is a moiety selected from those of the formulae IA, IB and IC, 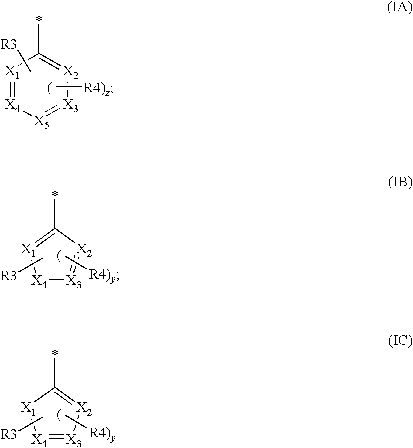
wherein the asterisk (*) denotes the position where the moiety W is bound to the 4-carbon in the piperidine ring in formula I, and wherein
X₁, X₂, X₃, X₄ and X₅ are independently selected from carbon and nitrogen, where X₄ in formula IB and X₁ in formula IC may have one of these meanings or alternatively be selected from S and O, where carbon and nitrogen ring atoms can carry the required number of hydrogen or substituents R₃ or if present within the limitations given below R₄ to complete the number of bonds emerging from a ring carbon to four, from a ring nitrogen to three; with the proviso that in formula IA at least 2 of X₁ to X₅ are carbon and in formulae IB and IC at least one of X₁ to X₄ is carbon;
y is 0, 1, 2 or 3;
z is 0, 1, 2, 3 or 4
R3 which can only be bound to any one of X₁, X₂, X₃ and X₄ is unsubstituted or substituted C₁-C₇-alkyl, unsubstituted or substituted C₂-C₇-alkenyl, unsubstituted or substituted C₂-C₇-alkynyl, unsubstituted or substituted aryl, unsubstituted or substituted heterocyclyl, unsubstituted or substituted cycloalkyl, halo, hydroxy, etherified or esterified hydroxy, unsubstituted or substituted mercapto, unsubstituted or substituted sulfinyl, unsubstituted or substituted sulfonyl, amino, mono- or di-substituted amino, carboxy, esterified or amidated carboxy, unsubstituted or substituted sulfamoyl, nitro or cyano, with the proviso that if R3 is hydrogen then y and z are 0;
R4 is—if y or z is 2 or more, independently—selected from a group of substituents consisting of unsubstituted or substituted C₁-C₇-alkyl, unsubstituted or substituted C₂-C₇-alkenyl, unsubstituted or substituted C₂-C₇-alkynyl, halo, hydroxy, etherified or esterified hydroxy, unsubstituted or substituted mercapto, unsubstituted or substituted sulfinyl, unsubstituted or substituted sulfonyl, amino, mono- or di-substituted amino, carboxy, esterified or amidated carboxy, unsubstituted or substituted sulfamoyl, nitro and cyano;
G is methylene, oxy, thio, imino or substituted imino —NR8- wherein R8 is unsubstituted or substituted alkyl; and
R5 is hydrogen, unsubstituted or substituted alkyl, unsubstituted or substituted aryl or acyl or, if G is methylene, can have one of these meanings or alternatively be unsubstituted or substituted alkyloxy;
or G-R5 is halo;
R6 is hydrogen, C₁-C₇-alkyl or halo;
or G-R5 and R6 together are oxo and/or G-R5 is hydroxy and R6 is hydroxy;
R7 is hydrogen, hydroxy, halo, C₁-C₇-alkyl, halo-C₁-C₇-alkyl, cycloalkyl, halo-substituted cycloalkyl, C₁-C₇-alkoxy, halo-C₁-C₇-alkoxy or cyano; and
is carbonyl;
or a salt thereof.

2. A compound of the formula I according to claim 1, wherein
R1 is hydrogen, unsubstituted or substituted alkyl, unsubstituted or substituted alkenyl, unsubstituted or substituted alkynyl, unsubstituted or substituted aryl, unsubstituted or substituted heterocyclyl or unsubstituted or substituted cycloalkyl;
R2 is unsubstituted or substituted alkyl, unsubstituted or substituted alkenyl, unsubstituted or substituted alkynyl, unsubstituted or substituted aryl, unsubstituted or substituted heterocyclyl, unsubstituted or substituted cycloalkyl, or acyl;
W is a moiety selected from those of the formulae IA, IB and IC as shown in claim 1, wherein the asterisk (*) denotes the position where the moiety W is bound to the 4-carbon in the piperidine ring in formula I, and wherein X₁, X₂, X₃, X₄ and X₅ are independently selected from carbon and nitrogen, where X₄ in formula IB and X₁ in formula IC may have one of these meanings or alternatively be selected from S and O, where carbon and nitrogen ring atoms can carry the required number of hydrogen or substituents R₃ or R₄ to complete the number of bonds emerging from a ring carbon to four, from a ring nitrogen to three; with the proviso that in formula IA at least 2, preferably at least 3 of X₁ to X₅ are carbon and in formulae IB and IC at least one of X₁ to X₄ is carbon, preferably at least two of X₁ to X₄ are carbon;
y is 0, 1, 2 or 3;
z is 0, 1, 2, 3 or 4
R3 which can only be bound to any one of X₁, X₂, X₃ and X₄ instead of a hydrogen and replacing it is unsubstituted or substituted C₁-C₇-alkyl, unsubstituted or substituted C₂-C₇-alkenyl, unsubstituted or substituted C₂-C₇-alkynyl, unsubstituted or substituted aryl, unsubstituted or substituted heterocyclyl, unsubstituted or substituted cycloalkyl, halo, hydroxy, etherified or esterified hydroxy, unsubstituted or substituted mercapto, unsubstituted or substituted sulfinyl, unsubstituted or substituted sulfonyl, amino, mono- or di-substituted amino, carboxy, esterified or amidated carboxy, unsubstituted or substituted sulfamoyl, nitro or cyano, with the proviso that if R3 is hydrogen then y and z are 0;
R4 which is preferably bound to a ring atom other than that to which R₃ is bound is—if y or z is 2 or more, independently—selected from a group of substituents consisting of unsubstituted or substituted C₁-C₇-alkyl, unsubstituted or substituted C₂-C₇-alkenyl, unsubstituted or substituted C₂-C₇-alkynyl, halo, hydroxy, etherified or esterified hydroxy, unsubstituted or substituted mercapto, unsubstituted or substituted sulfinyl, unsubstituted or substituted sulfonyl, amino, mono- or di-substituted amino, carboxy, esterified or amidated carboxy, unsubstituted or substituted sulfamoyl, nitro and cyano;
G is methylene, oxy, thio, imino or substituted imino —NR8- wherein R8 is unsubstituted or substituted alkyl; and
R5 is hydrogen, unsubstituted or substituted alkyl, unsubstituted or substituted aryl or acyl or, if G is methylene, can have one of these meanings or alternatively be unsubstituted or substituted alkyloxy;
or G-R5 is halo;
R6 is hydrogen, C₁-C₇-alkyl or halo;
or G-R5 and R6 together are oxo and/or G-R5 is hydroxy and R6 is hydroxy;
R7 is hydrogen, hydroxy, halo, C₁-C₇-alkyl, halo-C₁-C₋₇alkyl, cycloalkyl, halo-substituted cycloalkyl, C₁-G₇-alkoxy, halo-C₁-C₇-alkoxy or cyano; and
T is carbonyl;
or a pharmaceutically acceptable salt thereof.

3. A compound of the formula I according to claim 1, wherein
R1 is hydrogen, C₃-C₈-cycloalkyl, C₁-C₇-alkyl or halo-lower alkyl;
R2 is phenyl-C₁-C₇-alkyl wherein phenyl is unsubstituted or substituted by one or more, preferably up to three, moieties independently selected from the group consisting of C₁-C₇-alkyl, halo, C₁-C₇-alkyloxy-C₁-C₇-alkyloxy and C₁-C₇-alkyloxy; indolyl, or with slightly less preference benzoxazinonyl, indolyl- or benzoxazinonyl-C₁-C₇-alkyl, wherein where mentioned hereinbefore indolyl and benzoxazinyl is unsubstituted or substituted by one or more, preferably up to three, substituents independently selected from halo, C₁-C₇-alkyloxy and Cₗ-C₇-alkoxy-C₁-C₇-alkyl;
W is a moiety of the formula IA wherein one of X₁ and X₂ is nitrogen or CH, while the other and X₃, X₄ and X₅ are CH; or a moiety of the formula IC shown in claim 1, wherein X, is CH₂, NH, S or O and one of X₂, X₃ and X₄ is N, while the others are CH, with the proviso that at least one ring nitrogen N or in the case or X₁ NH is present;
z and y are 0;
R3 is phenyl or phenyl-C₁-C₇-alkoxy, where in both cases phenyl is unsubstituted or substituted by one or more, preferably up to three, moieties independently selected from hydroxy and C₁-C₇-alkyloxy; with the proviso that R3 in the case where W is a moiety of the formula IA is bound either to X₃ or to X₄ or to X₁ or to X₂, in the case where W is a moiety of the formula IA is bound either to X₃ or X₄, or to X₂; or (with slightly less preference) is pyridyl, e.g. 2-pyridyl;
G is methylene, oxy, thio, imino or substituted imino —NR8- wherein R8 is C₁-C₇-alkyl or unsubstituted or halo-substituted phenyl-C₁-C₇-alkyl;
R5 is hydrogen, C₁-G₇-alkyl that is unsubstituted or substituted by one or more, preferably up to three, moieties independently selected from phenyl, C₁-C₇-alkoxy and hydroxy; or, if G is methylene, can have one of the meanings just mentioned or alternatively be selected from phenyl, C₁-C₇alkoxy-C₁-C₇-alkyl, C₁-C₇-alkoxy-C₁-C₇-alkoxy-C₁-C₇-alkyl, C₁-C₇-alkoxy and C₁-C₇alkoxy-C₁-C₇-alkoxy; or, if G is oxy, thio, imino or —NR8-, can be selected from hydrogen, C₁-C₇-alkyl that is unsubstituted or substituted by one or more, preferably up to three, moieties independently selected from phenyl, C₁-C₇-alkoxy and hydroxy, C₁-C₇-alkanoyl, such as acetyl, 3,3-dimethyl-butyryl, 2,2-dimethyl-propionyl or 3,3-dimethyl-butyryl, unsubstituted or mono-, di- or tri-(halo and/or C₁-C₇-alkyl)-substituted benzoyl or naphthoyl, such as 4-methyl-benzoyl, C₃-C₈-cycloalkylcarbonyl, such as cyclobutylcarbonyl, unsubstituted or phenyl-substituted pyrrolidinylcarbonyl, especially phenyl-pyrrolidinocarbonyl, C₁-C₇-alkylsulfonyl, such as methylsulfonyl (=methanesulfonyl), (phenyl- or naphthyl)-C₁-C₇-alkylsulfonyl, such as phenylmethanesulfonyl, or (unsubstituted, or [C₁-C₇-alkyl-, phenyl-, halo-lower alkyl-, halo, oxo-C₁-C₇-alkyl-C₁-C₇-alkyloxy-, phenyl-C₁-C₇-alkoxy-, halo-C₁-C₇-alkyloxy-, phenoxy-, C₁-C₇-alkanoylamino-, cyano-, C₁-C₇-alkanoyl- and/or C₁-C₇-alkylsulfonyl-]substituted) (phenyl-or naphthyl)-sulfonyl, such as phenylsulfonyl, naphthalene-1-sulfonyl, naphthalene-2-sulfonyl, toluene-4-sulfonyl, 4-isopropyl-benzenesulfonyl, biphenyl-4-sulfonyl, 2-trifluoromethyl-benzenesulfonyl, 4-chloro-benzenesulfonyl, 3-chloro-benzenesulfonyl, 2-chloro-benzenesulfonyl, 2,4-difluoro-benzenesulfonyl, 2,6-difluoro-benzenesulfonyl, 2,5-dichloro-benzenesulfonyl, 3,4-dichloro-benzenesulfonyl, 3,5-dichloro-benzenesulfonyl, 2,3-dichloro-benzenesulfonyl, 3-methoxy-benzenesulfonyl, 4-methoxy-benzenesulfonyl, 2,5-dimethoxy-benzenesulfonyl, 4-trifluoromethoxy-benzenesulfonyl, 2-benzyloxy-benzenesulfonyl, 3-trifluoromethyl-benzenesulfonyl, 4-phenoxy-benzenesulfonyl, 4-(2-oxo-propyl)-benzenesulfonyl, 4-acetylamino-benzenesulfonyl, 4-cyano-benzenesulfonyl, 2-cyano-benzenesulfonyl, 3-cyano-benzenesulfonyl, 3-acetyl-benzenesulfonyl or 4-methanesulfonyl-benzenesulfonyl, halo-thiophene-2-sulfonyl, such as 5-chloro-thiophene-2-sulfonyl, quinoline-sulfonyl, such as quinoline-8-sulfonyl, (C₁-C₇-alkanoylamino and/or C₁-C₇-alkyl)-substituted thiazol-sulfonyl, such as 2-acetylamino-4-methyl-thiazole-5-sulfonyl, (halo and/or C₁-C₇-alkyl)-substituted pyrazolesulfonyl, such as 5-chloro-1,3-dimethyl-1H-pyrazole-4-sulfonyl, pyridine-sulfonyl, such as pyridine-3-sulfonyl, or N-mono- or N,N-di-(C₁-C₇-alkyl, (unsubstituted or halo-substituted) phenyl or naphthyl, phenyl-C₁-C₇-alkyl, naphthyl-C₁-C₇-alkyl or C₃-C₈-cycloalkyl)-aminocarbonyl, such as N-tert-butyl-aminocarbonyl, (3-chloro-phenyl)-aminocarbonyl, N-benzyl-aminocarbonyl, N-cyclohexyl-aminocarbonyl, C₁-C₇-alkylaminocarbonyl or phenyl-C₁-C₇-alkylaminocarbonyl, and (C₁-C₇-alkyl, phenyl, naphthyl, phenyl-C₁-C₇-alkyl and/or napthyl-C₁-C₇-alkyl)-oxycarbonyl, e.g. C₁-C₇-alkoxycarbonyl, such as tert-butyloxycarbonyl or isobutyloxycarbonyl, or phenyl-C₁-C₇-alkyloxycarbonyl; especially from C₁-C₇-alkanoyl, C₁-C₇-alkylsulfonyl, phenylsulfonyl, C₁-C₇-alkoxycarbonyl. or G-R5 is halo, especially fluoro;
R6 is hydrogen, C₁-C₇-alkyl or halo, especially fluoro;
or G-R5 and R6 together are oxo as such and/or in hydrated form as two hydroxy groups;
R7 is hydrogen; and
T is carbonyl;
or a pharmaceutically acceptable salt thereof.

4. A compound of the formula I according to claim 1 with a relative configuration given in the following formula A: 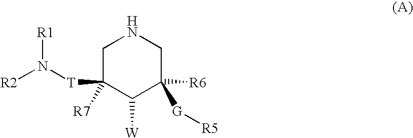
wherein G-R5 and R6 are not together oxo, or a pharmaceutically acceptable salt thereof.

5. A compound of the formula I according to claim 1 with a relative configuration given in the following formula B: 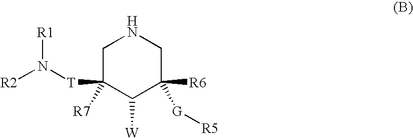
wherein G-R5 and R6 are not together oxo, or a pharmaceutically acceptable salt thereof.

6. A compound of the formula I according to claim 1, selected from the group of compounds with the formulae 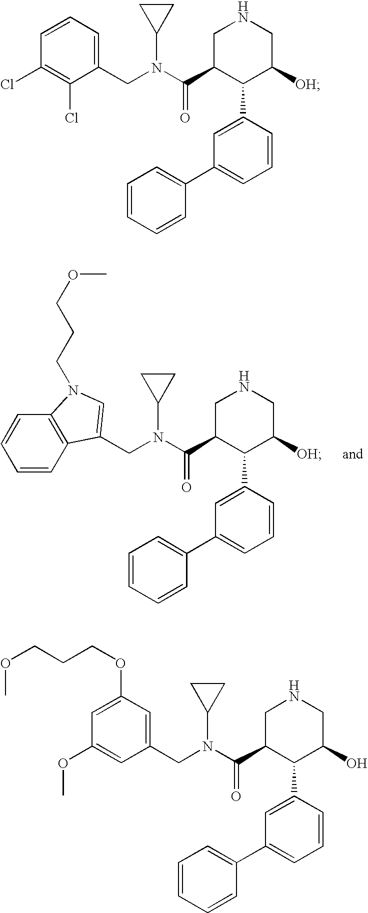
and/or the antipode thereof; or a pharmaceutically acceptable salt thereof.

7. A compound of the formula I according to claim 1, selected from the group of compounds of the formula 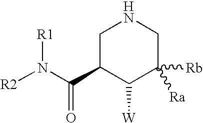
wherein R1, R2, W, Ra and Rb have the meanings given n the following table:

| Cpd. | | | | | |
|---|---|---|---|---|---|
| No. | R1— | R2— | Ra— | Rb— | W— |
| 4 | 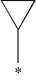 | 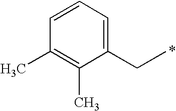 | 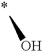 | 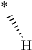 | 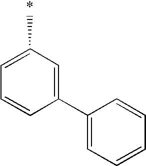 |
| 5 | 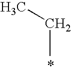 | 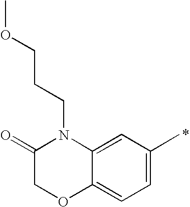 | 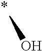 | 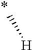 | 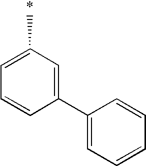 |
| 6 | 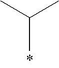 | 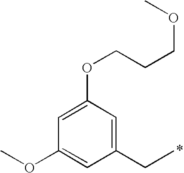 | 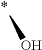 | 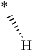 | 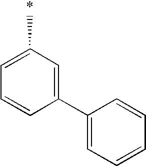 |
| 7 | 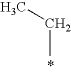 | 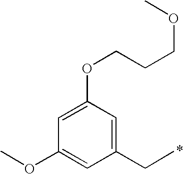 | 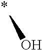 | 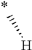 | 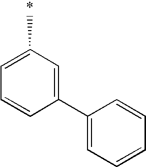 |
| 8 | 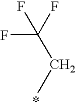 | 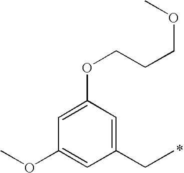 | 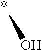 | 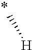 | 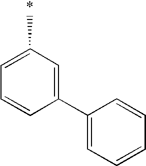 |
| 9 | 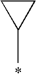 | 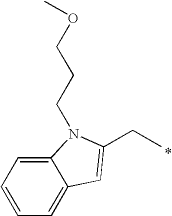 | 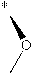 | 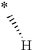 | 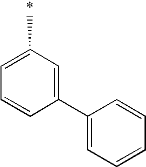 |
| 10 | 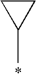 | 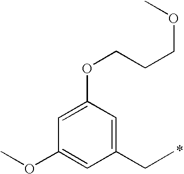 | 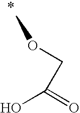 | 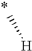 | 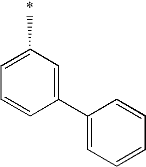 |
| 11 | 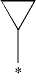 | 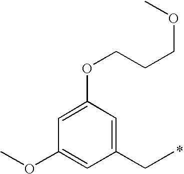 | 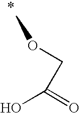 | 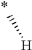 | 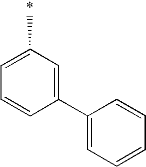 |
| 12 | 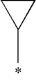 | 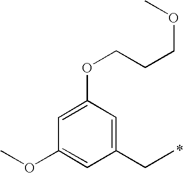 | 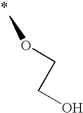 | 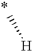 | 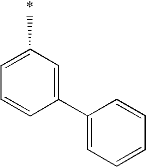 |
| 13 | 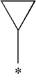 | 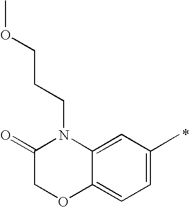 | 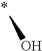 | 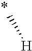 | 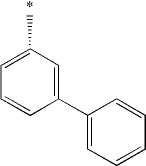 |
| 14 | 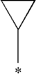 | 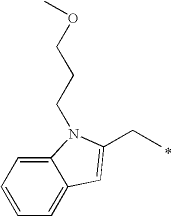 | 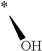 | 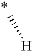 | 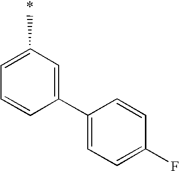 |
| 15 | 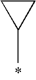 | 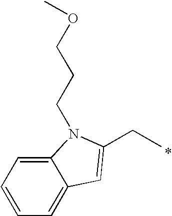 | 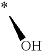 | 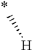 | 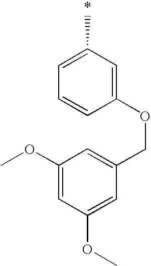 |
| 16 | 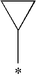 | 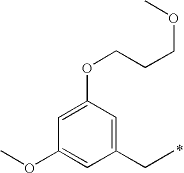 | 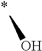 | 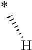 | 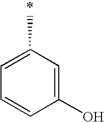 |
| 17 | 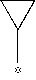 | 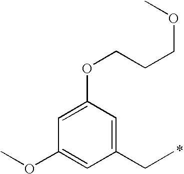 | 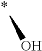 | 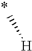 | 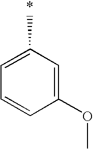 |
| 18 | 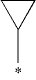 | 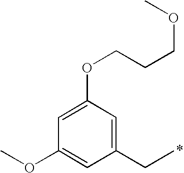 | 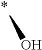 | 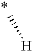 | 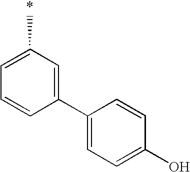 |

and/or the antipode thereof; or a pharmaceutically acceptable salt thereof.

8. A compound of the formula I according to claim 1, selected from the group of compounds with the formulae 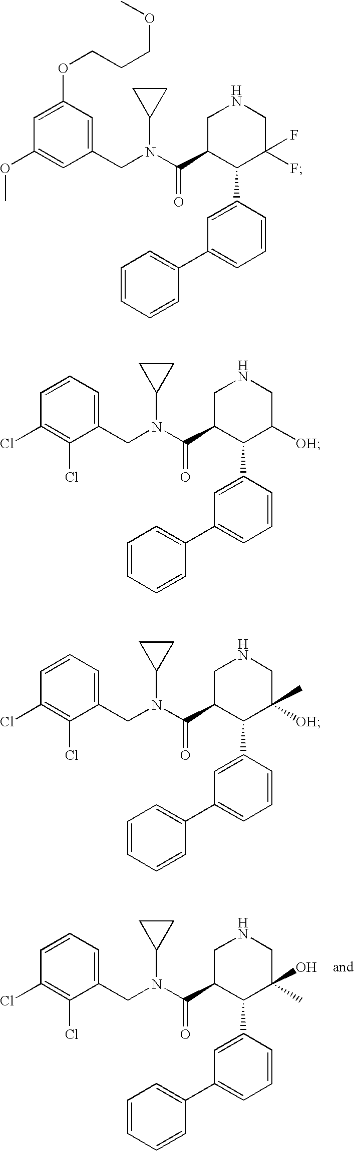 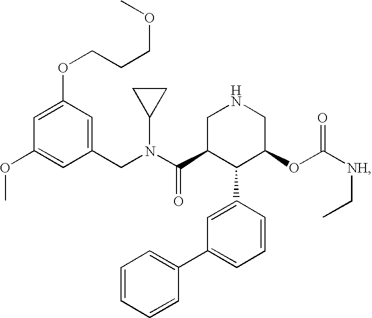
and/or the antipode thereof;
or a pharmaceutically acceptable salt thereof.

9. A compound of the formula I according to claim 1, selected from the group of compounds with the formulae 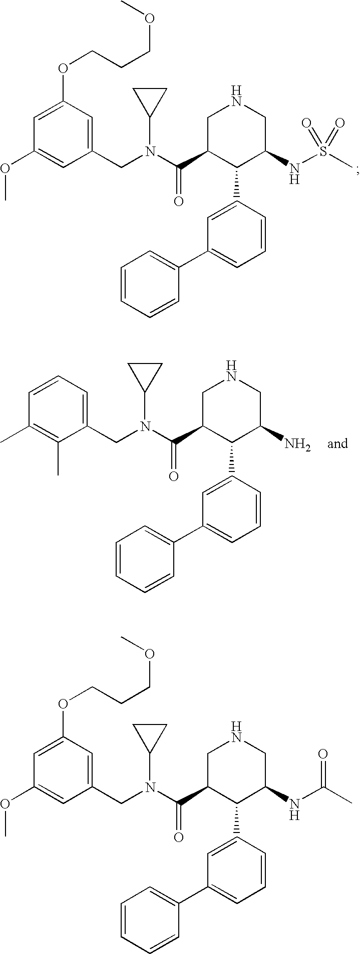
and/or the antipode thereof; or a pharmaceutically acceptable salt thereof.

10. A compound of the formula I according to claim 1 with the formula 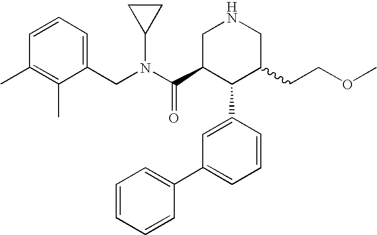
and/or the antipode thereof;or a pharmaceutically acceptable salt thereof.

11. A compound of the formula I according to claim 1, selected from the group of compounds of the formula 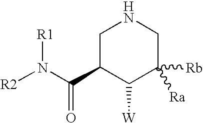
wherein R1, R2, W, Ra and Rb have he meanings given in the following table:

| Cpd. | | | | | |
|---|---|---|---|---|---|
| No. | R1— | R2— | Ra— | Rb— | W— |
| 28 | 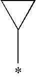 | 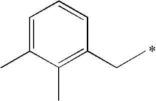 | 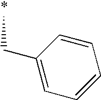 | 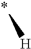 | 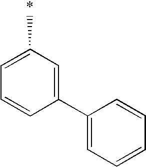 |
| 29 | 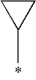 | 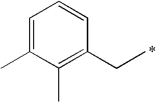 | 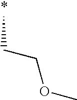 | 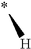 | 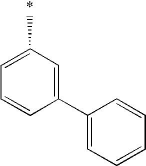 |
| 30 | 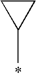 | 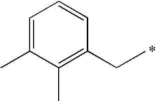 | 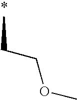 | 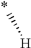 | 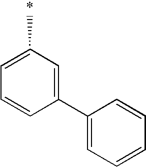 |
| 31 | 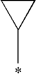 | 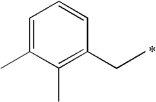 | 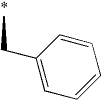 | 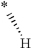 | 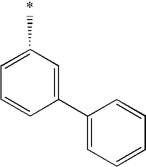 |
| 32 | 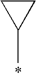 | 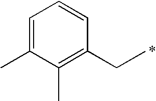 | 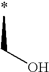 | 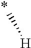 | 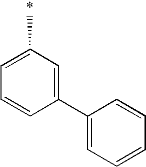 |
| 33 | 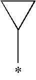 | 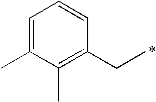 | 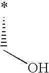 | 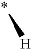 | 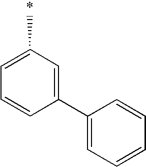 |
| 34 | 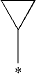 | 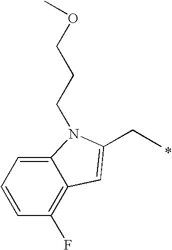 | 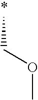 | 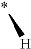 | 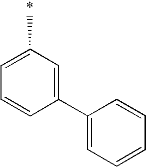 |
| 35 | 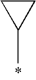 | 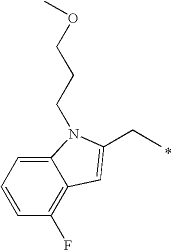 | 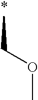 | 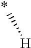 | 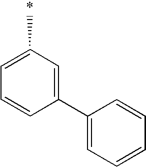 |
| 36 | 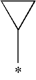 | 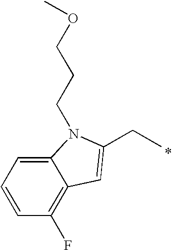 | 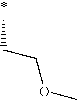 | 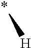 | 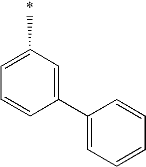 |
| 37 | 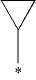 | 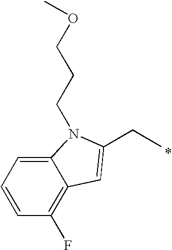 | 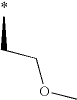 | 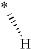 | 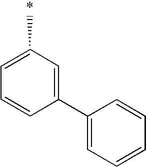 |

and/or the antipode thereof; or a pharmaceutically acceptable salt thereof.

12-15. (canceled)

16. A pharmaceutical formulation, comprising a compound of the formula I, or a pharmaceutically acceptable salt thereof, according to claim 1 and at least one pharmaceutically acceptable carrier material.

17. A method of treatment a disease that depends on activity of renin, comprising administering to a warm-blooded animal, especially a human, in need of such treatment a pharmaceutically effective amount of a compound of the formula I, or a pharmaceutically acceptable salt thereof, according to claim 1.

18. A process for the manufacture of a compound of the formula I according to claim 1, comprising
(a) reacting a carbonic acid compound of the formula II 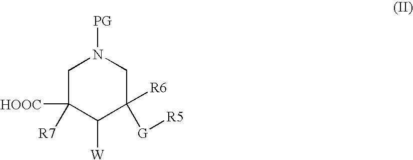
wherein W, G, R5, R6 and R7G-R5 are as defined for a compound of the formula I and PG is a protecting group, or an active derivative thereof, with an amine of the formula III, 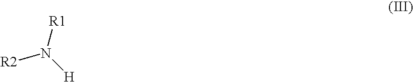
wherein R1 and R2 are as defined for a compound of the formula I, and removing protecting groups to give the corresponding compound of the formula I, or
(b) for the preparation of a compound of the formula I wherein R₃ is unsubstituted or substituted aryl or unsubstituted or substituted alkyloxy and W is a moiety of the formula IA given above, by reacting a compound of the formula IV, 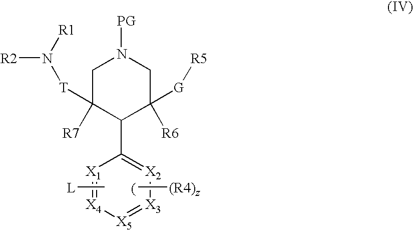
wherein R1, R2, T, G, R5, R6, R7, X₁, X₂, X₃, X₄, X₅, z and R4 are as defined for a compound of the formula I, PG is a protecting group and L is a leaving group or hydroxy, with a compound of the formula V,
R3-Q (V)
wherein R3 is as just defined and Q is —B(OH)₂ or a leaving group, and removing protecting groups to give the corresponding compound of the formula I,
and, if desired, subsequent to any one or more of the processes mentioned above converting an obtainable compound of the formula I or a protected form thereof into a different compound of the formula I, converting a salt of an obtainable compound of formula I into the free compound or a different salt, converting an obtainable free compound of formula I into a salt thereof, and/or separating an obtainable mixture of isomers of a compound of formula I into individual isomers;
where in any of the starting materials, in addition to specific protecting groups mentioned, further protecting groups may be present, and any protecting groups are removed at an appropriate stage in order to obtain a corresponding compound of the formula I, or a salt thereof.

19. A method of treating a patient suffering from a rennin dependent disease, the method comprising the steps of administering a compound of formula I of claim 1 or a pharmaceutically acceptable salt thereof to said patient.
